(19) United States
(12) Patent Application Publication
(10) Pub. No.: US 2024/0294624 A1
(43) Pub. Date: Sep. 5, 2024

(54) ANTI-ERYTHROPOIETIN ANTIBODY

(71) Applicant: ANDREMACON S.R.L., Milano (IT)

(72) Inventors: Giovanni Marfia, Milano (IT); Stefania Elena Navone, Milano (IT); Giuseppe Scalvini, Castelletto di Leno (IT); Rolando Campanella, Milano (IT); Giovanni Andrea Alotta, Altofonte (PA) (IT); Emanuela Barilla, Parma (IT)

(73) Assignee: ANDREMACON S.R.L., Milano (IT)

(21) Appl. No.: 18/573,034

(22) PCT Filed: Jul. 6, 2022
(86) PCT No.: PCT/EP2022/068805

§ 371 Date: Dec. 21, 2023

(30) Foreign Application Priority Data
Jul. 6, 2021 (WO) PCT/EP2021/068690

(51) Int. Cl. C07K 16/22; A61K 39/00; C07K 16/28; C12Q 1/6886; G01N 33/574; G01N 33/68

(52) U.S. Cl. CPC C07K 16/22; C07K 16/2866; C12Q 1/6886; G01N 33/57407; G01N 33/6863; A61K 2039/505; C07K 2317/732; C07K 2317/76; C07K 2317/92; C12Q 2600/156

(57) ABSTRACT

The present invention concerns the field of monoclonal antibodies, and describes an isolated anti-EPO antibody which binds human Erythropoietin (EPO) preventing its binding to specific receptors and inhibiting their signaling pathway. The invention further describes a polynucleotide encoding the anti-EPO antibody, a vector comprising the polynucleotide and a host cell comprising the vector. Furthermore, a method is described, for producing the antibody. The compounds of the invention, alone or in combination, are effective in the treatment of proliferative disorders such as cancers, where they cause the induction of apoptosis and overcome drug-resistance in cancer cells, cancer stem cells and in tumor endothelial cells, of autoimmune and non-autoimmune based chronic inflammatory diseases, in the treatment of patients undergoing organ or tissue transplant, in the treatment of haemophilic arthropathy, neurodegenerative diseases and neurological diseases in which neuro inflammation plays a role in pathogenesis, for example: multiple sclerosis, Parkinson's disease, Alzheimer's disease, frontotemporal dementia, dementia with Lewy bodies, autoimmune disease with neurologic involvement, Amyotrophic Lateral Sclerosis, Neuromuscular Diseases, ophthalmic pathologies such as neovascular age related (NVAMD), macular degeneration, retinal vein occlusion (RVO), metabolic syndromes, diabetes, and neuropathic pain disorders and the invention described compositions comprising them and medical uses of the composition. In a further aspect, the invention discloses the antibody, composition, or immunoconjugate for use as a medicament.

### FIELD OF THE INVENTION

The present invention concerns the field of monoclonal antibodies and describes an isolated anti-EPO antibody which binds human Erythropoietin (EPO) preventing its binding to specific receptors and inhibiting their signaling pathway. The invention further describes a polynucleotide encoding the anti-EPO antibody, a vector comprising the polynucleotide and a host cell comprising the vector.

Furthermore, a method is described, for producing the antibody.

The compounds of the invention, alone or in combination, are effective in the treatment of proliferative disorders such as cancers, where they cause the induction of apoptosis and overcome drug-resistance in cancer cells, cancer stem cells and in tumor endothelial cells, of autoimmune and non-autoimmune based chronic inflammatory diseases, in the treatment of patients undergoing organ or tissue transplant, in the treatment of haemophilic arthropathy, neurodegenerative diseases and neurological diseases in which neuro inflammation plays a role in pathogenesis, for example: multiple sclerosis, Parkinson's disease, Alzheimer's disease, frontotemporal dementia, dementia with Lewy bodies, autoimmune disease with neurologic involvement, Amyotrophic Lateral Sclerosis, Neuromuscular Diseases, ophthalmic pathologies such as neovascular age related (NVAMD), macular degeneration, retinal vein occlusion (RVO), metabolic syndromes, diabetes, and neuropathic pain disorders and the invention described compositions comprising them and medical uses of the composition. In a further aspect, the invention discloses the antibody, composition, or immunoconjugate for use as a medicament.

### STATE OF THE ART

Monoclonal antibodies represent the fastest growing market segment in the pharmaceutical industry. Despite a number of disadvantages, they are particularly appreciated among biotherapists for their unique characteristics, such as a high target specificity, favorable pharmacokinetics (high half-life), as well as fast development and a high rate of success when compared to small molecules.

In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Today, millions of people are suffering from cancer or had cancer. Currently available therapeutic options neglect the individuality of each patients' disease and only temporarily influence tumor progression with poor effect on overall survival. Neoplasms are a group of diseases characterized by the uncontrolled growth and invasiveness and spread of abnormal cells.

The involvement of cancer stem cells (CSCs) and tumor endothelial cells (TECs) in the formation and development of the neoplasm is now evident. In fact, experimental evidence has shown that CSCs hierarchically guide tumor growth, also through bidirectional communication with the vascular compartment. CSCs are responsible for the processes of initiation and maintenance of the tumor and also for its resistance to therapeutic treatments and, consequently, for the presence of relapses. As such, CSCs constitute an important therapeutic target but the mechanisms underlying their pathobiology are still poorly known, consequently making it difficult to identify molecules capable of affecting them.

There is an urgent and a strong need for new anti-cancer therapeutic approaches, counteracting stem cell biology, in particularly in aggressive solid tumors, such as glioblastoma, the most common and most aggressive malignant tumor of the central nervous system, anaplastic astrocytomas, glioblastoma, colon cancer, lung cancer, breast cancer, ovarian cancer, prostate cancer, bladder cancer, and in hematological neoplasms such as leukemia.

Inflammation is an innate nonspecific defense mechanism, which constitutes a protective response of the organism resulting in the harmful action of physical, chemical and biological agents, and whose ultimate goal is the elimination of the initial cause of cell or tissue damage or an autoimmune reaction. The normal inflammatory response is an acute process that is resolved after removal of the stimulus that caused it. In contrast, when the inflammatory response progresses, either due to repeated exposure to a stimulus, or when the causative agent is not suitably removed, the process becomes chronic. Depending on the tissue and on the phase of inflammation in which it is found, there is activation of different cell types. Inflammation can be triggered by autoimmune phenomena of recognition by the immune system by “self” antigens.

Neuroinflammation in particular is an inflammatory “cytokine-mediated” process that can be caused by systemic tissue damage or, more often, by direct damage to the central nervous system (CNS). Neuroinflammation differs from inflammation by the reduced presence of lymphatic vessels within the brain parenchyma; the lack of endogenous cells capable of presenting the antigen and the presence of the blood-brain barrier, which reduces the exchange of immune cells and inflammation mediators within the bloodstream. The persistence of the inflammatory processes in the CNS can cause serious damage to the neural complex and compromise its functional integrity.

Neuroinflammation may have different origins such as a biological origin, for example ischemia; bacterial infections; the deposit of biological material (as occur in neurodegenerative diseases such as: Alzheimer's and Parkinson's); intracellular and extracellular storage diseases that trigger neuroinflammation, a traumatic origin, such as brain trauma, and an autoimmune origin. All these conditions are able to activate the innate immune response in the CNS.

Microglial cells represent 5-10% of the total cell population in the brain. It is a population of hematopoietic derivation: during embryogenesis, in fact, a subpopulation of monocytes migrates in the nervous system and differentiates into resident macrophages.

The microglia is normally dormant in the CNS, the cell soma remains almost motionless while the branches move constantly to monitor their surroundings. The occurrence of physiological changes in the environment, such as increased serum proteins, glutamate toxicity, deposits of amyloid, Tau and phospho-Tau protein and amorphous substances, increase of purines (ATP, ADP) or the presence of lipopolysaccharide (the molecule present the membrane of Gram-negative bacteria) are all stimuli that are able to activate microglia by different receptors and signaling pathways. The microglial cells present in the perivascular areas also exert the function of antigen-presenting cells (APC) on myelin-specific T cells, which have infiltrated the CNS and that may begin the inflammatory processes. When the microglia is activated, it assumes its phagocytic capacity, in order to eliminate the residue of any dead cells or bacteria and viruses. The main role of activated microglia is to promote and support the inflammation state through the production of cytokines, reactive oxygen intermediates, proteinase, complement factors and chemokines. Such inflammatory mediators promote the infiltration of immune cells from the bloodstream, the recruitment of other microglial cells from the surrounding areas and the activation of astrocytes. When the inflammatory stimulus that triggered the activation fails, the microglia participate in the suppression processes of the inflammatory state with the production of immunomodulatory cytokines, such as IL-15, and anti-inflammatory, such as IL-10; subsequently returning to a state of inactivation, or undergoing apoptosis. The microglial activation and neuroinflammatory events that follow are directed to neuroprotection and the elimination of the cause of homeostasis failure. In reality, both in neurodegenerative diseases of a chronic nature and in traumatic events, such as ischemia, uncontrolled and persistent microglial activation may have neurotoxic effects and contribute to exacerbate neuronal damage. Much evidence has demonstrated that the modulation of microglia activation, and the inflammatory state in the brain in general, is able to improve the symptoms of many pathological conditions and to decrease the phenomenon of neurodegeneration. Based on these observations, microglial activation represents a potential pharmacological target for the treatment of neurodegenerative and inflammatory diseases. In addition, the balance between neuroprotective and neurotoxic action of microglia is determined by several factors, including the nature of the stimulus and the microglial interactions with the other cells of the immune system. Macrophages, are effector cells of the innate immune system that phagocytose bacteria and secrete both pro-inflammatory and antimicrobial mediators. A large body of evidence revealed that macrophages, with different phenotypes coexist in tumors. In particular, Tumor-associated macrophages (TAMs) account for the majority of tumor bulk and are well known as a key player in the tumor microenvironment, inducing proliferation, migration, invasion, and survival of cancer cells. Furthermore, TAMs account for 30-50% of GBM tumor bulk, so targeting TAMs may be a reasonable and promising adjunctive therapy for these difficult-to-control cancers. More recently, it has been reported that EPO could directly modulate the activation, differentiation and function of immune cells towards pathological tissues, by inducing the expression of cytokines enrolled in inflammation and response, such as Tumor Necrosis Factor (TNF), Interferon (IFN), Interleukines (IL), such as IL-6, IL1, and Tumor Growth Factor beta (TGFb). Indeed, EPO can directly affect the polarization of macrophages from the classical activated M1 phenotype, toward the M2 phenotype to exert anti-inflammatory function and promote tissue healing. Recently, some studies revealed that EPO plays an immunoregulatory effect by acting also on T cells.

Hemophilic arthropathy is considered to be an inflammatory-like illness. In the context of chronic inflammation, hemophilic arthropathy (linked to a deficit of factor VIII/IX) represents a specific framework characterized by synovial hyperplasia supported by increased angiogenesis tumor-like aberrant features. This framework involves an increased frequency of bleeding intra-articular until complete destruction of tissues resulting in ankylosis and complete loss of motor function. The replacement therapy currently available based on the use of concentrates of factor VIII/IX is not able to prevent the development of joint damage. Instead, therapies that interfere with angiogenesis, synovial proliferation and the intrinsic inflammation process that follows, can interrupt the vicious circle of synovitis-bleeding-inflammation.

Human erythropoietin (Epo) is a 30.4 kDa glycoprotein produced and secreted mainly by the kidneys. Epo is normally present in the bloodstream where it represents the main erythropoietic hormone. Epo is responsible for regulating the production of red blood cells, by stimulating the differentiation and proliferation of erythroid progenitors, as well as maintaining the erythroid series.

The synthesis of Epo is controlled by a very sensitive feedback system whose production and secretion depends on alterations in the oxygen supply. Indeed, EPO synthesis is based on the presence of the transcription factor Hypoxia Inducible Factor (HIF). At the same time, hypoxia also plays a key role in controlling tumor growth and angiogenesis and constitutes an effective tumor adaptation and survival mechanism. The genes involved in the hypoxia signaling pathway are overexpressed by the CSCs in the hypoxic vascular/perinecrotic niche, but not by the transitional tissue present at the resection margin, considered “disease free” in anatomopathological terms.

The use of EPO and its derivatives is well known in the treatment of anemia from renal failure, reduced erythropoiesis and in combination with myelosuppressive chemotherapy regimens in the treatment of malignancies.

In parallel, some meta-analyses have demonstrated that erythroid stimulating agents (ESAs), as EPO, significantly shorten overall survival of cancer patients. Although mechanisms underlying ESA-associated decreases of overall survivals remain uncharacterized, EPO might promote tumour progression and metastasis via complex processes, as stimulating angiogenesis, facilitating metastatic niche formation, and antagonizing therapeutic efficacies to other therapies. Body of evidence demonstrated increased proliferation of tumor cells in response to exogenous recombinant EPO (rEPO) in breast cancer cells, in cells derived from carcinoma of the kidney and in renal carcinoma cells. A rEPO-mediated induction of proliferation and stimulation of invasion was reported in human head and neck squamous cell carcinoma, and a correlation between disease progression and expression of EPO receptors, was demonstrated. These preclinical data suggest that the exploration of strategies to block EPO function to target tumor growth and angiogenesis may be warranted.

Indeed, our previous research shows that EPO works as a growth factor for glioblastoma cancer cells and that blocking the signaling pathway by a monoclonal antibody is able to inhibit the growth of both the cancer stem cells and tumor endothelial cells, to induce apoptosis, to decrease the endothelial cell functionality through inhibition of vascular structure formation and migration (WO/2015/189813).

Moreover, WO/2015/189813 describes the use of negative functional modulators of EPO in glioblastoma (GBM), lung and colon cancer and in neuroinflammatory diseases, where negative functional modulators of EPO are able to counteract the activation process of pathological microglia.

The Epo signaling is mediated by its binding to a surface receptor (EpoR), a transmembrane glycoprotein (PM: 66-78 kDa) belonging to the superfamily of cytokine receptors, mainly located on progenitors present in the bone marrow.

The expression of EpoR in non-hematopoietic cells, such as vascular endothelial cells, in the kidneys, myoblasts and intestines demonstrates that non-hematopoietic biological effects of Epo-EpoR signaling exist. In particular, recent studies have reported the expression of EpoR in tissue biopsies of breast cancer, malignant ovarian tumors, in melanoma and in renal cell carcinoma, suggesting a pivotal role for Epo-EpoR signaling in controlling cancer cell proliferation.

There are two forms of EpoR: a homodimeric, responsible for the erythropoietic effects, and a heterodimeric, composed of an EpoR chain and a β-common receptor chain (βcR, CD131, colony-stimulating factor 2 receptor-β, CSF2RB). This second receptor is responsible for the non-erythropoietic effects of Epo at heart, nervous system, intestine, uterus, kidney and pancreatic islets. The activation of the EpoR/CD131 heterodimer requires much higher concentrations of Epo than those necessary for the activation of the homodimeric EpoR. In particular, both induce the activation of PI3K and MAPK, the phosphorylation of STAT5 and the regulation of the binding activity of members of the NF-kB family.

The presence of a third receptor, called ephrin-type B receptor 4 (EphB4), has also been demonstrated. EphB4 predominantly interacts with Ephrin B2, but is also capable of acting as a functional Epo receptor. Studies conducted on ovarian cancer cells, which constitutively express both EphB4 and EpoR, have shown that both ephrin-B2 and Epo directly activate EphB4, causing increased proliferation and invasive migration mediated by Scr kinase, and STAT3. Experimental studies demonstrated a low binding affinity of Epo for EphB4 (KD of 880 nM), compared to a KD of 28 nM for EpoR. Furthermore, in a clinical study it was observed that the survival of patients with breast cancer was significantly reduced with high expression of EphB4, but not of EpoR at the level of the tumor cells and that treatment with Epo significantly decreased survival. This indicated that Epo supported tumor growth, particularly by activating the mechanisms initiated by EphB4.

A molecule primarily involved in cancer progression is sphingosine-1-phosphate (S1 P), a bioactive lysolipid, produced from sphingosine (Sph) through phosphorylation by kinases (SK1/2), which contributes to cancer progression by regulating tumor proliferation, invasion, and angiogenesis. S1 P exerts its effects in the extracellular milieu by binding five specific cell surface G protein-coupled receptors (S1 P1-5). Marfia and colleagues demonstrated that S1 P acts on GBM (Marfia G, et al. Autocrine/paracrine sphingosine-1-phosphate fuels proliferative and stemness qualities of glioblastoma stem cells. Glia. 2014 December; 62(12):1968-81. doi: 10.1002/glia.22718. Epub 2014 Jul. 5. PMID: 25042636.)] on two different levels: i) as a local mediator, enhancing CSC survival and proliferation, TEC migration and tube formation; and ii) as a systemic effector, travelling through blood circulation and interacting with many organs and related functional mechanisms. In this regard, WO/2015/189813 teaches that the treatment with anti-EPO antibody significantly inhibits the intracellular synthesis of S1 P through the downregulation of SK1, an anti-apoptotic enzyme whose high levels in cancer tissues correlate with short survival of GBM patients. Interestingly, anti-EPO antibody reduces the secretion of S1 P into the extracellular environment and increase the intracellular levels of ceramide, a sphingolipid recognized as pro-apoptotic mediator, antagonist of S1 P. Surprisingly, the co-administration of anti-EPO antibody with FTY720 (Gilenya®), a functional antagonist of S1 P currently FDA approved for the treatment of multiple sclerosis, has been proved to be more effective in terms of cancer cell apoptosis, suggesting a synergic anti-cancer activity. Finally, performing comparative genomic hybridization analysis by array-CGH on 10 glioblastoma tissues and matched primary cancer stem cells, we previously discovered that all primary cancer stem cells present a trisomy of the chromosomic region 7q11.2-36.3 containing EPO gene, reporting 3 copies of EPO gene.

The need and importance is increasingly felt for the identification of an effective therapeutic treatment which would allow to block the proliferation of cancer cells, but also cancer stem cells in an effective manner.

It is therefore object of the present invention the development of compounds which bind human EPO preventing its binding to specific receptors and inhibiting their signaling pathway.

### SUMMARY OF THE INVENTION

The problem underlying the present invention is that of making available compounds for the treatment of cancer, where said compounds induce apoptosis in cancer stem cells and in tumor endothelial cells in order to allow for the manufacture of medicaments destined for the therapy of related neoplastic pathologies. Said compounds were surprisingly seen to be active in the treatment of other pathologies, which will be discussed in the detailed description of the invention, in which erythropoietin is involved. This problem is resolved by the present finding by the use of negative functional modulators, namely monoclonal antibodies, capable of functionally interacting with the biosynthetic pathway of EPO and which bind human EPO preventing its binding to specific receptors and inhibiting their signaling pathway.

The present invention concerns in a first aspect an isolated anti-EPO antibody, also identified as C4 antibody, wherein said antibody comprises:
a. a variable domain of a light chain (VL) having the amino acid sequence of SEQ ID NO:6; and
b. a variable domain of a heavy chain (VH) having the amino acid sequence of SEQ ID NO:14.

Said anti-EPO antibody can be produced by hybridoma or by phage display techniques. The hybridoma which produces the C4 antibody according to the present invention, said antibody comprising a variable domain of a light chain (VL) having the amino acid sequence of SEQ ID NO:6; and a variable domain of a heavy chain (VH) having the amino acid sequence of SEQ ID NO:14, was deposited at the Leibniz-Institute DSMZ with the accession number DSM ACC 3370 on 09.09.2021, according to the requirements of the Budapest Treaty on the international recognition of the deposit of microorganisms for patent procedure.

In a second aspect, herein described is a polynucleotide encoding the anti-EPO antibody according to the present invention.

In a further aspect, the invention provides for a vector comprising the polynucleotide encoding the anti-EPO antibody, wherein the vector is optionally an expression vector. In a still further aspect, described herein is a host cell comprising the vector of the present invention.

In a fifth aspect, the invention provides for an immunoconjugate comprising the anti-EPO antibody of the present invention conjugated to an agent, wherein said agent is chosen from the group consisting of a drug or cytotoxic agent or co-administered in combination with a negative functional modulator of S1 P signaling, and/or anti-EPO receptors selected from the group comprising EPOR, EPHB4, CSFR2B, tissue protection factor (TPR, EPOR/CD131 heterodimer), and/or EPO mimetics.

A further aspect of the invention describes a method for producing the anti-EPO antibody of the invention or the immunoconjugate comprising said anti-EPO antibody, said method comprising (a) expressing the vector comprising the polynucleotide encoding the anti-EPO antibody in a suitable host cell, and (b) recovering the antibody or immunoconjugate.

In a still further aspect, herein described is a pharmaceutical composition comprising (i) the anti-EPO antibody of the present invention or (ii) the polynucleotide encoding said anti-EPO antibody, wherein the composition optionally further comprises a carrier. In another aspect, the invention provides for an antibody comprising (a) a variable domain of a light chain (VL) having the amino acid sequence of SEQ ID NO:6; and
(b) a variable domain of a heavy chain (VH) having the amino acid sequence of SEQ ID NO:14, the composition or the immunoconjugate as herein described, for use as a medicament.

In a further aspect, the invention provides for an antibody comprising (a) a variable domain of a light chain (VL) having the amino acid sequence of SEQ ID NO:6; and
(b) a variable domain of a heavy chain (VH) having the amino acid sequence of SEQ ID NO:14, the composition or the immunoconjugate as herein described, for use in the treatment of a tumor, cancer, or cell proliferative disorder, and/or for inhibiting angiogenesis or vascular permeability, autoimmune and non-autoimmune based chronic inflammatory diseases, in the treatment of patients undergoing organ or tissue transplant, in the treatment of haemophilic arthropathy, and neurodegenerative diseases and neurological diseases in which neuroinflammation plays a role in pathogenesis, such as multiple sclerosis, Parkinson's disease, Alzheimer's disease, frontotemporal dementia, dementia with Lewy bodies, autoimmune disease with neurologic involvement, Amyotrophic Lateral Sclerosis, Neuromuscular Diseases and ophthalmic pathologies, such as neovascular age related (NVAMD), macular degeneration, retinal vein occlusion (RVO), metabolic syndromes, diabetes, and neuropathic pain disorders

In a further aspect, the invention describes a method of treatment comprising the step of administering anti-EPO antibody of the present invention, said composition or said immunoconjugate to a subject in need thereof.

In a still further aspect, the present invention describes a diagnostic method for measuring the amount of EPO protein in a sample previously obtained from a human or animal subject, comprising the step of using the C4 antibody of the invention.

In a still further aspect, herein described is a pharmaceutical kit comprising the antibody of the invention and one or more compounds chosen from the group consisting of a negative functional modulator of S1 P signaling or of an anti-EPO receptor selected from the group comprising EPOR, EPHB4, CSFR2B, tissue protection factor (TPR, EPOR/CD131 heterodimer), and/or EPO mimetics, for simultaneous, separate or sequential administration.

In a still further aspect, the invention relates to a hybridoma which is deposited under deposit Accession No. DSM ACC 3370 by the International Deposit Authority DSMZ, Braunschweig, Germany.

In a further aspect the invention describes an anti-Erythropoietin (EPO) monoclonal antibody produced by the hybridoma deposited under deposit Accession No. DSM ACC 3370 by the International Deposit Authority DSMZ, Braunschweig, Germany.

The anti-Erythropoietin (EPO) monoclonal antibody produced by the hybridoma deposited under deposit Accession No. DSM ACC 3370, comprises a variable domain of a light chain (VL) having the amino acid sequence of SEQ ID NO:6; and b. a variable domain of a heavy chain (VH) having the amino acid sequence of SEQ ID NO:14.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-30, wherein:

FIG. 1. FIG. 1A. Neutralizing test performed on cell-based assay. Nineteen anti-EPO hybridoma culture supernatants were compared for the ability to inhibit EPO binding to one of EPO receptors, by evaluating inhibition of cancer cell viability. From all anti-EPO hybridomas assessed, anti-EPO (C4) has been demonstrated to show the higher neutralizing activity.

FIG. 1B. VH and VL PCR amplification results VL1-VL2: VL (molecular weight around 500 bp) were amplified using 2 different sets of primers to increase chance of success. VH1-VH2: VH (molecular weight around 500 bp) were amplified using 2 different sets of primers to increase chance of success MW: molecular weight standard (DL2000).

FIG. 1C. PCR validation after cloning: Clones 1-4, 6-12 of 6-E2-H5-2-B8-VL and clones 3, 8-12 of 6-E2-H5-2-B8-VH were at correct size (around 500 bp) so were sequenced. Clones 1-6, 8-12 of 6-E2-D5-2-C4-VL and clones 1-3, 5, 8, 9, 11, 12 of 6-E2-D5-2-C4-VH were at correct size (around 500 bp) so were sequenced. Clones 1-6, 9-12 of 6-E2-H5-2-A9-VL and clones 1-4, 6, 9-12 of 6-E2-H5-2-A9-VH were at correct size (around 500 bp) so were sequenced MW: molecular weight standard (DL2000)

FIG. 2. Assessment of anti-EPO (C4) efficacy on cell viability. The viability assay was performed on: FIG. 2A primary GSCs, FIG. 2B primary GECs, and FIG. 2C GBM-CSC cell line, FIG. 2D primary anaplastic astrocytoma cells, FIG. 2E DLD1, and FIG. 2F PC-3 alone or in combination with FTY720 or/with TMZ. Data are the mean±SD of at least 3 experiments in triplicate. *P<0.05; **P<0.01; ***P<0.001 versus CTR for all treatments.

FIG. 3. Assessment of anti-EPO (C4) efficacy on cell viability. The viability assay was performed on: FIG. 3A LNCAP, FIG. 3B MCF-7, and FIG. 3C K562, FIG. 3D A2780, FIG. 3E A549, and FIG. 3F SHSY-5Y alone or in combination with FTY720. Data are the mean±SD of at least 3 experiments in triplicate. *P<0.05; **P<0.01 versus CTR for all treatments.

FIG. 4. Assessment of anti-EPO (C4) efficacy on cell viability. The viability assay was performed on human healthy astrocytes alone or in combination with FTY720 or/with TMZ. Data are the mean±SD of at least 3 experiments in triplicate.

FIG. 5. Assessment of anti-EPO (C4) efficacy on cell viability. The viability assay was performed on: FIG. 5A primary GSCs, FIG. 5B primary GECs, and FIG. 5C GBM-CSC cell line, FIG. 5D primary anaplastic astrocytoma cells, FIG. 5E DLD1, and FIG. 5F PC-3 alone or in combination with anti-EPHB4 or FTY720 or/with TMZ. Data are the mean±SD of at least 3 experiments in triplicate. *P<0.05; **P<0.01; ***P<0.001 versus CTR for all treatments.

FIG. 6. Assessment of anti-EPO (C4) efficacy on cell viability. The viability assay was performed on: FIG. 6A LNCAP, FIG. 6B MCF-7, and FIG. 6C K562, FIG. 6D A2780, FIG. 6E A549, and FIG. 6F SHSY-5Y alone or in combination with anti-EPHB4 or FTY720. Data are the mean±SD of at least 3 experiments in triplicate. *P<0.05; **P<0.01 versus CTR for all treatments.

FIG. 7. Assessment of anti-EPO (C4) efficacy on cell viability. The viability assay was performed on human healthy astrocytes alone or in combination with anti-EPHB4, or/with FTY720. Data are the mean±SD of at least 3 experiments in triplicate.

FIG. 8. Assessment of anti-EPO (C4) efficacy in inhibiting angiogenesis in primary GECs. Representative images of tube-like formation assay performed on GECs cultured in Matrigel for 48 h in: FIG. 8A basal condition (CTR), FIG. 8B anti-EPO (C4), FIG. 8C TMZ, FIG. 8D FTY720, FIG. 8E anti-EPO (C4)+TMZ+FTY720. In FIG. 8F were reported the total tube length formed in the assay, measured using the Angiogenesis Analyzer plugin in ImageJ. Data are the mean±SD of at least 3 experiments in triplicate. * P<0.05 **P<0.01 versus CTR for all treatments.

FIG. 9. Assessment of anti-EPO (C4) efficacy in inhibiting GEC migration. Primary GECs were cultured on Ibidi Culture-Insert for 48 h in the following conditions: FIG. 9A basal condition (CTR), FIG. 9B anti-EPO (C4), FIG. 9C TMZ, FIG. 9D FTY720, FIG. 9E anti-EPO (C4)+TMZ+FTY720. The dotted white lines indicate the margin of the scratch, 500 μm wide. In FIG. 9F were reported the total number of migrated cells, counted using the Analyze Particle plugin in ImageJ. Data are the mean±SD of at least 3 experiments in triplicate. *P<0.05; **P<0.01 versus CTR for all treatments.

FIG. 10. Assessment of anti-EPO (C4) and anti-EPHB4 efficacy in inhibiting GEC migration. Primary GECs were cultured on Ibidi Culture-Insert for 48 h in the following conditions: FIG. 9A basal condition (CTR), FIG. 9B anti-EPO (C4), FIG. 9C anti-EPHB4, FIG. 9D anti-EPO (C4)+anti-EPHB4, FIG. 9E anti-EPO (C4)+anti-EPHB4+TMZ+FTY720. The dotted white lines indicate the margin of the scratch, 500 μm wide. In FIG. 9F were reported the total number of migrated cells, counted using the Analyze Particle plugin in ImageJ. Data are the mean±SD of at least 3 experiments in triplicate. *P<0.05; **P<0.01 versus CTR for all treatments.

FIG. 11. Assessment of anti-EPO (C4) efficacy in inhibiting DLD1 migration. Primary DLD1 were cultured on Ibidi Culture-Insert for 48 h in the following conditions: FIG. 11A basal condition (CTR), FIG. 11B anti-EPO (C4), FIG. 11C FTY720, FIG. 11D anti-EPO (C4)+FTY720. The dotted white lines indicate the margin of the scratch, 500 μm wide. In FIG. 11E were reported the total number of migrated cells, counted using the Analyze Particle plugin in ImageJ. Data are the mean±SD of at least 3 experiments in triplicate. *P<0.05 versus CTR for all treatments.

FIG. 12. Assessment of anti-EPO (C4) efficacy in inhibiting DLD1 migration. Primary DLD1 were cultured on Ibidi Culture-Insert for 48 h in the following conditions: FIG. 12A basal condition (CTR), FIG. 12B anti-EPO (C4), FIG. 12C anti-EPHB4, FIG. 12D anti-EPO (C4)+anti-EPHB4, FIG. 12E anti-EPO (C4)+anti-EPHB4+FTY720. The dotted white lines indicate the margin of the scratch, 500 μm wide. In FIG. 12F were reported the total number of migrated cells, counted using the Analyze Particle plugin in ImageJ. Data are the mean±SD of at least 3 experiments in triplicate. *P<0.05 versus CTR for all treatments.

FIG. 13. Assessment of anti-EPO (C4) efficacy on cell apoptosis. The analysis was performed by the assessment of Caspase activation on: FIG. 13A primary GSCs, FIG. 13B primary GECs, 13 GBM-CSC line; FIG. 13D primary anaplastic astrocytoma cells, FIG. 13E DLD1, and FIG. 13F PC-3 with anti-EPO (C4) alone or in combination with FTY720 or/with TMZ. Data are the mean±SD of at least 3 experiments in triplicate. *P<0.05 versus CTR for all treatments.

FIG. 14. Assessment of anti-EPO (C4) efficacy on cell apoptosis. The viability assays were performed on: FIG. 14A LNCAP, FIG. 14B MCF-7, and FIG. 14C K562, FIG. 14D A2780, FIG. 14E A549, and FIG. 14F SHSY-5Y with anti-EPO (C4) alone or in combination with FTY720. Data are the mean±SD of at least 3 experiments in triplicate. *P<0.05 versus CTR for all treatments.

FIG. 15. Neuroinflammatory disease model: analysis of cell viability and proliferation of microglia: N9 microglia cells, cultured in the presence of lipopolysaccharide (LPS), as potent inflammatory stimulus, were used and subjected to treatment with monoclonal anti-EPO (C4) antibody+FTY720. The effects on cell viability (FIG. 15A) and cell proliferation (FIG. 15B) of activated microglia were assessed. In addition morphology analysis was performed on microglial after treatment with CTR (FIG. 15C), LPS (FIG. 15D), LPS+anti-EPO (C4) (FIG. 15E), LPS+anti-EPO (C4)+FTY720 (FIG. 15F). Data are the mean±SD of at least 3 experiments in triplicate. #P<0.05 versus CTR; *P<0.05 versus CTR+LPS for all treatments.

FIG. 16. Neuroinflammatory disease model: analysis of microglia migration: N9 microglia cells, cultured in the presence of lipopolysaccharide (LPS), as potent inflammatory stimulus, were used and subjected to treatment with monoclonal anti-EPO (C4) antibody+FTY720. The effects on cell migration was assessed after the following treatments: CTR (FIG. 16A), LPS (FIG. 16B), LPS+anti-EPO (C4) (FIG. 16C), LPS+FTY720 (FIG. 16D), LPS+anti-EPO (C4)+FTY720 (FIG. 16E). In FIG. 16F were reported the total number of migrated cells, counted using the Analyze Particle plugin in ImageJ. Data are the mean±SD of at least 3 experiments in triplicate. #P<0.05 versus CTR; *P<0.05; **P<0.01 versus CTR+LPS for all treatments.

FIG. 17. Neuroinflammatory disease model: analysis of cell viability and proliferation of microglia. Activated N9 microglia cells, cultured in the presence of LPS, were used to test the effect of anti-EPO (C4) treatment on viability (FIG. 17A) and proliferation (FIG. 17B) after the following treatments: CTR, LPS, LPS+anti-EPO (C4), LPS+anti-EPHB4, LPS+anti-EPO (C4)+anti-EPHB4+FTY720. Data are the mean±SD of at least 3 experiments in triplicate. #P<0.05 versus CTR; **P<0.01 versus CTR+LPS for all treatments.

FIG. 18. Inflammatory and proliferative disease model: analysis of cell viability and proliferation on synovial endothelial cells (S-ECs) from haemophilic patient. S-ECs were cultured under the following treatments: CTR, anti-EPO (C4), FTY720, anti-EPO (C4)+FTY720 and cell viability (FIG. 18A) and proliferation (FIG. 18B) were assessed. **P<0.01, ***P<0.001 versus CTR for all treatments.

FIG. 19. Assessment of anti-EPO (C4) efficacy in inhibiting angiogenesis in synovial endothelial cells (S-ECs) from haemophilic patient. Representative images of tube-like formation assay performed on S-ECs cultured in Matrigel for 48 h in: FIG. 19A basal condition (CTR), FIG. 19B anti-EPO-(C4), FIG. 19C FTY720, FIG. 19D anti-EPO (C4)+FTY720. In FIG. 19E were reported the total tube length formed in the assay, measured using the Angiogenesis Analyzer plugin in ImageJ. Data are the mean±SD of at least 3 experiments in triplicate. * P<0.05 versus CTR for all treatments.

FIG. 20. Inflammatory and proliferative disease model: analysis of S1PR1 expression on synovial endothelial cells (S-ECs) from haemophilic patient. S1 PR1 expression was assessed on endothelial cells isolated from the S-ECs of hemophilic patients after the following treatments: FIG. 20A CTR, FIG. 20B anti-EPO (C4), FIG. 20C FTY720, FIG. 20D anti-EPO (C4)+FTY720.

FIG. 21. Inflammatory and proliferative disease model: analysis of cell viability and proliferation on synovial endothelial cells (S-ECs) from haemophilic patient. S-ECs were cultured under the following treatments: CTR, anti-EPO (C4), anti-EPHB4, anti-EPO (C4)+anti-EPHB4, anti-EPO (C4)+anti-EPHB4+FTY720. Cell viability (FIG. 21A) and proliferation (FIG. 21B) were assessed. *P<0.05; **P<0.01 versus CTR for all treatments.

FIG. 22. Assessment of anti-EPO (C4) efficacy on cell viability. FIG. 22A, representative images captured by microscope of HOC84 stem cells after treatment with anti-EPO (C4) alone or in combination with FTY720. The viability assay was performed on: HOC84 FIG. 22B treated with anti-EPO (C4) alone or in combination with FTY720 and FIG. 22C treated with anti-EPO (C4) alone or in combination with anti-EPHB4 or/and FTY720. Data are the mean±SD of at least 3 experiments in triplicate. *P<0.05; **P<0.01; versus CTR for all treatments.

FIG. 23. anti-EPO (C4) treatment modulates gene expression profile of HOC84 stem cells. The analysis was performed by the assessment of gene expression by RealTime PCR on genes related to FIG. 23A apoptosis, FIG. 23B proliferation, FIG. 23C angiogenesis, and FIG. 23D inflammation. Data are the mean±SD of at least 3 experiments in triplicate. *P<0.05, **P<0.01 versus CTR for all treatments.

FIG. 24. anti-EPO (C4) treatment sensitizes chemo-resistant cancer stem cells to anti-tumor treatment. The viability assay was performed on: FIG. 24A primary TMZ-resistant glioblastoma stem cells (GSC-R), FIG. 24B TMZ-resistant U87 (U87-R), FIG. 24C HOC84 stem cells treated in: CTR condition, anti-EPO (C4), TMZ at 100 μM, and anti-EPO (C4)+TMZ. Data are the mean±SD of at least 3 experiments in triplicate. *P<0.05; **P<0.01; ***P<0.001 versus CTR for all treatments.

FIG. 25. Evaluation of human anti-EPO (C4) binding to hEPO by in-silico docking. Investigation of hEPO amino acid residues that participate in the antigen-antibody recognition. In FIG. 25A the most probable binding complex based on docking was reported; FIG. 25B showed binding site of the hEPO/EPOR complex. In FIG. 25C, in red, were reported the amino acid residues at the interface of the complex hEPO/anti-EPO (C4) and hEPO/EPOR

FIG. 26. Pharmacokinetic profile of anti-EPO (C4) in plasma and tissues performed by Surface Plasmonic Resonance analysis.

FIG. 26A SPR technology can allow to determine the plasma and tissue concentrations of antibody, on the basis of an appropriate calibration curve. FIG. 26B Blood samples were collected from the retroorbital plexus under isoflurane anesthesia at 4, 24, 48, 72, 120, and 168 h from treatment. The pharmacokinetic profile in plasma was studied, and the results showed that the anti-EPO (C4) was stable in circulation with a half-time of elimination of about 4.4 days after single administration. FIG. 26C showed the sensorgrams of the binding of human EPO (H-EPO) compared to murine EPO (mEPO). The concentration of the EPO variants were 10 μM in PBST, which was flown for 180 seconds over the immobilized anti-EPO, followed by 1600 seconds of dissociation. FIG. 26D showed pooled plasma, tumors, liver, and kidney from animals treated with anti-EPO(C4) at 10 mg/Kg by intravenous administration (IV) analyzed by SPR.

FIG. 27. In vivo administration. Absence of erythropoiesis impairment and hematological toxicity. Blood count of mice treated intravenously (IV) for 18 days with placebo, saline solution, and anti-EPO at 10 mg/Kg. (A) RBC: red blood cells; (B)HGB: hemoglobin; (C) HCT: hematocrit; (D) MCV: mean corpuscular volume; (E) MCH: mean corpuscular hemoglobin; (F) MCHC: mean corpuscular hemoglobin concentration; (G) RDW: red cell distribution width; (H) RET: reticulocyte Count; (I) PLT: platelets. Data are means±SD. *p<0.05, **p<0.01 vs CTRL.

FIG. 28. In vivo administration. Absence of erythropoiesis impairment and hematological toxicity. Blood count of mice treated intravenously (IV) for 18 days with placebo, saline solution, and anti-EPO at 10 mg/Kg. (A) WBC: white blood cells; (B) NEUT: neutrophils; (C) LYMP: lymphocytes; (D) MONO: monocytes; (E) EOS: eosinophils; (F) BASO: basophils. Data are means±SD. *p<0.05, **p<0.01 vs CTRL.

FIG. 29. In vivo administration toxicity profile. Absence of renal and liver toxicity.

Sera were collected without anticoagulant. The samples were left at environment temperature for at least 30′ minutes, then centrifuged at 500 g for 10 min, the supernatants were collected, and analyzed for Biochemical tests. Biochemistry analysis was measured at two time points, 11 and 18 days after treatment starting, revealing no significant variations after anti-EPO administration at different doses and at different timepoint. FIG. 29A Urea; FIG. 29B Creatinine; FIG. 29C Albumine, FIG. 29D AST; aspartate aminotransferase; FIG. 29E ALT: alanine aminotransferase.

FIG. 30. Gene expression of cell derived xenograft GSCs after in vivo treatment with anti-EPO (C4). The analysis was performed by the assessment of gene expression profile on human tumor GSC-derived xenograft by RealTime PCR. The analysis was conducted on genes related to FIG. 30A apoptosis, FIG. 30B inflammation, FIG. 30C proliferation. Data are the mean±SD of at least 3 experiments in triplicate. *P<0.05, **P<0.01 versus CTR for all treatments.

FIG. 31. Diagnostic Use: Example of a multi-level panel for diagnostic use, consisting on the evaluation of the molecular signature of EPO: FIG. 31A analysis of copy number variation (CNV) in EPO-related cytoband 7q22.1; FIG. 31B. EPO and genes related to EPO signaling for gene expression evaluation; FIG. 31C. Analysis of EPO secreted by cancer cell models; FIG. 31D EPO expression in by Western Blot analysis. Data are the mean±SD of at least 3 experiments in triplicate.

### DETAILED DESCRIPTION OF THE INVENTION

The invention herein provides, isolated antibodies that bind to EPO and uses thereof. Pharmaceutical compositions as well as methods of treatment are also provided.

The techniques and procedures described or referenced herein are generally well understood and commonly employed using conventional methodology by those skilled in the art, such as, for example, the widely utilized hybridoma methodologies and phage display techniques.

The present invention concerns in a first aspect an isolated anti-Erythropoietin (EPO) antibody, also identified as “C4 antibody” or “C4”, wherein said antibody comprises:
a. a variable domain of a light chain (VL) having the amino acid sequence of SEQ ID NO:6; and
b. a variable domain of a heavy chain (VH) having the amino acid sequence of SEQ ID NO:14.

The hybridoma which produces the C4 antibody according to the present invention, said antibody comprising a variable domain of a light chain (VL) having the amino acid sequence of SEQ ID NO:6; and a variable domain of a heavy chain (VH) having the amino acid sequence of SEQ ID NO:14, was deposited at the Leibniz-Institute DSMZ with the accession number DSM ACC 3370 on 09.09.2021.

In a preferred aspect, the antibody of the present invention is an isolated anti-EPO antibody, wherein said antibody comprises 6 CDR regions, said CDR regions being:
a. a VL-CDR1 having the amino acid sequence of SEQ ID NO:4;
b. a VL-CDR2 having the amino acid sequence of GAS (Gly-Ala-Ser);
c. a VL-CDR3 having the amino acid sequence of SEQ ID NO:5
d. a VH-CDR1 having the amino acid sequence of SEQ ID NO:11;
e. a VH-CDR2 having the amino acid sequence of SEQ ID NO:12; and
f. a VH-CDR3 having the amino acid sequence of SEQ ID NO:13.

For the purposes of the present disclosure, each sequence has a corresponding SEQ ID NO. as follows:
SEQ ID NO:1 corresponds to the DNA sequence of the CDR1 region of the variable light chain of the anti-EPO antibody (VL-CDR1):
GAAAGTGTTGACTATTATGGCACAGGTTTA
GGTGCATCC corresponds to the DNA sequence of the CDR2 region of the variable light chain of the anti-EPO antibody (VL-CDR2)
SEQ ID NO:2 corresponds to the DNA sequence of the CDR3 region of the variable light chain of the anti-EPO antibody (VL-CDR3):
CAGCAAACTAGGAAGGTTCCTTCGACG
SEQ ID NO:3 variable light chain DNA sequence (333 bp, CDRs in bold: FR1-CDR1-FR2-CDR2-FR3-CDR3-FR4):
GATATCGTTCTCACTCAATCTCCAGCTTCTTTGGCTGTGTCTCTAGGGCAGAGAG CCACCATCTCCTGCAGAGCCAGTGAAAGTGTTGACTATTATGGCACAGGTTTAA TGCAGTGGTACCAACAGAGACCAGGACAGCCACCCAAACTCCTCATCTATGGTG CATCCAACGTAGGATCTGGGGTCCCTGCCAGGTTTAGCGGCAGTGGGTCTGGG ACAGACTTCAGCCTCAACATCCATCCTGTGGAGGGGGATGATATTGCAATGTAT TTCTGTCAGCAAACTAGGAAGGTTCCTTCGACGTTCGGTGGAGGCACCAAGTT GGAAATCAAA
SEQ ID NO:4 corresponds to the amino acid sequence of the CDR1 region of the variable light chain of the anti-EPO antibody (VL-CDR1): ESVDYYGTGL GAS (Gly-Ala-Ser) corresponds to the amino acid sequence of the CDR2 region of the variable light chain of the anti-EPO antibody (VL-CDR2)
SEQ ID NO:5 corresponds to the amino acid sequence of the CDR3 region of the variable light chain of the anti-EPO antibody (VL-CDR3): QQTRKVPST
SEQ ID NO: 6 variable light chain amino acid sequence (111 aa, CDRs in yellow: FR1-CDR1-FR2-CDR2-FR3-CDR3-FR4): DIVLTQSPASLAVSLGQRATISCRASESVDYYGTGLMQWYQQRPGQPPKLLIYGAS NVGSGVPARFSGSGSGTDFSLNIHPVEGDDIAMYFCQQTRKVPSTFGGGTKLEIK
SEQ ID NO:7 corresponds to the DNA sequence of the CDR1 region of the variable heavy chain of the anti-EPO antibody (VH-CDR1): GGATTCACTTTCAGTACCTATACC
SEQ ID NO:8 corresponds to the DNA sequence of the CDR2 region of the variable heavy chain of the anti-EPO antibody (VH-CDR2): ATTAGTAATGGTGGTGATAGAACC
SEQ ID NO:9 corresponds to the DNA sequence of the CDR3 region of the variable heavy chain of the anti-EPO antibody (VH-CDR3): GCAAGACATAATATTACTACGGTTCCCTTTACTATGGACTAC
SEQ ID NO:10 variable heavy chain DNA sequence (363 bp, CDRs in bold: FR1-CDR1-FR2-CDR2-FR3-CDR3-FR4): GAGGTGAAGCTGCAGGAGTCTGGGGGAGGTTTAGTGCAGCCTGGAGGGTCCCT GAAACTCTCCTGTGCAGCCTCTGGATTCACTTTCAGTACCTATACCATGTCTTG GGTTCGCCAGACTCCAGAGAAGAGGCTGGAGTGGGTCGCATACATTAGTAATG GTGGTGATAGAACCTACTATCCAGACACTGTAAAGGGCCGATTCACCATCTCCA GAGACGATGCCAAGAACACCCTGTTCCTGCAAATGAGCAGTCTGAAGTCTGAGG ACACGGCCATGTATTACTGTGCAAGACATAATATTACTACGGTTCCCTTTACTAT GGACTACTGGGGTCAAGGAACCTCAGTCACCGTCTCCTCA
SEQ ID NO:11 corresponds to the amino acid sequence of the CDR1 region of the variable heavy chain of the anti-EPO antibody (VH-CDR1): GFTFSTYT
SEQ ID NO:12 corresponds to the amino acid sequence of the CDR2 region of the variable heavy chain of the anti-EPO antibody (VH-CDR2): ISNGGDRT
SEQ ID NO:13 corresponds to the amino acid sequence of the CDR3 region of the variable heavy chain of the anti-EPO antibody (VH-CDR3): ARHNITTVPFTMDY
SEQ ID NO:14 variable heavy chain amino acid sequence (VH) (121 aa, CDRs in bold: FR1-CDR1-FR2-CDR2-FR3-CDR3-FR4): EVKLQESGGGLVQPGGSLKLSCAASGFTFSTYTMSWVRQTPEKRLEWVAYISNGG DRTYYPDTVKGRFTISRDDAKNTLFLQMSSLKSEDTAMYYCARHNITTVPFTMDYW GQGTSVTVSS
SEQ ID NO:15: EPO amino acid sequence (N-terminal signal peptide+protein chain) aa 1-193 MGVHECPAWLWLLLSLLSLPLGLPVLGAPPRLICDSRVLERYLLEAKEAENITTGCAE HCSLNENITVPDTKVNFYAWKRMEVGQQAVEVWQGLALLSEAVLRGQALLVNSSQ PWEPLQLHVDKAVSGLRSLTTLLRALGAQKEAISPPDAASAAPLRTITADTFRKLFRV YSNFLRGKLKLYTGEACRTGDR
SEQ ID NO:16 EPO mature peptide amino acid sequence (aa 28-193) APPRLICDSRVLERYLLEAKEAENITTGCAEHCSLNENITVPDTKVNFYAWKRMEVG QQAVEVWQGLALLSEAVLRGQALLVNSSQPWEPLQLHVDKAVSGLRSLTTLLRALG AQKEAISPPDAASAAPLRTITADTFRKLFRVYSNFLRGKLKLYTGEACRTGDR
SEQ ID NO: 17 EPO gene sequence.

For the purposes of the present invention, the “antibody” or “monoclonal antibody” is a “negative functional modulator of EPO” against human EPO. In particular the antibody is against the mature form of EPO which corresponds to amino acids (AA) 28-193 of the whole EPO amino acid sequence (SEQ ID NO:15). The mature EPO amino acid sequence (AA 28-193) is described in SEQ ID NO:16. Human EPO is encoded by the gene sequence SEQ ID NO: 17.

The anti-EPO antibody is a molecule capable of recognizing and binding an amino acid sequence included in Erythropoietin, capable of direct or indirect interaction with EPO, and/or direct or indirect interaction with the biosynthetic pathway of EPO, wherein said interactions have resulted in a decrease in the levels of EPO, rather than a decrease in the stimulation of the signal transduction cascade in which EPO is involved. In a further embodiment, said negative functional modulators of EPO act on EPO which has undergone post-translational modifications.

In a more preferred aspect, the isolated anti-EPO antibody of the invention is a monoclonal antibody, a chimeric antibody and/or is humanized or human, is an antibody fragment selected from a Fab, Fab′-SH, Fv, scFv, or (Fab′)2fragment and more preferably further comprises a framework sequence and at least a portion of the framework sequence is a human consensus framework sequence.

The main objective of humanization process is to reduce antibodies immunogenicity in order to improve tolerance in humans and improve their biophysical properties. Briefly, variable regions sequences information is generated by Reverse Transcription of total RNA extraction obtained from hybridoma cell line. Variable regions of the heavy (VH) and light chains (VL) are amplified by PCR and cloned into shuttle vector for sequencing. A total of 5 independent clones are sequenced for each variable chain. Sequences of the hybridoma are determined from the sequencing results of the VH and VL. A chimeric construct is designed and expressed by combination of mouse VH and VL variable regions with human IgG1 constant regions in order to confirm affinity/binding and biological function related to the parental mouse hybridoma. Antibody sequences are humanized by grafting the three CDRs from the light chain variable region (VL) into human VL germlines which are as homologous as possible to the mouse antibody VL. Similarly, the three CDRs from the heavy chain variable region (VH) are grafted into human VH germlines which are as homologous as possible to the mouse antibody VH. In addition, because different framework context might bring added value to the resulting antibody, CDRs are grafted into human VH and VL germlines which are well-known to exhibit good overall biophysical properties even if they are less homologous. A total of 9-18 VH/VL combinations are generated between the CDR-grafted VH, the CDR-grafted VL, and the chimeric versions of both VH and VL. Proof of concept quantities of each recombinant humanized antibody are transiently produced through XtenCHO™ platform and evaluated for binding/biological activity/biophysical properties compared to the chimeric version of parental mouse hybridoma. Comprehensive antibody affinity maturation services can be done via phage display using custom libraries generated by random or target mutagenesis. The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention.

In addition, antibodies may be prepared by different techniques. For example, monoclonal antibodies may be purified from cells that naturally express them, such as hybridoma cells, or produced in recombinant expression system both from mammalian system or prokaryotes (e.g. Escherichia Coli). More recently, fragment antibodies have been introduced in clinical practice. Indeed, fragment antibodies are emerging as great tools in imaging and diagnostics because they are capable of detecting cellular proteins with high affinity and specificity. Antibody fragments include, but not limited to: Fab, F(ab′)2, single chain antibodies, nanobodies, diabodies, triabodies, tetrabodies, and domain antibodies. They can be easily linked to radioisotopes, fluorescent molecules or enzymes that tag specific biomarkers in patients. They also have a shorter half-life in the body which results in faster clearance and may result in fewer risks of side effects from potentially invasive diagnostic agents. Where desired the affinity of the monoclonal antibody or fragment antibody according to the invention, containing one or more of CDRs above-mentioned, can be improved by affinity maturation procedures.

Preferably the antibody herein described is a full-length monoclonal antibody and is a bispecific anti-EPO antibody. The anti-EPO antibody has an amino acid sequence identical to or comprising 0, 1, 2, or 3 amino acid residue substitutions relative to the VL of SEQ ID NO:6 and to the VH of SEQ ID NO:14.

The advantageous properties of the antibody of the present invention (C4) will be apparent in the experimental section.

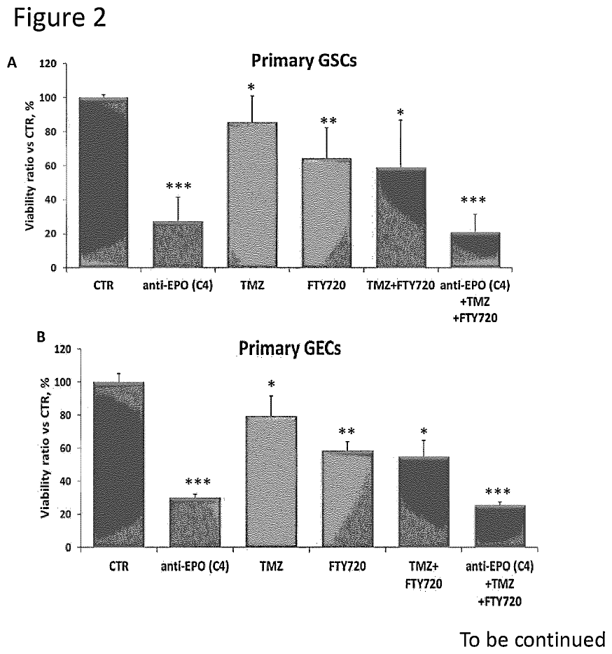

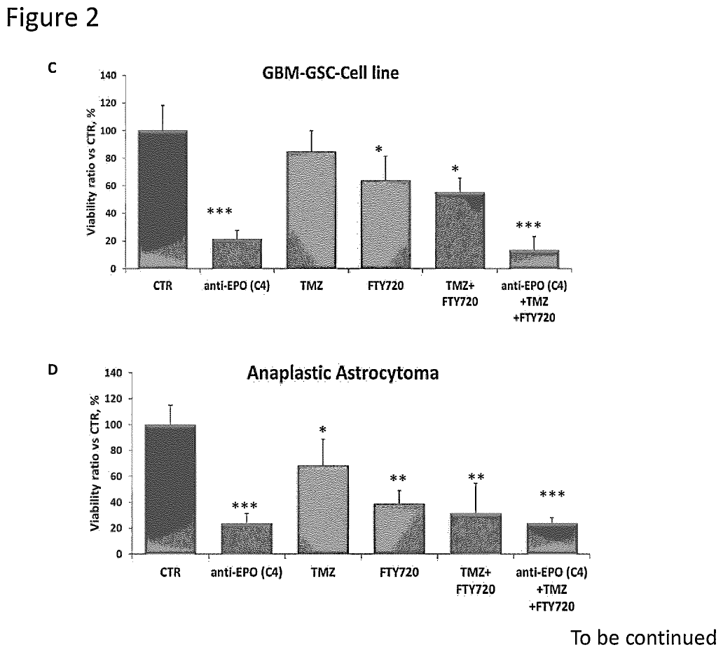

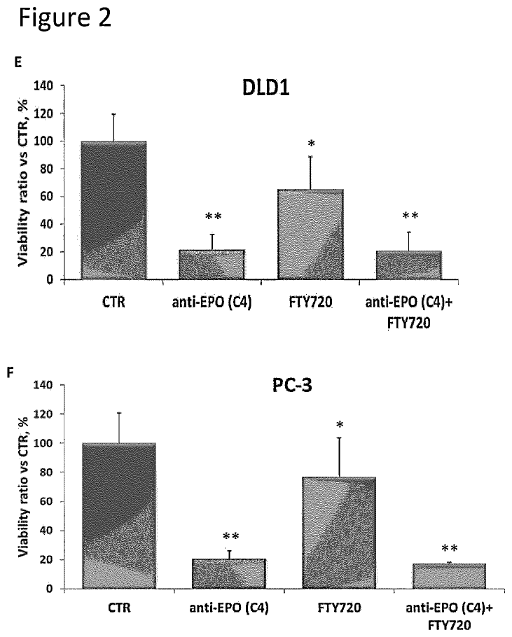

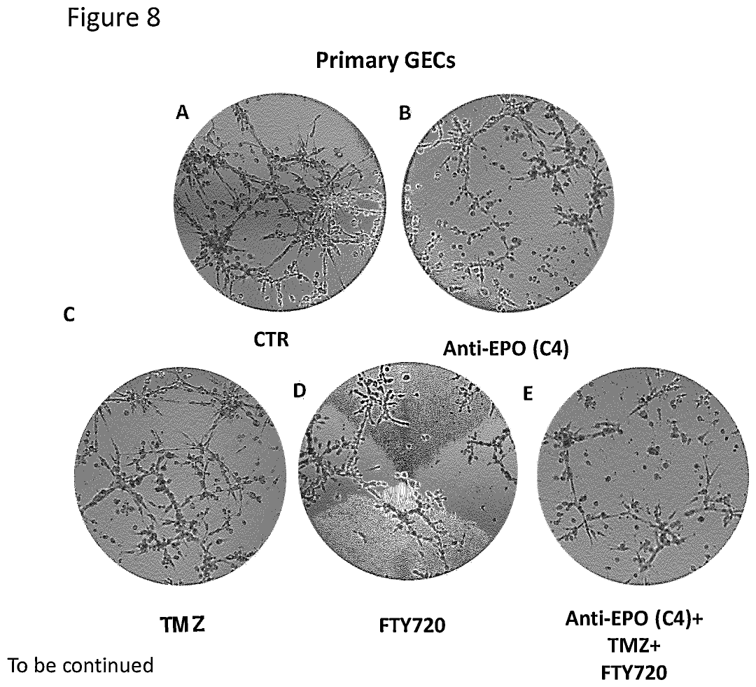

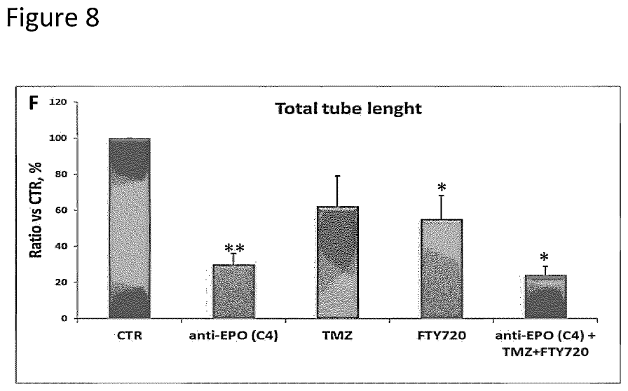

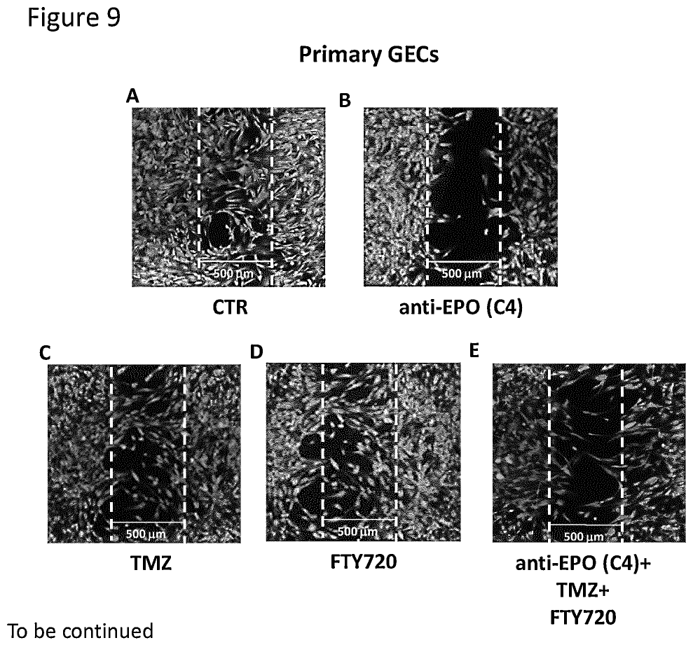

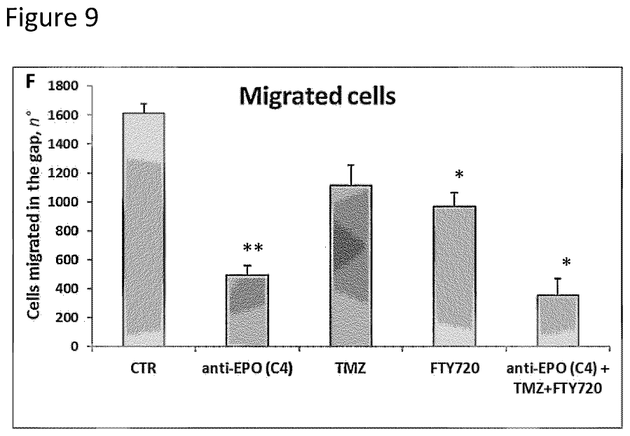

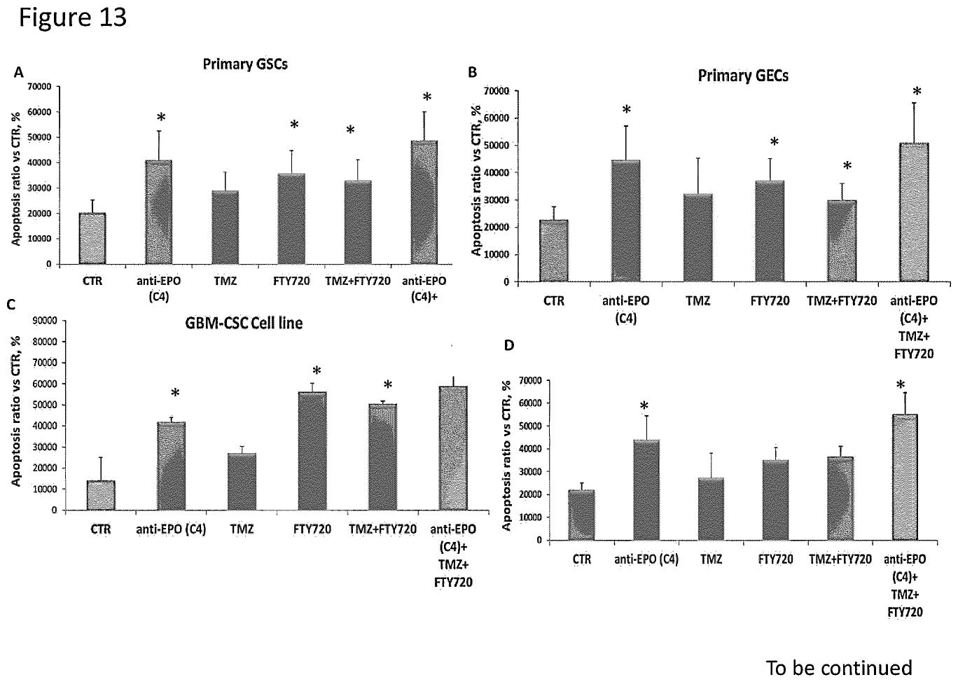

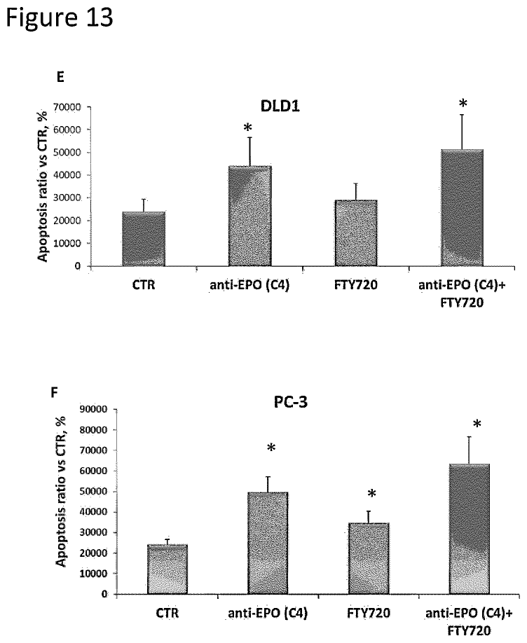

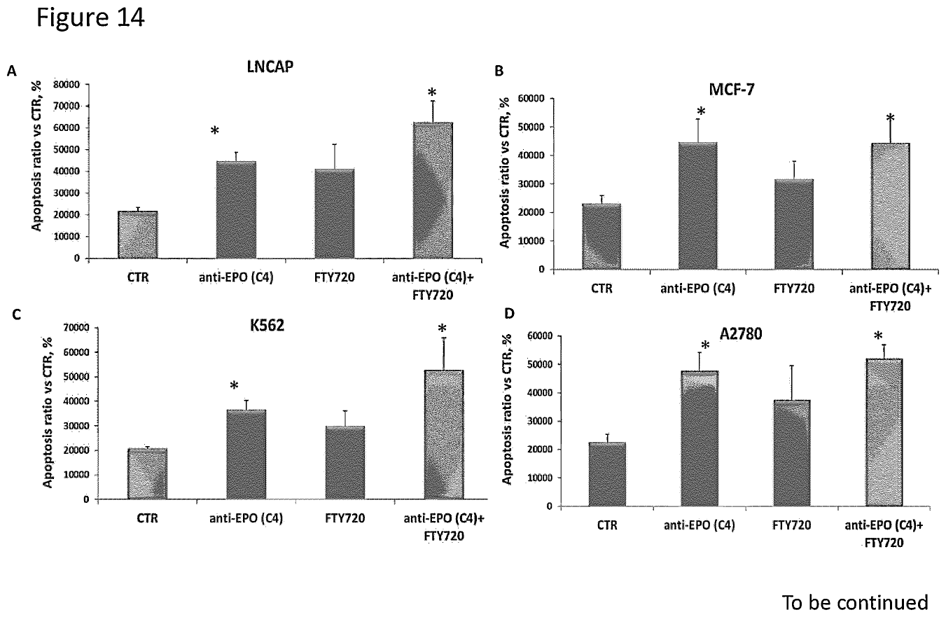

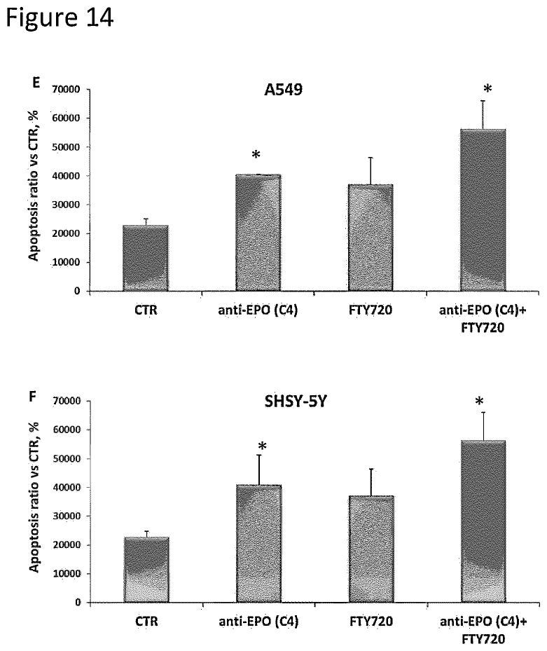

Anti-EPO (C4) was able to inhibit proliferation for more 70% in human glioblastoma stem cells, in tumor endothelial cells, in cancer cells derived from anaplastic astrocytomas, colon cancer, prostate cancer, breast cancer, leukemia, ovarian cancer, and lung cancer (Example 2, FIGS. 2,3). In addition, anti-EPO (C4) was able to significantly inhibit formation of tube-like structures (Example 4, FIG. 8), inhibit cell tumor migration (Examples 5 and 6, FIGS. 9-12) increase apoptosis (Example 7, FIGS. 13, 14).

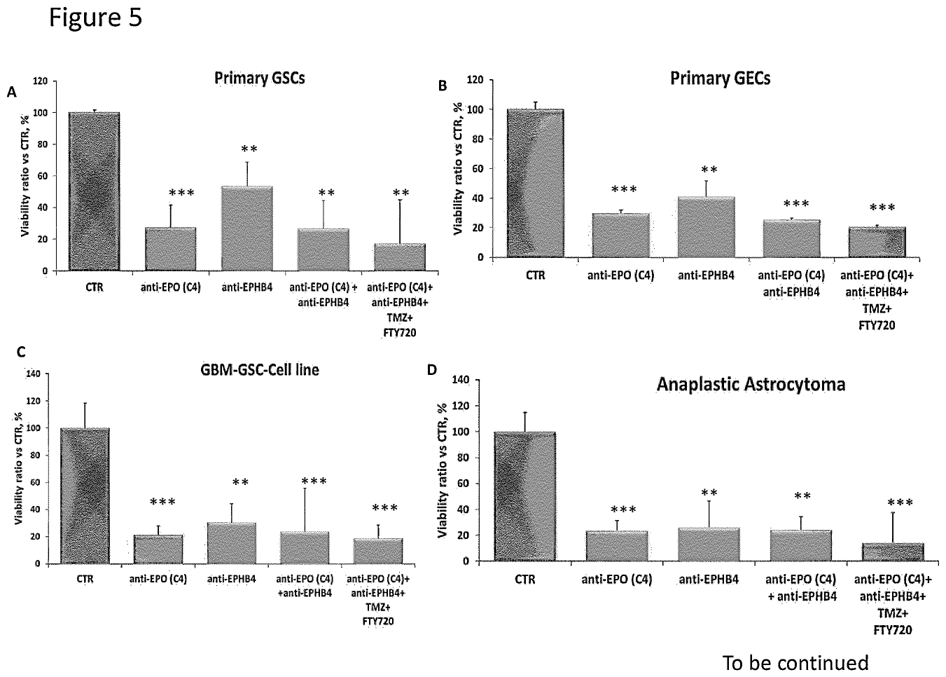

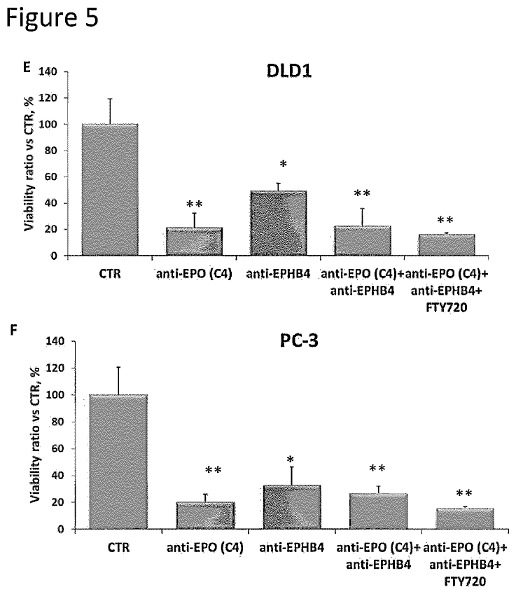

Notably, anti-EPO (C4) acquired a higher neutralizing capacity when co-administered with anti-EPHB4, an EPO receptor, mediating non-erythropoietic functions (Examples 8-11, FIGS. 5, 6, 10, 12).

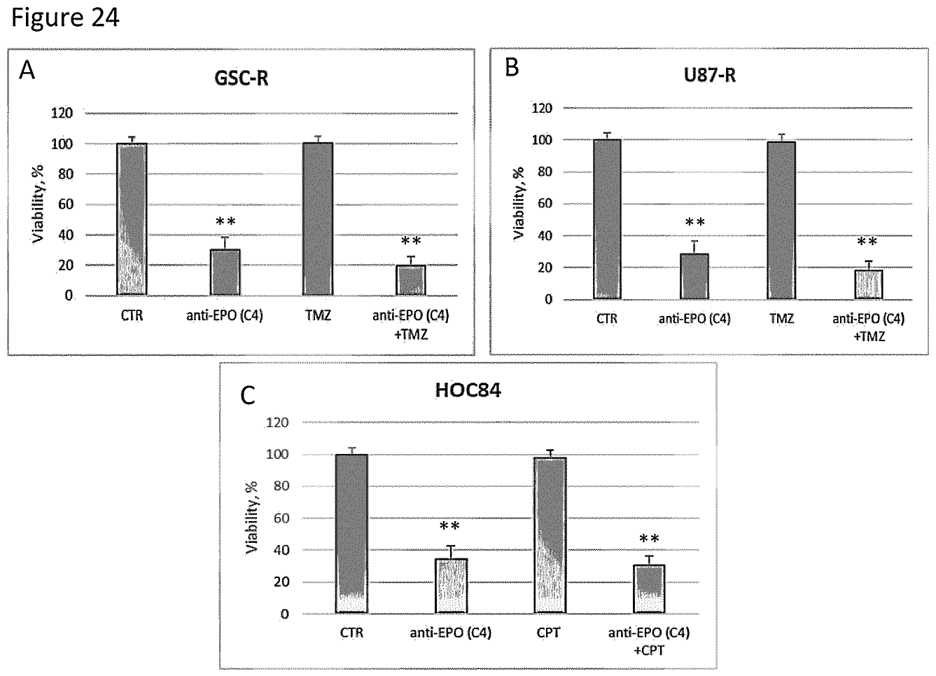

Interestingly, the anti-tumoral effects were potentiated by the co-treatment of anti-EPO (C4) with FTY720, and/or TMZ. These results were more evident when chemo-resistant tumors, such as glioblastoma and ovarian cancer, were treated in co-administration with the chemotherapeutic current standard treatment (Temozolomide for brain tumors, carboplatin for ovarian cancer) (Example 17, FIG. 24)

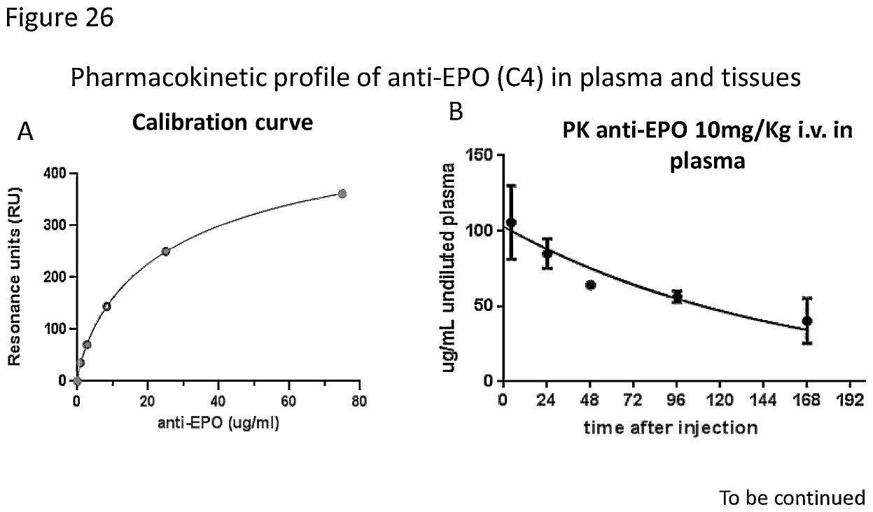

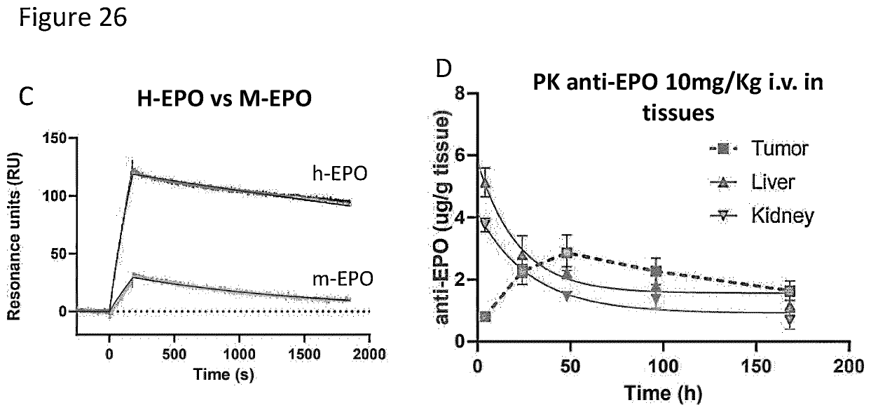

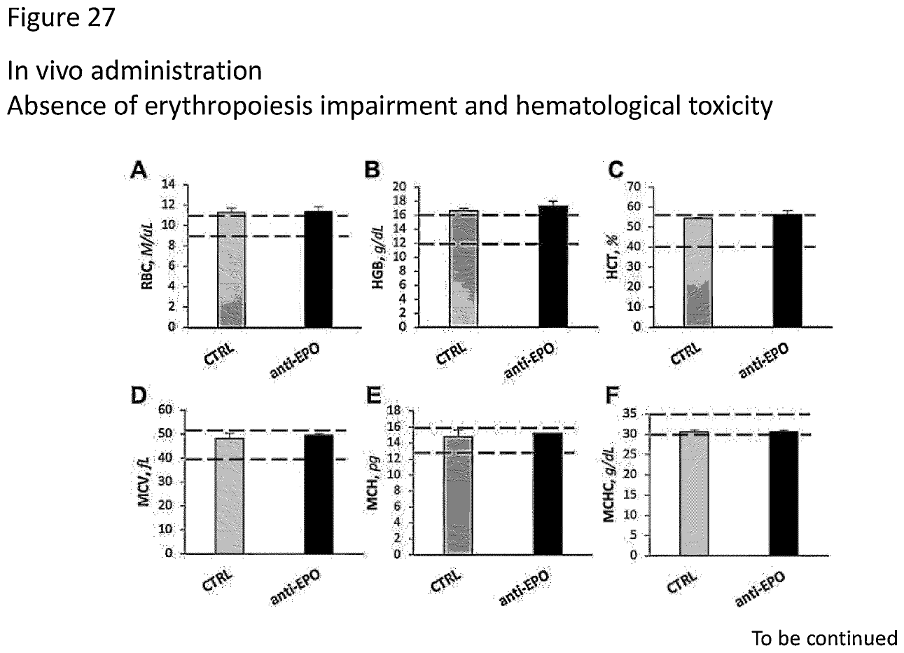

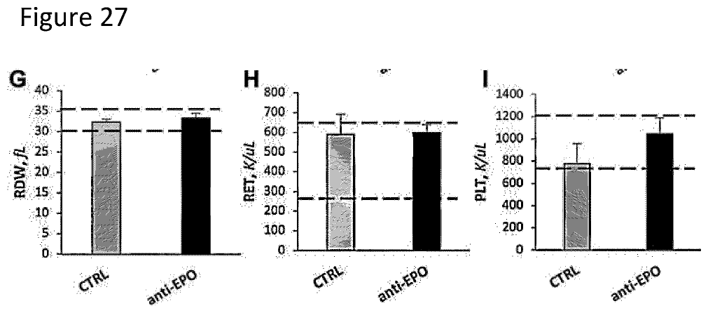

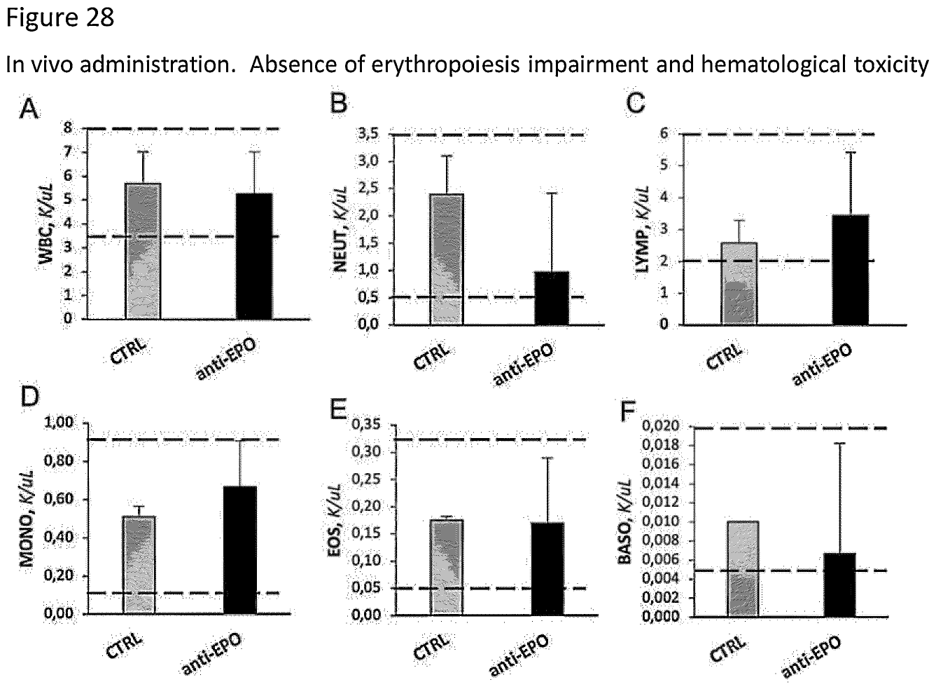

In particular, antibody C4 has been seen to have:
good stability in the bloodstream: as described in Example 19 and FIG. 26, the antibody of the present invention demonstrated that anti-EPO (C4) was stable in circulation with a half-time of elimination of about 4.4 days after single administration
a high affinity for human EPO (h-EPO). The affinity of C4 is 20 times higher compared to murine EPO (m-EPO). This can be seen in Example 19 and FIG. 26 where the representative sensorgrams (FIG. 26C) clearly revealed differences in their affinity. Indeed, h-Epo has an affinity 20 times higher than m-Epo (KD=1000 nM vs 50 nM).
improved uptake of the antibody of the invention in tumor tissues, when compared to liver and kidney absorption as can be seen Example 19 and Figure X26D demonstrating a good penetration efficacy within tumor tissues.
no influence on hematopoiesis: as can be seen in the results shown in Example 20 and FIGS. 27, 28, following in vivo administration of the C4 antibody, there is an absence of erythropoiesis impairment and hematological toxicity.
no renal toxicity. The C4 antibody has been tested to verify toxicity on kidneys.

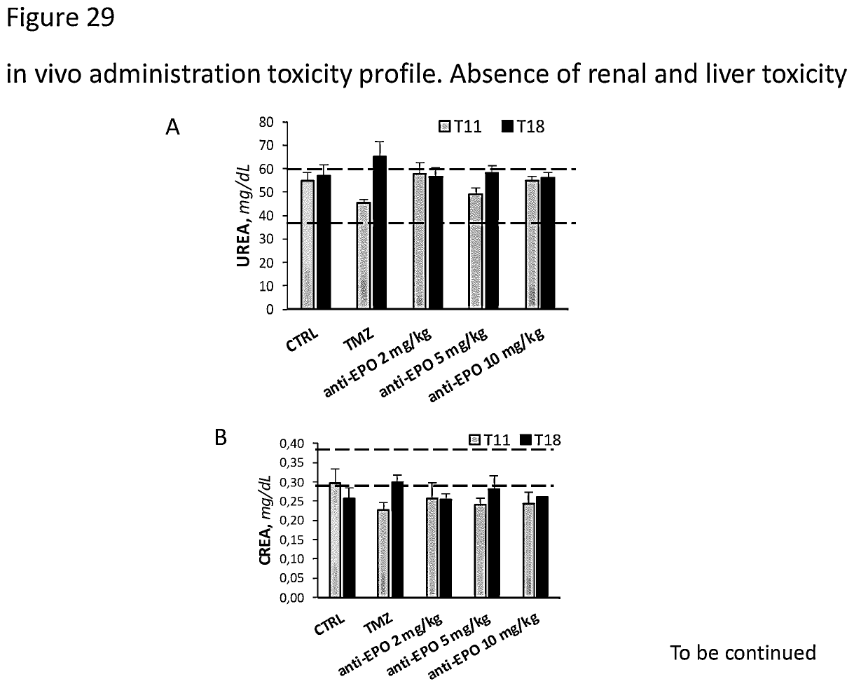

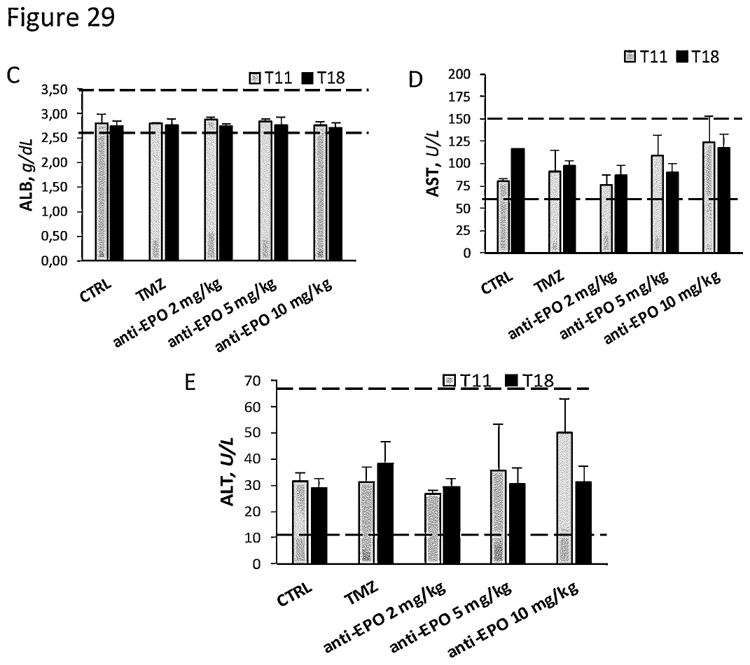

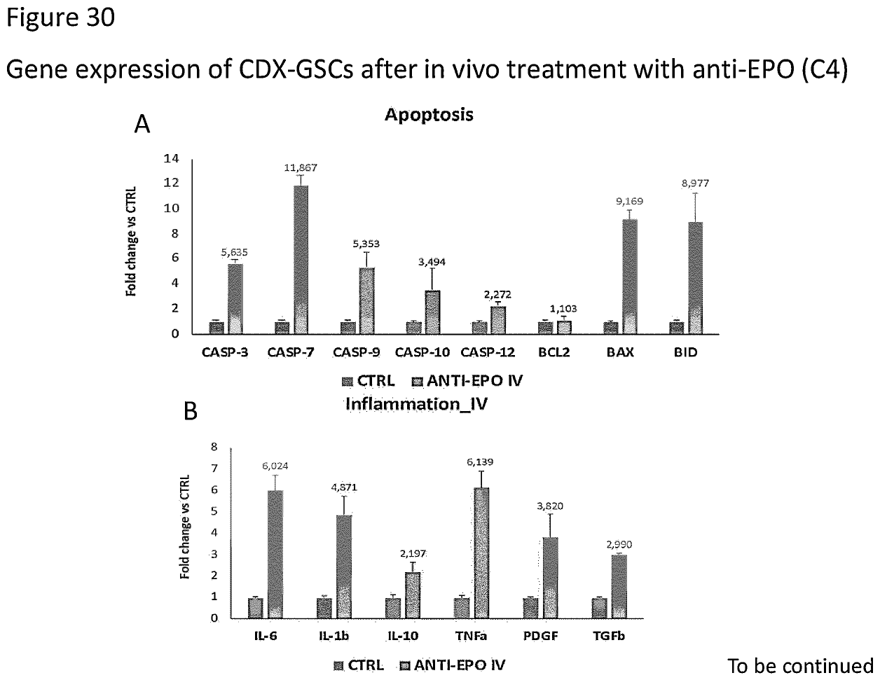

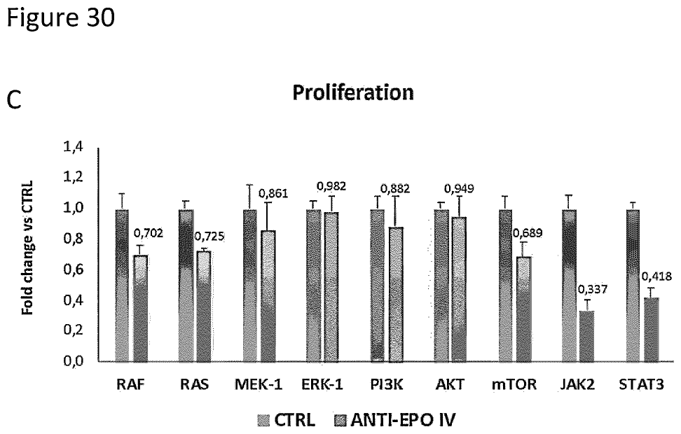

As can be seen in Example 20 and FIG. 29A, 29B.
no hepatic toxicity: as described in Example 20 and FIG. 29C-E, the antibody of the present invention demonstrated no alteration of the biochemical parameters analyzed in blood sample of animals treated with placebo or anti-EPO (C4) following 11 and 18 days of treatment.
evidence for modulation of gene expression in xenografted glioblastoma cancer stem cells following anti-EPO (C4) in vivo administration, as reported in Example 21, FIG. 30A-C.

Bispecific antibodies are monoclonal antibodies that have binding specificities for at least two different antigens. In certain embodiments, bispecific antibodies are human or humanized antibodies. In certain embodiments, one of the binding specificities is for EPO and the other is for any other antigen. In certain embodiments, bispecific antibodies may bind to two different epitopes of EPO. Bispecific antibodies may also be used to localize cytotoxic agents to cells which express EPO receptors.

In a second aspect, herein described is a polynucleotide encoding the anti-EPO antibody according to the present invention, having the VL sequence of SEQ ID NO:3 and the VH sequence of SEQ ID NO:10.

In a further aspect, the invention provides for a vector comprising the polynucleotide encoding the anti-EPO antibody, wherein the vector is optionally an expression vector.

In a still further aspect, described herein is a host cell comprising the vector of the present invention, preferably the host cell is prokaryotic, eukaryotic, or mammalian.

In a fifth aspect, the invention provides for an immunoconjugate comprising the anti-EPO antibody of the present invention conjugated to an agent, such as a drug or cytotoxic agent (e.g. a chemotherapeutic compound, a biological antibody, an anti-tumoral drugs) or co-administered in combination with a negative functional modulator of S1 P signaling, and/or anti-EPO receptors (e.g. EPOR, EPHB4, CSF2RB), and/or EPO mimetics.

A further aspect of the invention describes a method for producing the anti-EPO antibody of the invention or the immunoconjugate comprising said anti-EPO antibody, said method comprising (a) expressing the vector comprising the polynucleotide encoding the anti-EPO antibody in a suitable host cell, preferably a prokaryotic or eukaryotic host cell and (b) recovering the antibody or immunoconjugate.

In another embodiment the nucleotide sequence of the antibodies of the present invention, encoding the corresponding amino acid sequences of the anti-EPO antibodies can be modified, for example by random or site-directed mutagenesis to create an altered polynucleotide comprising one or more particular substitutions, deletions, or insertions. These and other methods can be used to create derivatives antibodies or variants of anti-EPO (C4), which have different properties, such as more affinity, avidity, stability, and or specificity for human secreted from mammalians (e.g. human, mice, rats, monkeys . . . ) in vitro and in vivo, or reduced in vivo side effects as compared to underivatized antibody.

In another embodiment, the anti-EPO (C4) derivatives can comprise at least one of the above-mentioned CDRs, which may be incorporated into known antibody framework regions, or, in order to increase half-life, stability, safety, and ease of manufacture, conjugated to a carrier, such as: Fc, albumin, transferrin, nanoparticles, lipoproteins, insoluble proteins, such as silk fibroin, biomolecules such as poly(lactic-co-glycolic acid) (PLGA), collagen, keratin, polysaccharides as chitosan, cyclodextrin, hyaluronic acid, heparin, pectin and similar biomolecules.

In certain aspect, variant of the anti-EPO (C4) includes glycosylation variants, wherein the number and/or type of glycosylation sites have been altered compared to the amino acid sequences of a parent polypeptide, such as N-linked glycosylation sites, or substitutions which eliminate an existing N-linked carbohydreate chains wherein one or more N-linked glycosylation sites are eliminated and one or more new N-linked sites are created. Antibody variants can also include cysteine variants, where one or more cysteine residues are eliminated or substituted for another amino acid.

In one aspect, the present invention provides human antibodies that specifically bind to human erythropoietin with a 20 times affinity higher compared to mouse erythropoietin. Such antibodies include antagonizing, or neutralizing antibodies, and no-neutralizing antibodies. Examples are reported in Figure (26).

In certain embodiments, the antibody of the invention binds human erythropoietin with a KD less of 50 nM (ka=3.15E+03(1/Ms), kd=1.57E-04(1/s), KD=4.97E-08(M)).

Antibodies of the invention can be used to assay human erythropoietin levels in biological samples, such as blood and tissues, as diagnostic tools, by classical laboratory methods, as known in the art, such as enzyme-linked immunosorbent assay (ELISA), immunofluorescence, Western blot, radioimmunoassay, The invention provides a method of diagnosis or detection, comprises detecting binding of an anti-human erythropoietin antibody to human EPO expressed on the surface of a cell, or in a membrane preparation, or human EPO in soluble form in human specimens or samples, or solutions.

In a still further aspect, herein described is a pharmaceutical composition comprising (i) the anti-EPO antibody of the present invention or (ii) the polynucleotide encoding said anti-EPO antibody, wherein the composition optionally further comprises a carrier. A pharmaceutical composition may optionally contain other active ingredients. The term “carrier” refers to a vehicle, excipient, diluents, or adjuvant with which the therapeutic or active ingredient is administered. Any carrier and/or excipient suitable for the form of preparation desired for administration is contemplated for use with the strains/wall/postbiotic disclosed herein.

The carrier may take a wide variety of forms depending on the form of preparation desired for administration, e.g. oral or parenteral, including intravenous. In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents, and the like in the case of oral solid preparations such as, for example, powders, hard and soft capsules and tablets, with the solid oral preparations being preferred over the liquid preparations.

In a preferred aspect, the composition of the invention is for oral, or parenteral, topical, rectal, intravenous, subcutaneous, intramuscular, intranasal, intravaginal, intravitreally through the oral mucosa, the lung mucosa, or for transocular administration.

The compositions include compositions suitable for parenteral, including subcutaneous, intramuscular, and intravenous, pulmonary, nasal, rectal, topical or oral administration. Suitable route of administration in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy. The preferred compositions include compositions suitable for oral, parenteral, topical, subcutaneous, or pulmonary, in the form of nasal or buccal inhalation, administration. The compositions may be prepared by any of the methods well-known in the art of pharmacy.

In a preferred aspect said pharmaceutical composition is administered incorporated into liposomes, microvescicles, bound to molecular carriers or combined with molecules selected from the group consisting of molecules that allow the temporary opening of the blood-brain barrier, anti-inflammatory molecules, monoclonal antibodies, drugs with immunosuppressive activity, nanoparticles, gold nanoparticles, mucoadhesive nanoparticles based on poly(lactic-co-glycolic acid) (PLGA) and oligomeric chitosan (OCS) conjugated with monoclonal antibody and or chemotherapy compounds

In another aspect, the invention provides for an antibody comprising (a) a variable domain of a light chain (VL) having the amino acid sequence of SEQ ID NO:6; and (b) a variable domain of a heavy chain (VH) having the amino acid sequence of SEQ ID NO:14, the composition or the immunoconjugate as herein described, for use as a medicament.

The anti-EPO monoclonal antibody (also referred to as “C4”), described and claimed in the present invention, has surprisingly been shown to be able to induce apoptosis in cancer stem cells, to inhibit their growth and tumor angiogenesis.

In a further aspect, the invention provides for an antibody comprising (a) a variable domain of a light chain (VL) having the amino acid sequence of SEQ ID NO:6; and (b) a variable domain of a heavy chain (VH) having the amino acid sequence of SEQ ID NO:14, the composition or the immunoconjugate as herein described, for use in the treatment of a tumor, cancer, or cell proliferative disorder, and/or for inhibiting angiogenesis or vascular permeability, treating an autoimmune and non-autoimmune based chronic inflammatory diseases, in the treatment of patients undergoing organ or tissue transplant, in the treatment of haemophilic arthropathy, and neurodegenerative diseases and neurological diseases in which abnormal or excessive activation of the autoimmune system has a pathogenic role or in which neuro inflammation plays a role in pathogenesis, for example: multiple sclerosis, Parkinson's disease, Alzheimer's disease, frontotemporal dementia, dementia with Lewy bodies, autoimmune disease with neurologic involvement, Amyotrophic Lateral Sclerosis, and Neuromuscular Diseases, in ophthalmic pathologies, such as neovascular age related (NVAMD), macular degeneration, retinal vein occlusion (RVO), metabolic syndromes, diabetes, and neuropathic pain disorders,

In a preferred aspect said tumor, cancer, or cell proliferative disorder is chosen from the group consisting of cerebral astrocytoma, cerebellar astrocytoma, astrocytoma of the pineal gland, oligodendroglioma, pituitary adenoma, craniopharyngioma, sarcoma, glioblastoma grade II fibrillary astrocytoma, protoplasmic, grade III gemistocytic, anaplastic astrocytoma, including gliomatosis cerebri, pituitary adenoma, ependymoma, medulloblastoma, neural ectoderm tumor, neuroblastoma, hypothalamic glioma, breast cancer, lung cancer, colon cancer, cervical cancer, endometrial cancer, uterine cancer, ovarian cancer, esophageal cancer, basal cell carcinoma, cholangiocarcinoma, cancer of the spleen, osteosarcoma, intraocular melanoma, retinoblastoma, stomach cancer, heart cancer, liver cancer, hypopharyngeal cancer, laryngeal cancer, cancer of the oral cavity, nasal and paranasal cancer, cancer of the salivary glands, nasopharyngeal cancer, throat cancer, thyroid cancer, pancreatic cancer, kidney cancer, prostate cancer, bladder cancer, rectal cancer, testicular cancer, renal cell cancer melanoma, sarcoma, mesothelioma, pheochromocytoma, and hematological cancers. More preferably said tumor, cancer, or cell proliferative disorder is chosen said cancer is selected from the group consisting of: glioblastoma, anaplastic astrocytoma, colon cancer, prostate cancer, lung cancer, breast cancer, cancer, endometrial cancer, uterine cancer, ovarian cancer and said hematological cancer is leukemia.

As will be further described in the Examples section, surprisingly, the anti-EPO antibody was able to induce apoptosis in human glioblastoma stem cells, in tumor endothelial cells, in cancer cells derived from anaplastic astrocytomas, colon cancer, prostate cancer, breast cancer, leukemia, ovarian cancer, and lung cancer. Interestingly the treatment of anti-EPO (C4) mAB did not affect the viability of health human astrocytes. In parallel the effect of anti-EPO (C4) was studied on cell angiogenesis, migration and apoptosis. Results demonstrated that the treatment with anti-EPO (C4) is able to inhibit tumor endothelial angiogenesis, as well as cancer cell migration in an experimental model of glioblastoma endothelial cells and colon cancer cells. Moreover, the combined treatment carried out on cancer stem cells with the anti-EPO antibody and FTY720 and/or temozolomide showed a superior effect in terms of induction of apoptosis and of blocking tumor growth, compared to the effect measured by anti-EPO, FTY720 and temozolomide tested individually. The results obtained with the negative functional modulators of EPO, claimed herein and reported in the examples that follow, show the surprising effectiveness of anti-EPO (C4) mAB particularly because they were obtained in an in vitro model characterized by a strong resistance to apoptotic stimuli.

Interestingly, potent anti-cancer effects were evaluated when the cancer cell models were treated with anti-EPO (C4) in combination with anti-EPHB4, a monoclonal antibody raised against amino acids 201-400 mapping within an extracellular domain of EphB4 of human origin.

Importantly, antibody C4 has been seen to have: i) a high affinity for human EPO; ii) in in xenograft animal model no renal and liver toxicity; no influence on hematopoiesis; iii) good stability in the bloodstream; iv) efficient uptake in xenograft tumor tissues; vi) activity in modulating gene expression on tumoral cells, where the in vivo treatment induced inhibition cancer stem cell proliferation, increase apoptosis and inflammation. Within the scope to evaluate the effects of anti-EPO treatment on chronic inflammatory conditions, it was decided to perform experiments on microglial cells, as a neuro-inflammatory disease model. These cells are principally involved in the maintenance and amplification of the neuroinflammatory state, through the production of pro-inflammatory molecules such as cytokines and chemokines. However, prolonged and uncontrolled microglial activation is harmful for neurons and thus the inhibition of the prolonged neuroinflammatory state constitutes today a target of strategies to limit neuronal damage. To test this hypothesis, N9 microglia cells (a cell line of immortalized murine microglia), cultured in the presence of lipopolysaccharide (LPS), as potent inflammatory stimulus, were used and subjected to treatment with monoclonal anti-EPO (C4) antibody+FTY720 and anti-EPHB4. The effects on the proliferation and migration of activated microglia were assessed.

Results showed that treatment with LPS induced a potent proliferation and migration stimulus. Treatment with anti-EPO (C4) antibody, according to the invention, in association with or without FTY720 and its analogues, as shown in the examples that follow, inhibits the proliferation, migration and survival of activated microglia. These effects were enhanced by the association of two principles. Surprisingly, the treatment with anti-EPO (C4) in combination with anti-EPHB4 did not affected microglia viability, but interestingly, induced a decrease in cell proliferation.

In the context of the effects on inflammatory-like diseases such as haemophillic arthropathy, endothelial cells were isolated from the synovium of patients affected by haemophilia (S-ECs).

Hemophilic arthropathy (HA) is a frequent, significant complication of hemophilia, that may lead to poor quality of life. In the complexities of HA pathophysiology, different studies showed that aside from bleeding, an intense neovascularisation of the synovial membrane plays a crucial role in promoting the cycle of recurrent hemarthroses and inflammation. Indeed, hemophilic synovial tissue is predisposed toward an exuberant neoangiogenesis, characterized by aberrant endothelial proliferation, and inflammatory cell invasion, where S-ECs assumed a pro-tumoral angiogenic morphology. For the first time, we demonstrated that the treatment with anti-EPO (C4) did not affect S-EC viability, but, surprisingly, had an effect on cell proliferation, and angiogenesis with reduced stabilization and vessel maturation (tumor-like) at the synovial level.

The abnormal proliferation and altered maturation of vessels associated with an inflammatory state also manifests itself in other coagulation disorders comprising hemophilia A and B, von Willebrand's disease and angiodysplasia associated therewith. Chronic inflammation is common to this phenotype, to that of cancer stem cells/tumor tissues and other inflammatory diseases such as rheumatoid arthritis. In this sense, the data obtained (inserted in the examples below) show that treatment with anti-EPO (C4) mAB is able to inhibit pathological synovial endothelial proliferation, having pro-apoptotic, and also abolishing the initial inflammatory stimulus. The negative modulators of EPO according to the present invention can therefore be used for direct intra-articular treatment in the form of a gel or suspension, in association or not with “coagulation factors and their derivatives” and FTY720 if necessary and/or negative modulators of the sphingosine-1-phosphate pathway and/or inhibitors of EPO and receptors, and/or inhibitors of VEGF and receptors. Alternatively, it is possible to use the negative modulators of EPO for topical or systemic application, as well as in the form of microparticles, microvescicles, liposomes etc. Alternatively, the administration may be achieved through the use of all those technologies currently related to gene therapy, or the use of vectors for the introduction of nucleic acids into cells of the patient. Such administration can be effective at a systemic level, then by infusion, or at a local level, with the administration of vectors directly into the site of the lesion, tumor, synovial, cerebral etc.

The composition of the present invention can be a pharmaceutical composition for use in the treatment of malignancies, in the therapy of autoimmune and non-autoimmune based chronic inflammatory diseases, in the treatment of patients undergoing an organ or tissue transplant, in the treatment of hemophilic arthropathy and in the treatment of neurological disorders in which neuroinflammation has a role in the pathogenesis, that comprises a negative functional modulator of EPO according to the present invention in therapeutically effective concentrations and pharmaceutically acceptable excipients. Preferably, said composition further comprises a therapeutically effective amount of one or more natural or synthetic molecules that act on the receptors of S1 P, and/or on the metabolism of S1 P directly or indirectly, and/or anticancer cytotoxic molecules and/or antiviral and/or anti-angiogenic, and/or a therapeutically effective amount of one or more natural or synthetic molecules that act on EPO receptors (EPOR, EPHB4, CSF2RB), directly or indirectly, also in association with EPO mimetics.

Even more preferably, said molecule which acts on the receptors of S1 P, and/or on the metabolism of S1 P directly or indirectly, is FTY720 or its analogues. Preferably, said anticancer cytotoxic molecule and/or antiviral and/or anti-angiogenic is selected in the group comprising: paclitaxel, taxol, cycloheximide, carboplatin, chlorambucil, cisplatin, colchicine, cyclophosphamide, daunorubicin, dactinomycin, diethylstilbestrol, doxorubicin, etoposide, 5-fluorouracil, floxuridine, melphalan, methotrexate, mitomycin, 6-mercaptopurine, teniposide, 6-thioguanine, vincristine and/or vinblastine, fotemustine, carmustine, irinotecan systemically or by carmustine adsorbed biopolymer wafers for locoregional therapy, temozolomide, tamoxifen, valganciclovir, ganciclovir, acyclovir, anti-VEGF, anti-VEGFR, anti-HER2/neu, anti-EGFR, gefitinib, bevacizumab, ranibizumab, vatalanib, Cediranib, Sorafenib, Sunitinib, Motesanib, Axitinib. Preferably, said molecules that act on EPO receptors are polyclonal or monoclonal antibodies directed against EPOR, EPHB4, CSF2RB.

Furthermore, it was surprisingly it was seen that the anti-EPO antibody can be used in the treatment of patients which are resistant or intolerant to previous treatment with at least one antitumor agent or wherein the treatment with an antitumor agent should be avoided. The treatment with anti-EPO (C4) on cell resistant to chemotherapeutic compounds such as temozolomide (TMZ) and carboplatin (CPT), surprisingly, demonstrated to sensitizes resistant cancer stem cells (Example 17, FIG. 24), In a further aspect, the invention describes a method of treatment comprising the step of administering anti-EPO antibody of the present invention, said composition or said immunoconjugate to a subject in need thereof.

Taken together, the experimental results obtained which will described in the Examples section appear to allow the exploration of strategies to simultaneously block EPO and S1 P function to target tumor growth and angiogenesis may be warranted, especially in aggressive solid and hematological tumors. In parallel, the results obtained on the inhibition of the pro-inflammatory action of microglial cells stimulated with lypopolisacharide by the anti-EPO monoclonal antibody, paves the way for new treatments of autoimmune and non-autoimmune based chronic inflammatory diseases, in the treatment of patients undergoing organ or tissue transplant, in the treatment of haemophilic arthropathy, and in neurological diseases in which neuro-inflammation plays a role in pathogenesis, for example: multiple sclerosis, Parkinson's disease, Alzheimer's disease, frontotemporal dementia, dementia with Lewy bodies, autoimmune disease with neurologic involvement, Amyotrophic Lateral Sclerosis, Neuromuscular Diseases, and in pathology in which aberrant angiogenesis play a pivotal role in pathogenesis, such as ophthalmic pathologies such as neovascular age related (NVAMD), macular degeneration, retinal vein occlusion (RVO), metabolic syndromes, diabetes, and neuropathic pain disorders

In a still further aspect, the present invention describes a diagnostic method for measuring the amount of EPO protein in a sample previously obtained from a human or animal subject, comprising the step of using the C4 antibody of the invention.

Preferably, said sample is chosen from the group consisting of cell, tissue, blood, saliva and cerebrospinal fluid and said diagnostic method is carried out by one or more of: ELISA assay, Western blot analysis, RealTime PCR or PCR, functional angiogenesis assays and drug screening platform alone or in combination.

Still more preferably, the diagnostic method of the invention is carried out with the further step of using an EPO receptor. The use of the EPO receptor can be before, after or concomitant with the use of the antibody of the invention, and said EPO receptor can be chosen from the group consisting of EPOR, EPHB4, CSFR2B, tissue protection factor (TPR, EPOR/CD131 heterodimer).

In a still further aspect, herein described is a pharmaceutical kit comprising the antibody of the invention and one or more compounds chosen from the group consisting of a negative functional modulator of S1 P signaling or of an anti-EPO receptor selected from the group comprising EPOR, EPHB4, CSFR2B, tissue protection factor (TPR, EPOR/CD131 heterodimer), and/or EPO mimetics, for simultaneous, separate or sequential administration.

In a still further aspect, the invention relates to a hybridoma which is deposited under deposit Accession No. DSM ACC 3370 by the International Deposit Authority DSMZ, Braunschweig, Germany.

In a further aspect the invention describes an anti-Erythropoietin (EPO) monoclonal antibody produced by the hybridoma deposited under deposit Accession No. DSM ACC 3370 by the International Deposit Authority DSMZ, Braunschweig, Germany.

The anti-Erythropoietin (EPO) monoclonal antibody produced by the hybridoma deposited under deposit Accession No. DSM ACC 3370, comprises a variable domain of a light chain (VL) having the amino acid sequence of SEQ ID NO:6; and b. a variable domain of a heavy chain (VH) having the amino acid sequence of SEQ ID NO:14.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention.

### Example 1: Anti-EPO Antibody Generation

The techniques and procedures described or referenced herein are generally well understood and commonly employed using conventional methodology by those skilled in the art, such as, for example, the widely utilized hybridoma methodologies.

An exemplary protocol used to generate the anti-EPO (herein also named “C4”) monoclonal antibody using the hybridoma method is described as follows.

N=5 mice were immunized to elicit lymphocytes produced and capable of producing antibodies that specifically bounded to the protein used for immunization (human EPO, PeproTech #100-6). The following immunization protocol was used:
Day 1: 1st-immunization (CFA adjuvant);
Day 14: 2nd-immunization (IFA adjuvant);
Day 28: 3rd-immunization (IFA adjuvant);
Day 35: ELISA titer test;
Day 42: 4th-immunization (IFA adjuvant);
Day 49: ELISA titer test;
Day 51: Fusion if final boost at D42;
Day 56: 5th-immunization (IFA adjuvant);
Day 63: ELISA titer test; Day 65: Fusion if final boost at D56;
Day 70: 6th-immunization (IFA adjuvant); Day 77: ELISA titer test;
Day 79: Fusion if final boost at D70.

The Antibodies were raised in animals by multiple subcutaneous (sc) or intraperitoneal (ip) injections. Serum from immunized animals were assayed for anti-EPO antibodies by ELISA test. Lymphocytes from animals producing anti-EPO antibodies were isolated and then fused with myeloma cells using a suitable fusing agent, such as polyethylene glycol, to form a hybridoma cell. After fusion n=5 clones were selected after fusion. The hybridoma cells, thus prepared, were seeded and grown in the following conditions: RPMI 1640+10% FBS+1% Gln+1% Pen/Strep, at 37° C., 95% relative humidity, 5% C02. Hybridomas were splitted 1:5 approximately every 3 days. The binding specificity of monoclonal antibodies produced by hybridoma cells were determined by enzyme-linked immunoadsorbent assay (ELISA) and by neutralizing activity treating cancer cells with the clones (see the example reported below).

### Neutralizing Anti-EPO (C4) Bioassay

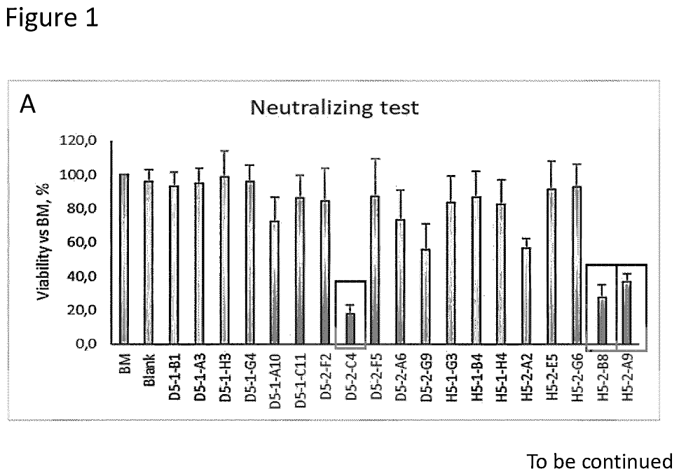

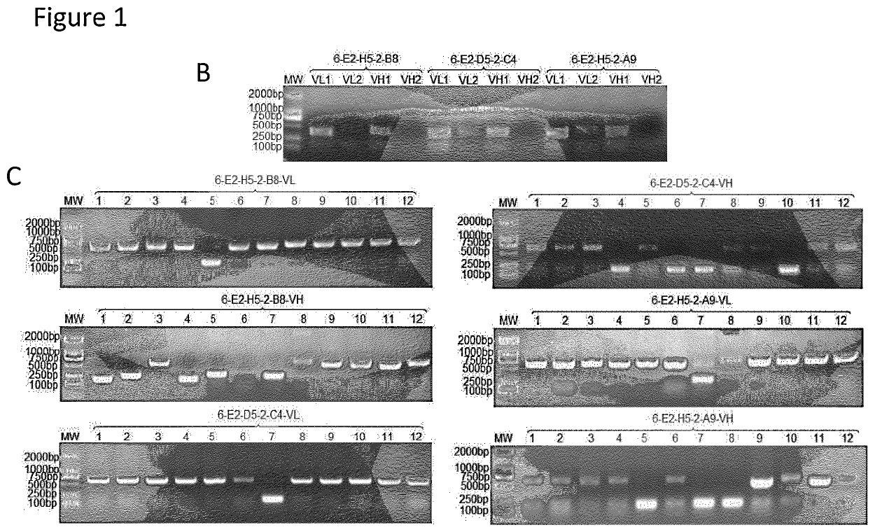

The cell-based assay was used to measure the capacity of each clone to neutralize EPO signaling. In briefly, cells (5×10³/well) were seeded and cultured in 96-well plate for 24 h in Basal Medium (BM). Then, culture media were replaced with fresh media containing the specific treatments, or in BM as a control condition (CTR). After 96 h cell viability was measured by 3-(4,5-Dimethylthiazol-2-yl)-2,5-Diphenyl-tetrazoliumBromide (MTT) assay, as a function of redox potential. Optical density values (O.D.) were recorded, and neutralizing activity of each clone was expressed as percentage of viability of cells treated with clones respect to control condition, BM (FIG. 1A).The best n=3 final clones were selected and specific monoclonal antibodies were separated and purified in PBS at PH7.4 sterilized by membrane filtration (produced in culture supernatant and purified by Protein A/G). Furthermore, anti-EPO monoclonal antibodies were sequenced by the following protocol: total RNA was extracted separately from several batches of cultured hybridoma cells, cDNA were then synthesized by reverse transcription using oligo-dT primers, and VH and VL were finally amplified by PCR. VH and VL fragments, respectively amplified by IgG degenerate primers and Kappa-specific primers, are represented by gel electrophoresis (FIG. 1B), confirming that isotypes were IgG Kappa.

The PCR products were then sub-cloned into a standard vector, followed by bacteria transformation, then colony picking and validation by PCR (FIG. 1C), and finally sequencing of 8-11 positive clones for each VH and VL. Surprisingly, all clones had identical VL and VH, so 6-E2-H5-2-A9, 6-E2-H5-2-B8 and 6-E2-D5-2-C4 have the same sequences and are the same clone, The anti-EPO antibody of the present invention is “C4” (6-E2-D5-2-C4).

### Example 2: Cell Lines and Treatments Used

The following cellular models were used:

A. Primary glioblastoma cancer stem cells (GCSc). Anti-EPO treatments were performed on glioblastoma cancer stem cells (GSCs) isolated from tumor biopsy of a coohort of glioblastoma patients, in order to demonstrate a specific anti-tumor efficacy, achieved by the inhibition of proliferation and survival and the induction of apoptosis (see below).

B. Primary glioblastoma endothelial cells (GECs). Anti-EPO treatments were performed on glioblastoma endothelial cells (GECs) isolated from the vascular compartment of glioblastoma biopsies, in order to demonstrate a specific anti-tumor efficacy, both at cellular and functional point of view, through the inhibition of angiogenesis and proliferation (see below).

C. Glioblastoma cancer stem cell commercial line (Glioblastoma stem cell line, GBM-CSCs). Anti-EPO treatments were performed on GBM-CSCs, a commercial glioblastoma cancer cell line by CelProgen. As reported in the datasheet, GBM-CSCs have been isolated from human brain cancer tissue. GBM-CSCs were maintained in Celprogen's human glioblastoma cancer stem cell (GBM) complete growth medium and subcultured every 24 to 48 hours on a specific extra-cellular matrix.

D. Primary anaplastic astrocytoma cancer stem cells isolated from tumor biopsy of a cohort of patients affected by high grade glioma, in particular, anaplastic astrocytoma (WHO, grade III);

E. Colon cancer cell line (DLD1); Epo has been shown to have a serious negative effect in promoting the neoplastic process of colon cancer by enhancing carcinogenesis by increasing EpoR expression;

F. Prostate cancer cell lines (PC-3 and LNCAP); human prostate cancer cells used in cancer research and drug development. Human hepatocellular receptors (Ephs) producing erythropoietin have been reported to be overexpressed and associated with poor prognosis and reduced survival in prostate cancer patients, and are considered predictive markers of aggressive prostate cancer behavior. Furthermore, it has recently been shown that in LNCAP, the simultaneous overexpression of Epo and EpoR in resistant prostate cancer plays an important role in progression and is responsible for the development of a neuroendocrine phenotype;

G. Breast cancer cell line (MCF-7); breast cancer cells. This cell line has been shown to be reactive to human erythropoietin (rHuEPO) treatment in terms of increased cell proliferation;

H. Myelogenous leukemia cell line (K562); chronic myeloid leukemia cells. Circulating dipo levels are reliably higher in myelodysplastic syndromes than in healthy people, with a negative predictive role;

I. Ovarian cancer cell line (A2780); ovarian cancer cells used in toxicity testing, drug screening and genetic cancer studies. Previous studies have shown that A2780 express EPOR and that treatment with Epo resulted in increased resistance to chemotherapy.

J. Lung carcinoma cell line (A549): human lung cancer cells used for drug efficacy screening, biochemical mechanisms and alveolar differentiation;

K. Neuroblastoma cell line (SHSY-5Y) line is a cell line isolated from a bone marrow biopsy taken from a patient with neuroblastoma. SHSY cells are often used as in vitro models of oncological pathology and neuronal differentiation.

L. Human healthy Astrocytes is a commercial cell line of human healthy astrocytes

M. Synovial endothelial cells (S-ECs): endothelial cells isolated from biopsies of synovium of patients affected by haemophilia.

N. Human ovarian Cell (HOC) cultured in stem cell condition. In particular, HOC84 stem cells were obtained by digestion and cell isolation form a patient-derived xenograft (PDX) model from a high grade serous epithelial ovarian cancer, obtained by serial passages in nude mice.

The following treatments were used:
1. anti-human EPO (C4) monoclonal antibody: anti-human EPO mAB, obtained by hybrodoma technique, which recognize the aminoacid sequence of human EPO (aa 28-193);
2. Treatment with Temozolomide (TMZ), used as standard treatment in patients with glioblastoma
3. Treatment with Fingolimod (FTY720), used as a functional S1 P antagonist;
4. Anti-human EPHB4: a mouse monoclonal antibody raised against amino acids 201-400 mapping within an extracellular domain of EphB4 of human origin (Santacruz Biotechnology, Inc, EphB4 (H-10): sc-365510.
5. lipopolysaccharide (LPS), at a concentration of 3 μg/ml.
6. Treatment with Carboplatin (CPT), a therapeutic treatment in patients with ovarian cancer. Example 3: Cell viability on human cancer cells.

Cell viability was assessed by 3-(4,5-Dimethylthiazol-2-yl)-2,5-Diphenyl-tetrazoliumBromide (MTT) assay, as a function of redox potential (FIGS. 2-7). Cells (5×10³/well) were seeded and cultured in 96-well plate for 24 h in Basal Medium (BM). Then, culture media were replaced with fresh media containing the specific treatments, or in BM as a control condition (CTR). The following treatments were administered:
Control (at time 0, replacement of the culture medium);
anti-EPO (C4) (at time 0, replacing the culture medium with culture medium containing 6 μg/ml of anti-EPO (C4), monoclonal antibody against AA 28-193 of human EPO);
TMZ (the culture medium was replaced at time 0 with fresh culture medium, containing TMZ at 100 μM);
FTY720 (the culture medium was replaced at time 0 with fresh culture medium, containing FTY720 at 100 nM);
CPT (the culture medium was replaced at time 0 with fresh culture medium, containing CPT at 40 μg/mL);
TMZ+FTY720
anti-EPO (C4)+TMZ+FTY720
anti-EPHB4 (at time 0, replacing the culture medium with culture medium containing 6 μg/ml of anti-EPHB4);
anti-EPO (C4)+anti-EPHB4
anti-EPO (C4)+anti-EPHB4+TMZ and/or FTY720
anti-EPO (C4)+CPT

Test was performed in triplicate after 96 h of treatment, by replacing culture media with 100 μL of fresh media added with 10 μL of MTT 5 mg/ml in D-PBS. After 4 h of incubation, media were removed and cells were lysed with 100 μL of 2-propanol/formic acid (95:5, by vol) for 10 min. Then, absorbance was read at 570 nm in microplate reader.

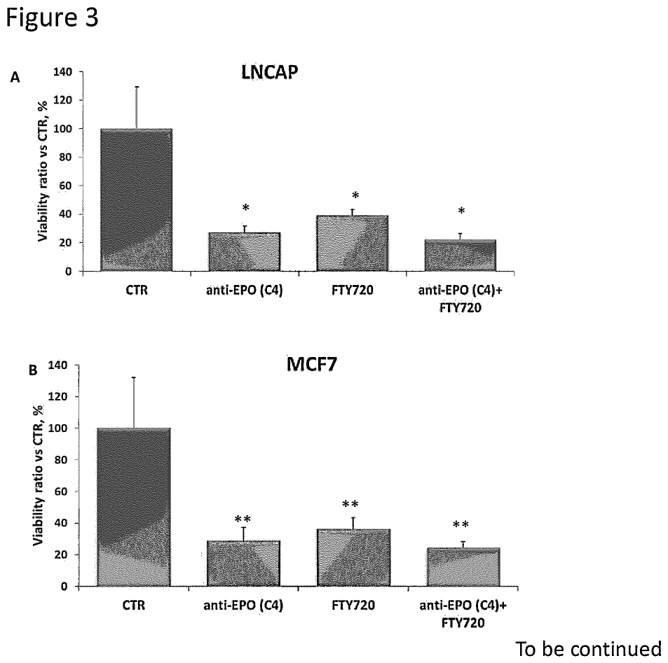

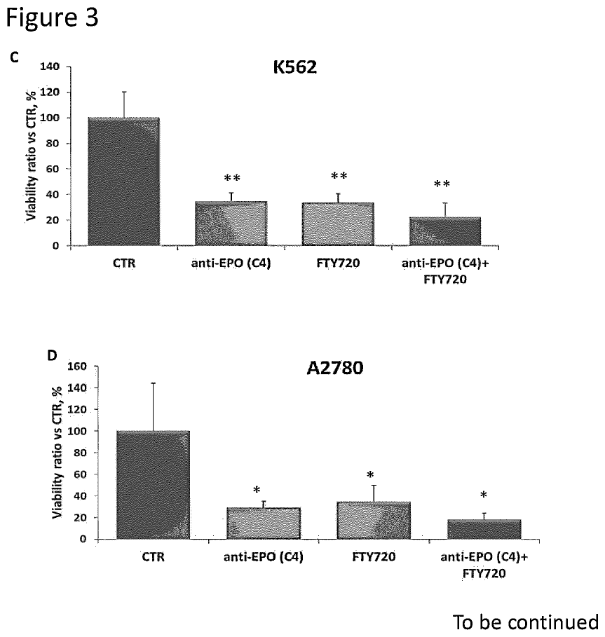

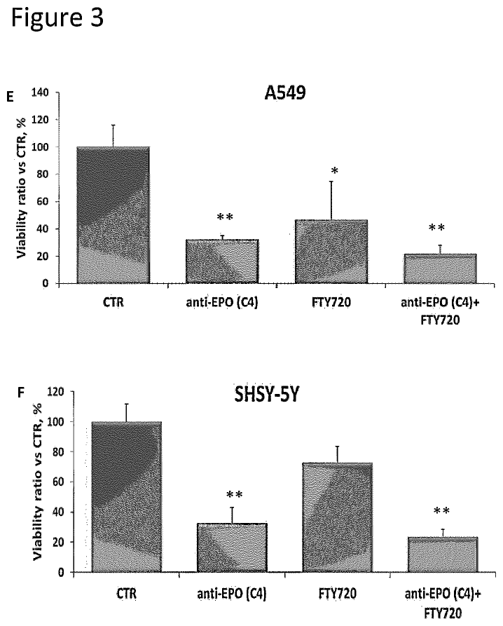

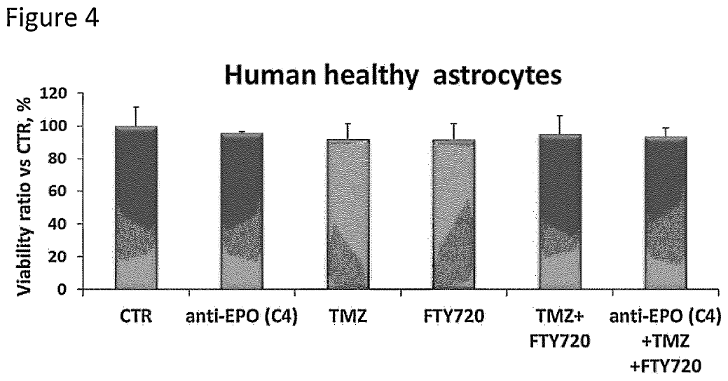

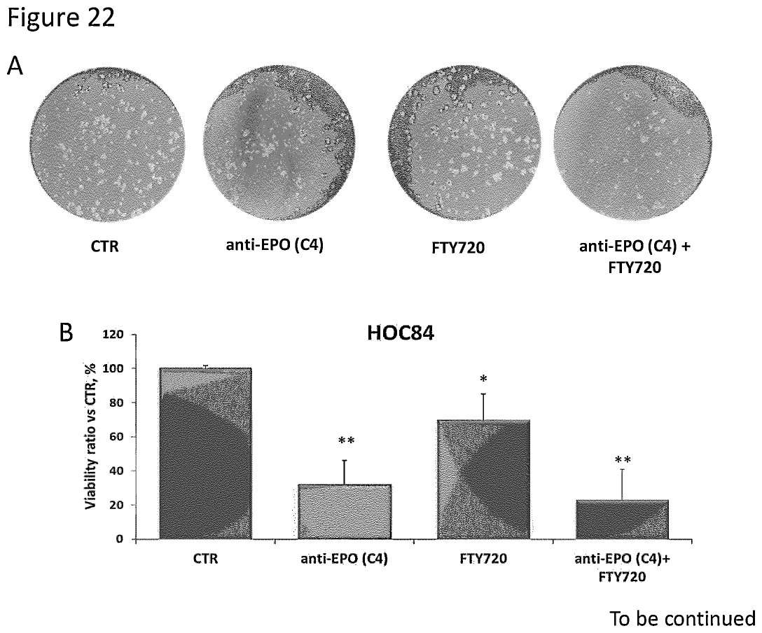

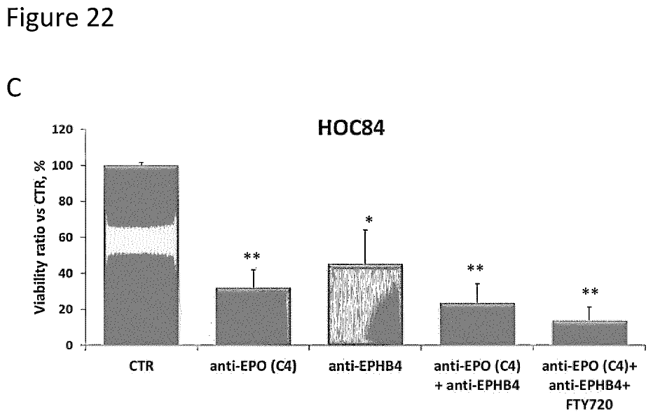

The analysis of cell viability shows that, at 96 hours after treatment with anti-EPO (C4), about 73% for primary GSCs (FIG. 2A), 70% for primary GECs (FIG. 2B), 79% for GBM-CSC cell line (FIG. 2C) and 66% for anaplastic astrocytoma cells (FIG. 2D) initially seeded were dead (FIG. 2). Interestingly, the co-administration of anti-EPO mABs with TMZ and FTY720, increased the mortality up to 79% for primary GSCs, 75% for primary GECs, 87% for GBM-CSCs, and 72% for anaplastic astrocytoma (FIG. 2D). Interestingly, the analysis of cell viability shows that, also for the other cancer cell models used herein, the treatment with anti-EPO (C4) resulted in viability decrease: 73% for DLD1 (FIG. 2E), 71% for PC-3 (FIG. 2F), 74% for LNCAP (FIG. 3A), 71% for MCF-7 (FIG. 3B), 62% for K562 (FIG. 3C), 63% for A2780 (FIG. 3D), 63% for A549 (FIG. 3E), 62% for SHSY-5Y (FIG. 3F), and 68% for HOC84 (FIG. 22B) of the cells initially plated were dead (FIGS. 2, 3 and 22). Interestingly, the co-administration of anti-EPOmABs with FTY720 and/or TMZ, inhibited cell growth by an average of 75% in all cancer models tested. Importantly, the treatment with anti-EPO (C4) did not affected human healthy astrocyte viability (FIG. 4C).

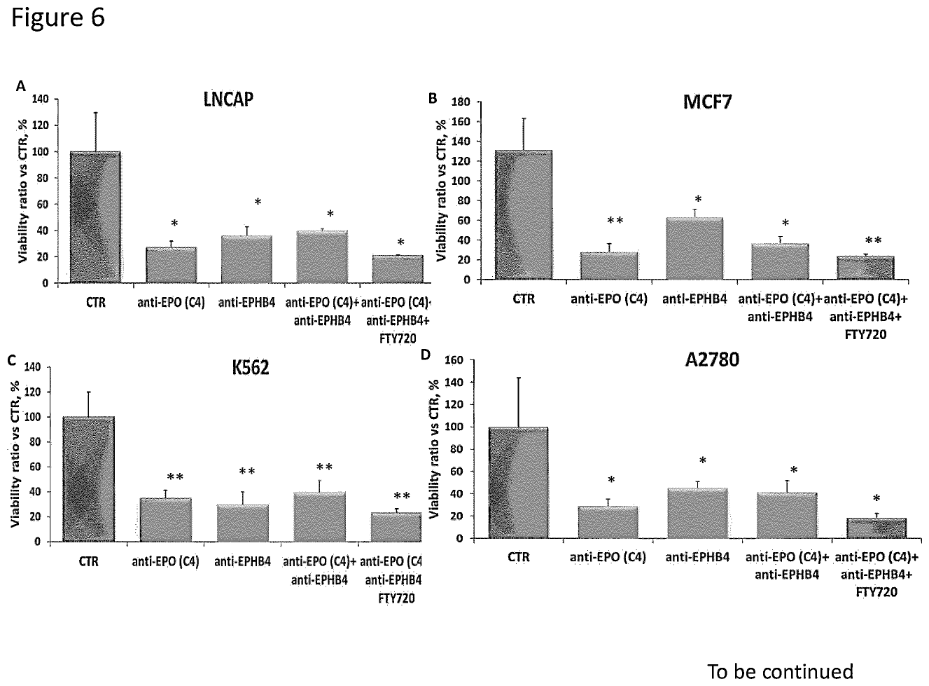

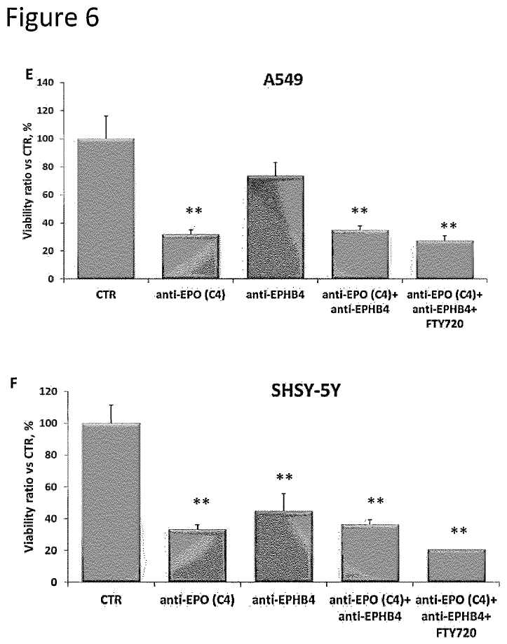

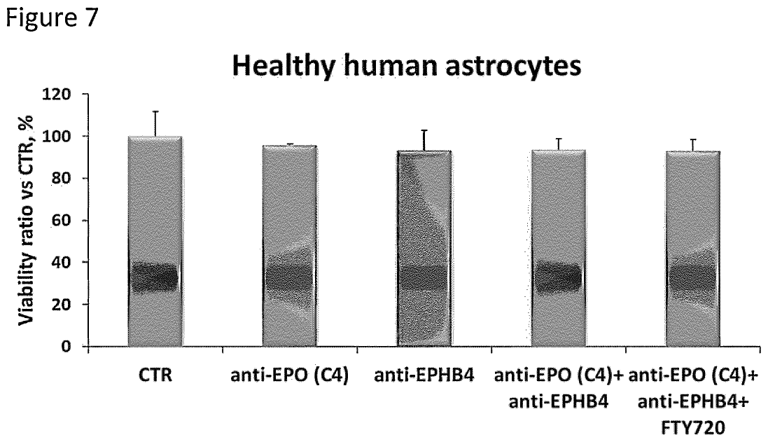

Furthermore, the analysis of cell viability shows that, at 96 hours after treatment with anti-EPO (C4) combined with anti-EPHB4 and/or FTY70 and/or TMZ, only about 27% of primary GSCs (FIG. 5A), 20% for primary GECs (FIG. 5B), 19% for GBM-CSC cell line (FIG. 5C) and 14% for anaplastic astrocytoma cells (FIG. 5D) initially seeded were alive (FIG. 5). Interestingly, the co-administration of anti-EPO (C4) with anti-EPHB4 and/or TMZ and FTY720, decrease the viability also in the other cancer model indications. In detail following the combined treatment only 17% for DLD1 (FIG. 5E), 15% for PC-3 (FIG. 5F), 21% for LNCAP (FIG. 6A), 25% for MCF-7 (FIG. 6B), 23% for K562 (FIG. 6C), 18% for A2780 (FIG. 6D), 27% for A549 (FIG. 6E), 21% for SHSY-5Y (FIG. 6F), and 14% for HOC84 (FIG. 22C) of the cells initially plated were still alive (FIGS. 5,6 and 22). Interestingly, the co-administration of anti-EPO (C4) with anti-EPHB4 and with/or FTY720 and/or TMZ, did not affect human healthy astrocyte viability (FIG. 7B).

Data are expressed as the mean±standard deviation of at least 3 experiments in triplicate. * P<0.05; **P<0.01; ***P<0.001 versus CTR for all treatments tested.

### Example 4: Functional Test of New Vessel Formation: Tube Formation Assay

GBM-ECs (1×10⁴) were plated on 10 μL of Matrigel in BM, as a Control Condition (CTR, FIG. 8A) or in media containing the anti-EPO (C4) (FIG. 8B), TMZ (FIG. 8C), FTY720 (FIG. 8D), and anti-EPO (C4)+TMZ+FTY720 (FIG. 8E) treatments, as reported above. GBM-ECs were incubated at 37° C., 5% C02, 5% 02. After 48 h, tube-like structure formation was evaluated by phase-contrast microscopy.

It was noted that the anti-EPO (C4) mAB was able to block the formation of tubular-like structures (FIG. 8B), while TMZ (FIG. 8C) and FTY (FIG. 8D) did not show such a significant effect. The total length of formed tubes in the assay, measured using the Angiogenesis Analyzer plugin in ImageJ, show a 70% decrease after administration of the anti-EPO (C4) treatment (FIG. 8F), which increase up to 75% when co-administered with TMZ and FTY720 (FIG. 8F), indicating a more potent effect. Data are expressed as the mean±standard deviation of at least 3 experiments in triplicate. *P<0.05; **P<0.01 versus CTR for all treatments tested.

### Example 5: Migration Assay on Primary GECs

GBM-ECs (2×10⁴) were plated into the compartments of the insert in BM and incubated at 37° C., 5% CO₂ and 5% O₂. After 24 h, the insert was removed and the cells were cultured for another 48 h in Control Condition (CTR, FIG. 9A) or in the presence of the anti-EPO (C4) treatment, alone (FIG. 9B), with TMZ (FIG. 9C), FTY720 (FIG. 9D) or in co-administration (FIG. 9E) as reported above. After 48 h, the cells were stained with Calcein-AM at 1 μg/mL (Invitrogen) and photographs were acquired with an inverted Leica DMI6000B microscope (Leica Microsystems) equipped for time-Lapse video-microscopy in five random fields (FIG. 9F). Cells migrated into the gap were counted using ImageJ/Analyse Particles (FIG. 9F).

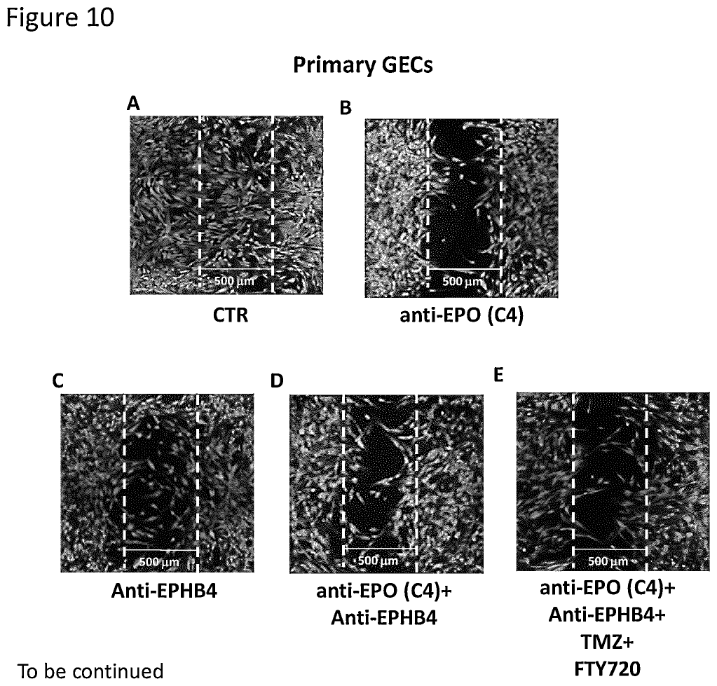

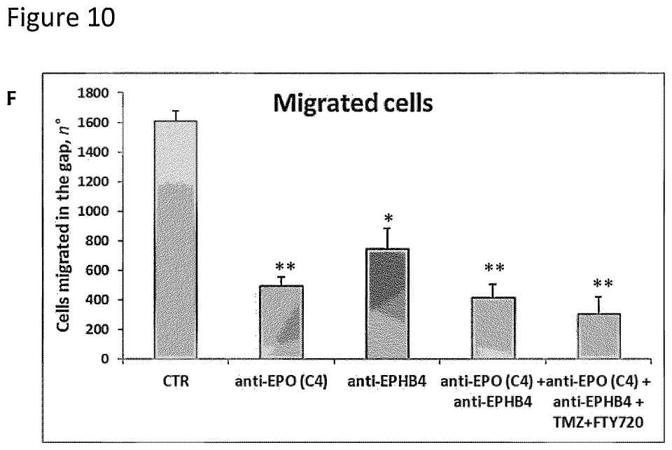

Following treatments with anti-EPO (C4) mAB, a 70% decrease of migration was recorded. The effect was more potent when anti-EPO (C4) mAB was administered with TMZ and FTY720 (78%). In addition, GECs were treated in CTR condition (FIG. 10A), with anti-EPO (C4) alone (FIG. 10B), anti-EPHB4 (FIG. 10C), anti-EPO (C4)+anti-EPHB4 (FIG. 10D) or in combination with TMZ and FTY720 (FIG. 10E). Results showed that the combined treatment was more potent, increasing the migration inhibitory effect up to 81% (FIG. 10F). Data are expressed as the mean±standard deviation of at least 3 experiments in triplicate. *P<0.05; **P<0.01 versus CTR for all treatments tested.

### Example 6: Migration Assay on Primary DLD1

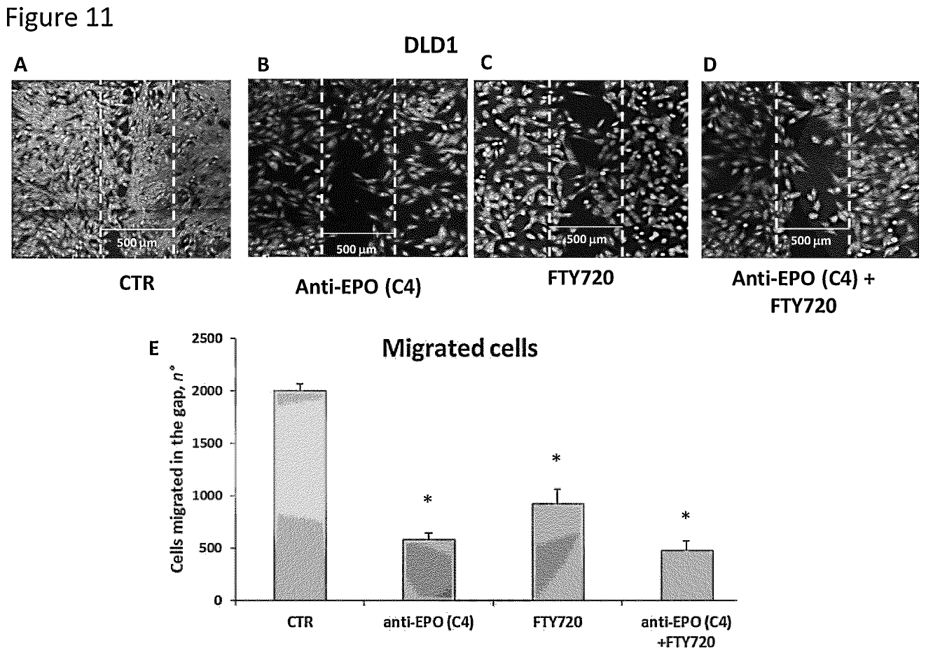

DLD1 (2×10⁴) were plated into the compartments of the insert in BM and incubated at 37° C., 5% CO₂. After 24 h, the insert was removed and the cells were cultured for another 48 h in Control Condition (CTR, FIG. 11A) or in the presence of the anti-EPO (C4) (FIG. 11B), FTY720 alone (FIG. 11C) or in combination (FIG. 11D), as reported above. After 48 h, the cells were stained with Calcein-AM at 1p g/mL (Invitrogen) and photographs were acquired with an inverted Leica DMI6000B microscope (Leica Microsystems) equipped for time-Lapse video-microscopy in five random fields (FIG. 11). Cells migrated into the gap were counted using ImageJ/Analyse Particles (FIG. 11E).

Following treatments with anti-EPO (C4) mAB, a 70% decrease of migration was recorded. The effect was more potent when anti-EPO (C4) mAB was administered with FTY720 (76%).

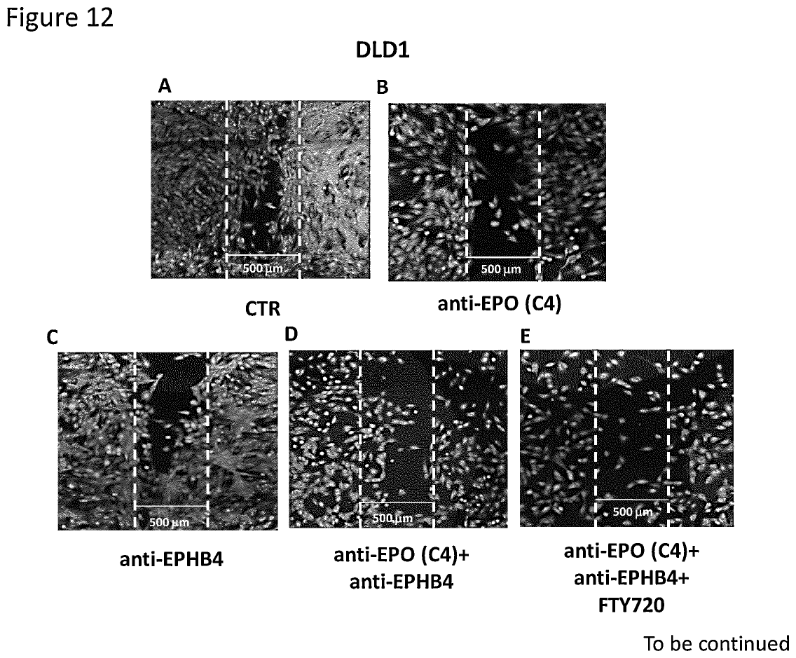

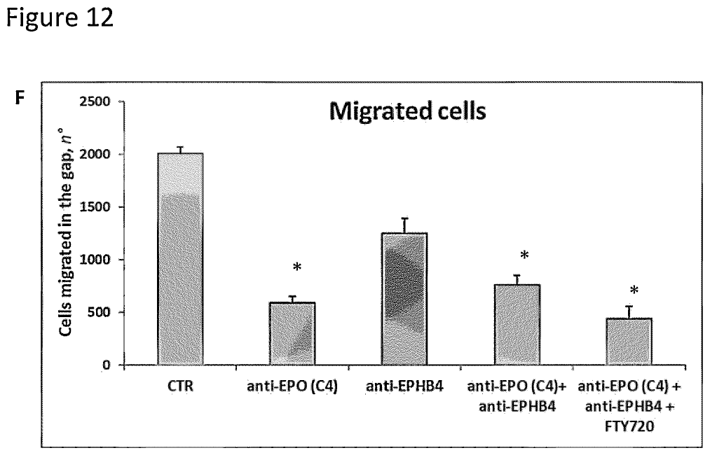

In addition, DLD1 cancer cells were treated with CTR (FIG. 12A), with anti-EPO (C4) alone (FIG. 12B), anti-EPHB4 (FIG. 12C), anti-EPO (C4)+anti-EPHB4 (FIG. 12D) or in combination with FTY720 (FIG. 12E). The treatment with anti-EPO (C4) with anti-EPHB4, showed a modest effect, 38% decrease. The co-administration of both mABs with FTY720 (FIG. 12E) increased the effects on migration inhibition up to 78%. (FIG. 12F). Data are expressed as the mean±standard deviation of at least 3 experiments in triplicate. *P<0.05; **P<0.01 versus CTR for all treatments tested.

### Example 7: Caspase Activity on Cancer Cell Line

Furthermore, cellular models were treated the above-mentioned pharmacological drugs and apoptotic effect was evaluated by caspase activity.

The analyses performed by Caspase Glo® 3/7 assay kit (Promega) revealed that anti-EPO mABs are able to induce caspase 3/7 activation, suggesting an increment of apoptosis of 200% for GSCs (FIG. 13A), 220% for GECs (FIG. 13B), 210% for GBM-CSCs (FIG. 13C), 205% for anaplastic astrocytoma cancer stem cells (FIG. 13D), 217% for DLD1 (FIG. 13E), 243% for PC-3 (FIG. 13F), 220% for LNCAP (FIG. 14A), 218% for MCF-7 (FIG. 14B), 180% for K562 (FIG. 14C), 234% for A2780 (FIG. 14D), 200% for A549 (FIG. 14E), and 200% also for SHSY-5Y (14F). Caspase activation was increased following combined treatments with anti-EPO (C4), FTY720, and/or TMZ by an average of 268% in all cancer models tested (FIGS. 13 and 14). Data are expressed as the mean±standard deviation of at least 3 experiments in triplicate. *P<0.05 versus CTR for all treatments tested.

Anti-EPO (C4) mAB, binding human EPO and/or negative functional modulators of the expression levels of EPO have proved to be effective molecules to be used in the treatment of cancer. This class of molecules proved to be effective both alone or in combination with functional modulators of sphingosine-1-phosphate (S1 P), FTY720, and or with functional modulators of EPO receptor, anti-EPHB4, through the inhibition of proliferation and angiogenesis both at cellular and functional level.

### Example 8: Analysis of the Effect of Treatment with Anti-EPO (C4) and Anti-EPHB4 on the Cell Viability of a Commercial Line of Microglia after Inflammatory Stimulus

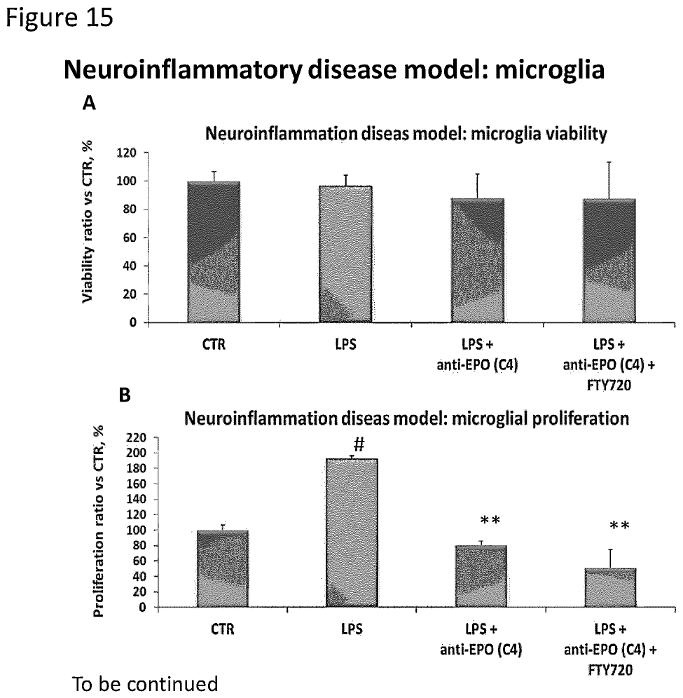

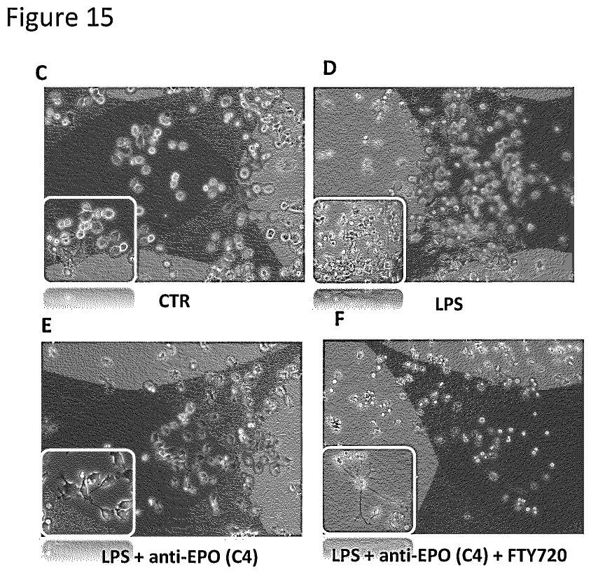

FIG. 15 shows cell viability of commercial line of microglia (line N9). N9 cells were cultured in Iscove's Modified Dulbecco's MEM, IMDM containing streptomycin/1× penicillin and 2 mM L-glutamine, supplemented with FBS (fetal bovine serum) at 5%. The cells were plated at a concentration of 1.5×10⁴ cells/cm₂ and kept in a thermostatic incubator at 37° C., with 5% CO₂, for 24 hours. The next day, the cells were exposed to different treatments for 48 hours. At the end of the treatments, cell viability was assessed by staining with trypan blue. The microglia was maintained in a basal culture medium and then activated with lipopolysaccharide (LPS), a molecule present on the membrane of the Gram-negative bacteria. Furthermore, to study the role of the product “anti-EPO (C4)” in the inhibition of neuroinflammation and then in the inhibition of the activation of microglia, the antibody according to the scheme below was administered, in association and not with LPS, to activate microglia.

The cells were exposed to the following treatments:
Control, CTR, (replacement of the culture medium at time 0 with fresh culture medium);
LPS, lipopolysaccharide, at a concentration of 3 μg/ml;
Anti-EPO (C4) (the culture medium is replaced at time 0 with fresh culture medium containing anti-EPO antibody);
LPS+anti-EPO (C4) (at time 0 the cells were plated in culture medium, activated with LPS at a concentration of 3 μg/ml, and treated with anti-EPO antibody (C4);

It is observed, surprisingly, that treatments with anti-EPO (C4) are not toxic for cells of quiescent microglia. The N9, in fact, after 48 hours of culture with different treatments retain a viability higher than 85% (FIG. 15A).

### Example 9: Analysis of the Effect of Treatment with Anti-EPO (C4) and Anti-EPHB4 on the Cell Proliferation of a Commercial Line of Microglia after Inflammatory Stimulus

N9 microglial cells were seeded in multiwell plates at a concentration of 1.5×10⁴ for 24 hours. The following day the cells were administered the following treatments for 24 hours:
LPS;
Anti-EPO (c4) antibody administered alone;
Anti-EPO (c4) antibody in combination with LPS
Anti-EPO (c4) antibody in combination with LPS and FTY720

N9 cells were detached with enzyme, and an aliquot of known volume was labeled with trypan blue and observed under the microscope for counting. Considering that the number of cells in the culture control, without any treatment, as being 100, the percentage of proliferation of N9 cultured in presence of LPS, anti-EPO (C4) antibody, combination of LPS and anti-EPO antibody was calculated (FIG. 15B). The data show (FIG. 15B) that the stimulus LPS markedly increases cell proliferation in response to inflammatory stimulus and, unlike treatment with anti-EPO (C4), is able to stop the proliferation of microglia following an inflammatory stimulus, maintaining the microglia in a state of quiescence, preventing the inflammatory cascade downstream such as release of inflammatory cytokines, nerve cell death and chronic inflammation. Surprisingly it was seen that even after microglial activation with LPS, the end result is an arrest of proliferation with values comparable to those of quiescent microglia (FIG. 15B). From a morphological analysis of N9 in basal condition (FIG. 15C), the stimulation with LPS (FIG. 15D), shows a change of the microglia from a branched morphology, typical of a quiescence state, to an amoeboid morphology, indicator of a phagocytic activity (FIG. 15D). Surprisingly, following treatment with anti-EPO (C4) cells recover or maintain a branched morphology (FIG. 15E), as well as after the combined treatment with anti-EPO (C4)+FTY720 (FIG. 15F). This result further emphasizes the effect that the anti-EPO (c4) antibody has against neuroinflammation, namely that of stopping the proliferation of the microglia not when it is in a state of resting, quiescence, but more when it is activated (FIG. 15). #P<0.05 versus CTR, **P<0.01 versus LPS treatment.

### Example 10: Analysis of the Effect of Treatment with Anti-EPO (C4) on Migration of a Commercial Line of Microglia after Inflammatory Stimulus

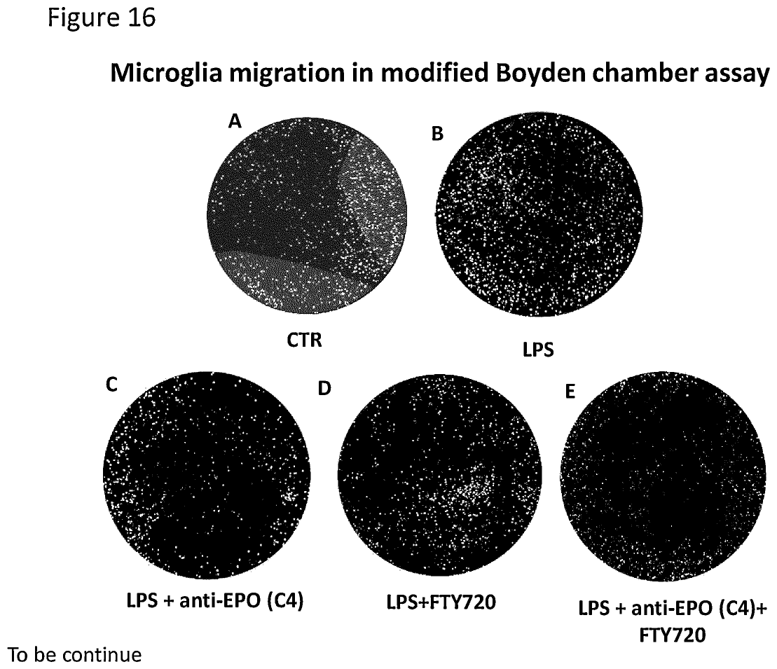

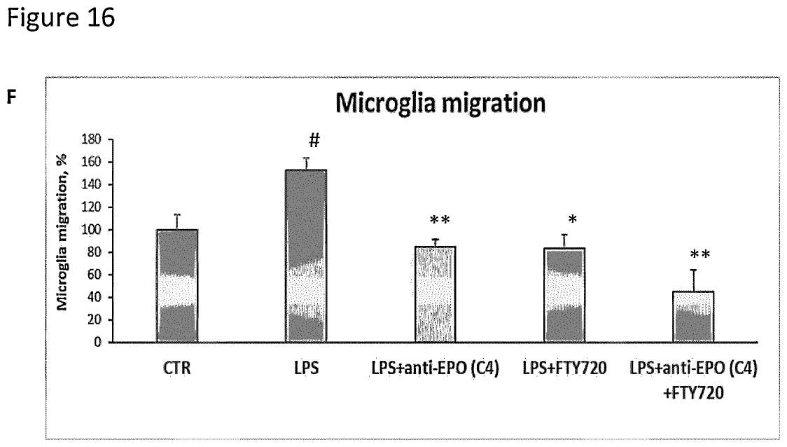

FIG. 16 shows the ability of treatment with anti-EPO and Fty720 to inhibit the migration of N9. The migration testing or “chemotaxis assay” was carried out to highlight the migratory capacity of N9, a phenomenon that is observed in response to an inflammatory stimulus. For this purpose, transwell multiwell plates (24-well) were used and equipped with inserts with a polycarbonate membrane. The holes in the membrane of a diameter of 8 μM are capable of retaining the cells and the culture medium, but allow the active transmigration of cells through the membrane to reach the lower well. The N9 treated were seeded in the top of the insert. In the lower compartments, BM (FIG. 16A) LPS at a concentration of 3 μg/mL in IMDM medium (FIG. 16B), and/or in combination with anti-EPO (C4) alone (FIG. 16C) or with FTY720 (FIGS. 16D and 16E) were added according to the scheme drawn. After 24 hours, the inserts were removed and the cells present in the lower compartment were stained with calcein AM. The visualization of the cells was made by fluorescence microscopy with a 4× objective and the images were analyzed with ImageJ software (FIG. 16). The migration testing shows that N9 cells are chemoattracted by the stimulus with LPS and that this effect is significantly reduced when in the medium of the lower compartment is added the anti-EPO (C4) antibody and, to an higher extent, when the cells are treated with anti-EPO (C4) and FTY720 (FIG. 16F). Therefore, it can be concluded that the anti-EPO (C4) treatment does not interfere with the quiescent microglia cells, but blocks their activation and migration, as a result of a potent inflammatory stimulus. Treatment with FTY720 has positive effects, and greater beneficial effects are observed with antibody anti-EPO (C4)+FTY720. #P<0.05 versus CTR, *P<0.05; **P<0.01 versus LPS treatment.

### Example 11: Analysis of the Effect of Treatment with Anti-EPO (C4) and Anti-EPHB4 on Viability and Proliferation of a Commercial Line of Microglia after Inflammatory Stimulus

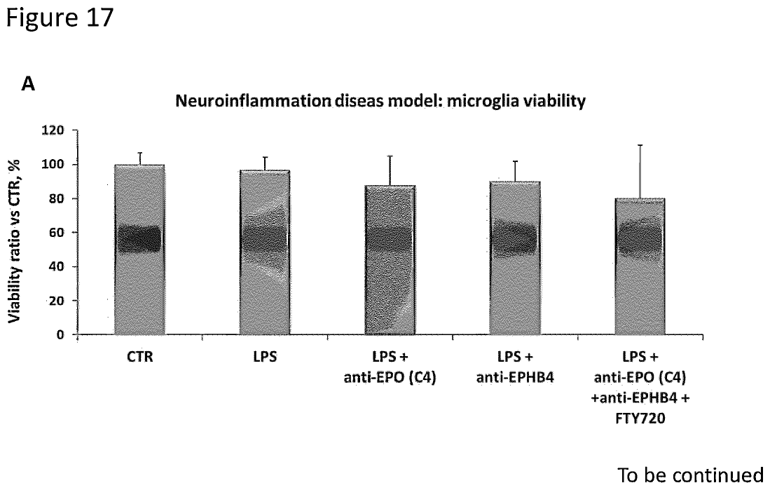

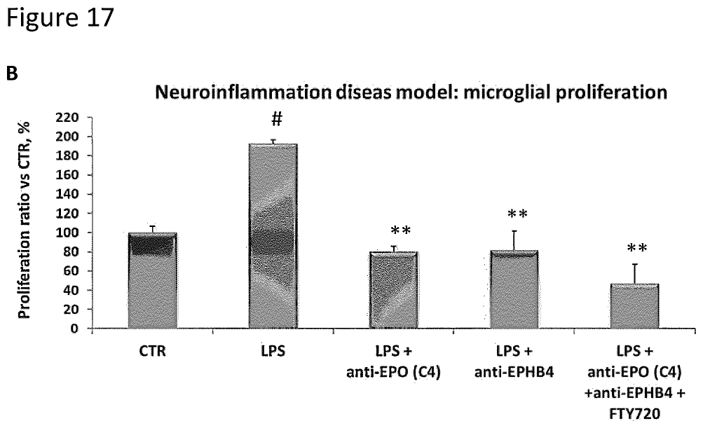

FIG. 17 shows cell viability (FIG. 17A) and proliferation (FIG. 17B) of commercial line of microglia (line N9). N9 cells were cultured in Iscove's Modified Dulbecco's MEM, IMDM containing streptomycin/1× penicillin and 2 mM L-glutamine, supplemented with FBS (fetal bovine serum) at 5%. The cells were plated at a concentration of 1.5×10\hat{ }4 cells/cm₂ and kept in a thermostatic incubator at 37° C., with 5% C02, for 24 hours, as above. The next day, the cells were exposed to different treatments for 48 hours. At the end of the treatments, cell viability and proliferation were assessed by staining with trypan blue and observed under the microscope for counting.

The microglia were maintained in a basal culture medium and then activated with lipopolysaccharide (LPS). The antibody according to the scheme below was administered, in association and not with LPS, to activate microglia.

The cells were exposed to the following treatments:
Control, CTR, (replacement of the culture medium at time 0 with fresh culture medium);
LPS, lipopolysaccharide, at a concentration of 3 μg/ml;
LPS+anti-EPO (C4) (at time 0 the cells were plated in culture medium, activated with LPS at a concentration of 3 μg/ml, and treated with anti-EPO antibody (C4);
LPS+anti-EPHB4 (at time 0 the cells were plated in culture medium, activated with LPS at a concentration of 3 μg/ml, and treated with anti-EPHB4);
LPS+anti-EPO (C4)+anti-EPHB4+FTY720

It is observed, surprisingly, that treatments with anti-EPO (C4) and anti-EPHB4 are not toxic for cells of quiescent microglia. The N9, in fact, after 48 hours of culture with different treatments retain a viability higher than 85% (FIG. 17A).

Similarly, to the treatment with anti-EPO (C4), a treatment condition of activated microglia with anti-EPHB4. In addition, the stimulus LPS markedly increases cell proliferation in response to inflammatory stimulus and, unlike treatments with anti-EPO (C4) and anti-EPHB4, are able to stop the proliferation of microglia following an inflammatory stimulus, also in combination with FTY720 (FIG. 17B). #P<0.05 versus CTR, **P<0.01 versus LPS treatment.

### Example 12: Analysis of the Treatments with Anti-EPO Individually and in Combination with FTY720 on Endothelial Cells Isolated from the Synovium of Hemophilic Patients

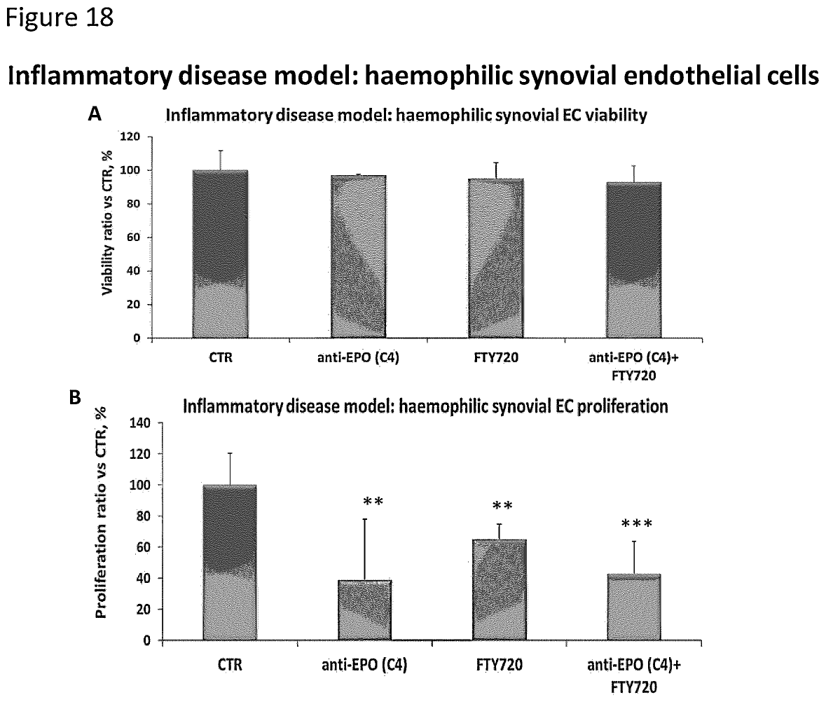

FIG. 18 shows the analysis of cell viability and proliferation of endothelial cells isolated from synovium of haemophilic patients (S-ECs) with moderate/severe cases of the disease. The endothelial cells were cultured in appropriate culture medium and subjected to the following treatments:
Control (CTR), replacement of the culture medium at time 0 with fresh culture medium.
administration of anti-EPO (C4), replacement of the culture medium at time 0 with fresh culture medium containing anti-EPO (C4) at a concentration of 6 μg/ml.
administration of FTY720, replacement of the culture medium at time 0 with fresh culture medium containing FTY720 (100 nM).
administration of anti-EPO (C4) in combination with FTY720, replacement of the culture medium at time 0 with fresh culture medium containing anti-EPO (C4) at a concentration of 3 μg/ml and FTY720 (1 μM).

It is observed, surprisingly, that treatments with anti-EPO (C4) and FTY720 are not toxic for endothelial cells isolated from the synovium of hemophilic patients. The S-ECs in fact, after 48 hours of culture with different treatments retain a viability higher than 88% (FIG. 18A). When endothelial cells are treated with the anti-EPO (C4) antibody, cell proliferation decreases significantly compared to placebo CTR, where the cells are maintained in their culture medium. In addition, the combined treatment with anti-EPO (C4) and FTY720 reduces further the survival of synovial endothelial cells of haemophilic patients (FIG. 18B). **P<0.01; ***P<0.001 versus LPS treatment.

Example 13: Analysis of the Treatments with Anti-EPO Individually and in Combination with FTY720 on the Functional Analysis of Cord Formation of Endothelial Cells Isolated from the Synovium of Haemophilic Patients

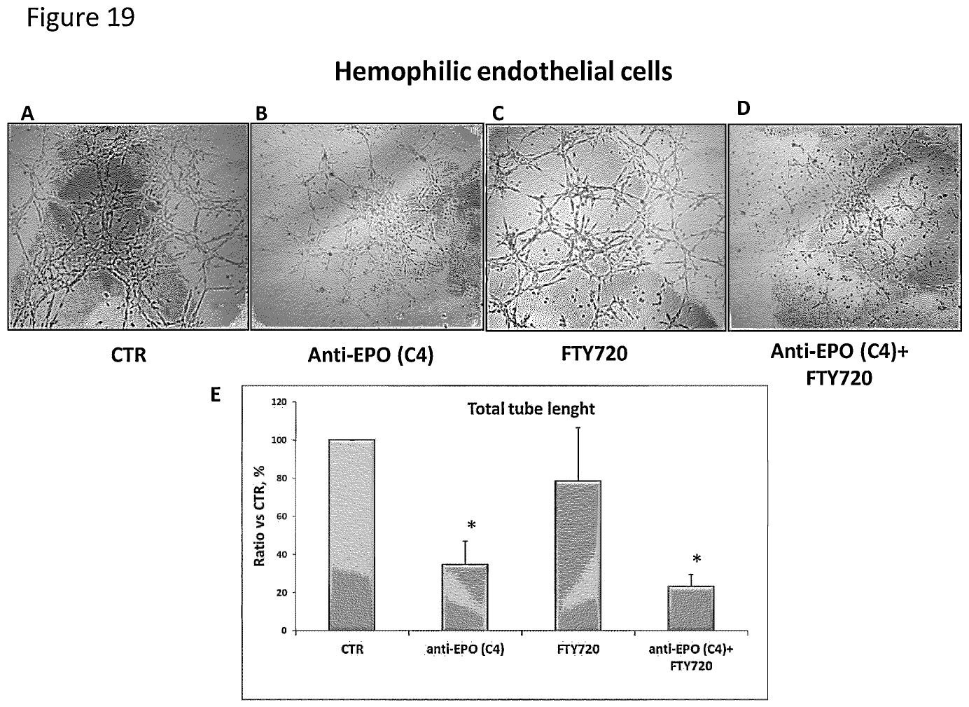

S-ECs (1×10⁴) were plated on 10 μL of Matrigel in BM, as a Control Condition (CTR, FIG. 19A) or in media containing the anti-EPO (C4) (FIG. 19B), FTY720 (FIG. 19C), and anti-EPO (C4)+FTY720 (FIG. 19D) treatments, as reported above. S-ECs were incubated at 37° C., 5% C02, 5% 02. After 48 h, tube-like structure formation was evaluated by phase-contrast microscopy. It was noted that the anti-EPO (C4) mAB was able to block the formation of tubular-like structures (FIG. 19B), while FTY720 (FIG. 19C) did not show such a significant effect. The total length of formed tubes in the assay, measured using the Angiogenesis Analyzer plugin in ImageJ, show a 65% decrease after administration of the anti-EPO (C4) treatment, which increase up to 76% when co-administered with FTY720 (FIG. 19E), indicating a more potent effect. Data are expressed as the mean±standard deviation of at least 3 experiments in triplicate. * P<0.05 versus CTR for treatments tested.

### Example 14: S1PR1 Expression on Endothelial Cells Isolated from the Synovium of Haemophilic Patients Rated with Anti-EPO (C4)

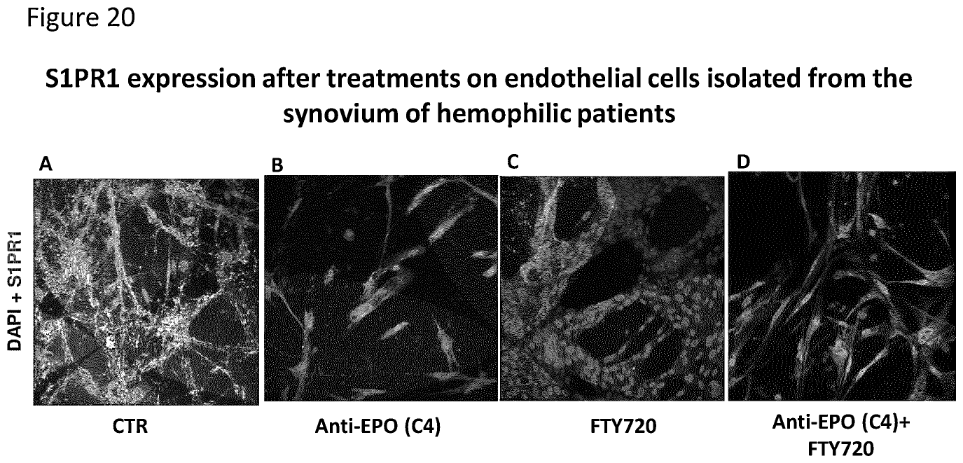

FIG. 20 shows S1 PR1 expression in S-ECs in CTR condition (FIG. 20A), after anti-EPO (C4) administration (FIG. 20B), or FTY720 alone (FIG. 20C) or in combination with anti-EPO (C4) (FIG. 20D). ECs (1×10⁴/well) were seeded into μ-Slide 8 Well, ibiTreat (Ibidi, Martinsried, Germany) collagen-coated. When cells reached the desired confluence, were fixed in paraformaldehyde 4% for 20 min at RT, washed twice with D-PBS and incubated with 0.1 M glycine to quench auto-fluorescence. Then, the coverslip was incubated with PBS+0.25% Triton ×100 to permeabilize cell membranes and then blocked in PBS+5% BSA for 30 min at RT. Incubation with anti-S1 PR1 primary antibody diluted in blocking buffer was performed overnight at 4° C. The following day, primary antibody was removed and fluorescent secondary antibody labelling was then added for 45 min at RT, protected from light, washed and finally, the coverlips were mounted with ProLong Gold Antifade Mountant (ThermoFisher). Immunolabeling was acquired using an inverted DM14 microscope equipped with DFC350×CCDcamera and LAS-X software (all from Leica Microsystems, Wetzlar, Germany). Results show that the treatment with anti-EPO (C4) surprisingly decreased expression levels of S1 PR1 within the endothelial cells of the pathological synovium (FIG. 20). S1 PR1 levels are increased in pro-tumoral and pro-angiogenic conditions, when intracellular and extracellular levels of S1 P are presented. It is possible therefore to hypothesize the use of the anti-EPO (C4) antibody, such as in direct intra-articular treatment in the form of gel or suspension, in association or not with “coagulation factors and their derivatives” and also comprising FTY720 and negative modulators of the sphingosine-1-phosphate pathway.

### Example 15: Analysis of the Treatments with Anti-EPO Individually and in Combination with Anti-EPHB4 on Endothelial Cells Isolated from the Synovium of Haemophilic Patients

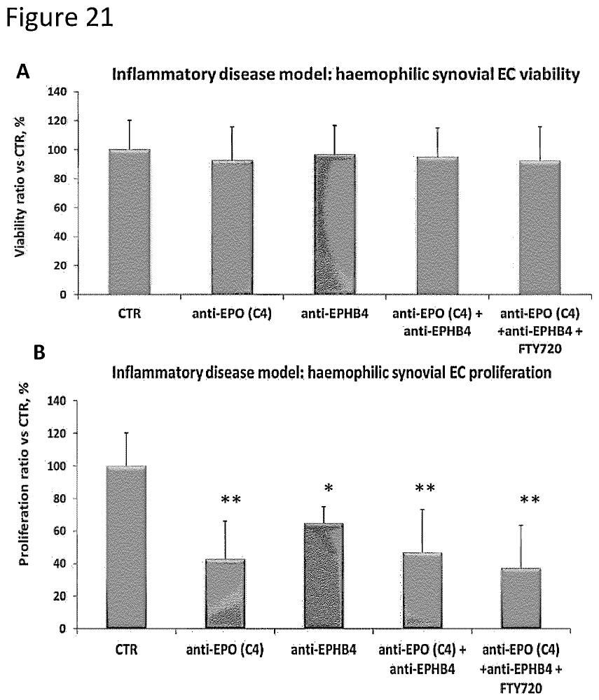

FIG. 21 shows the analysis of cell viability and proliferation of endothelial cells isolated from synovium of haemophilic patients (S-ECs) with moderate/severe cases of the disease. The endothelial cells were cultured in appropriate culture medium and subjected to the following treatments:
Control (CTR), replacement of the culture medium at time 0 with fresh culture medium.
anti-EPO (C4), replacement of the culture medium at time 0 with fresh culture medium containing anti-EPO (C4) at a concentration of 6 μg/ml.
anti-EPHB4, replacement of the culture medium at time 0 with fresh culture medium containing anti-EPHB4
anti-EPO (C4) in combination with anti-EPHB4, replacement of the culture medium at time 0 with fresh culture medium containing anti-EPO (C4) at a concentration of 3 μg/ml and anti-EPHB4
anti-EPO (C4) in combination with anti-EPHB4 and FTY720, replacement of the culture medium at time 0 with fresh culture medium containing anti-EPO (C4) at a concentration of 3 μg/ml and anti-EPHB4, and FTY720 at a concentration of 100 nM.

It is observed, surprisingly, that treatments with anti-EPO (C4) and anti-EPHB4 alone or in combination with FTY720 are not toxic for endothelial cells isolated from the synovium of hemophilic patients (FIG. 21A). The S-ECs in fact, after 48 hours of culture with different treatments retain a viability higher than 90% (FIG. 21A). When endothelial cells are treated with the anti-EPO (C4) antibody, cell proliferation decreases significantly compared to placebo CTR, where the cells are maintained in their culture medium. In addition, the combined treatment with anti-EPO (C4), anti-EPHB4 and FTY720 reduces further the survival of synovial endothelial cells of haemophilic patients (FIG. 21B). * P<0.05; **P<0.01 versus CTR for treatments tested.

### Example 16: Gene Expression of HOC84 Stem Cells

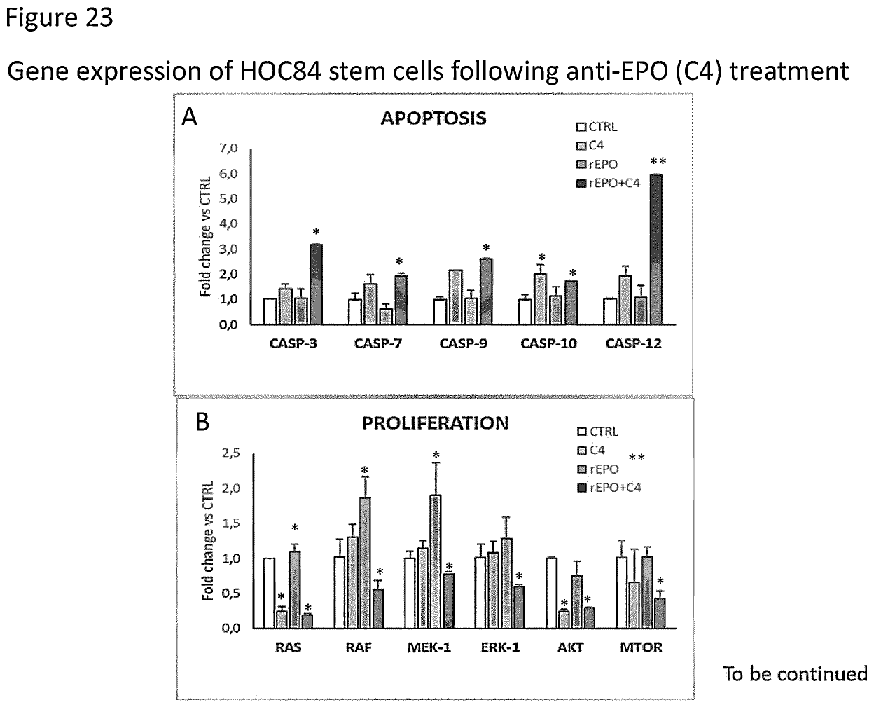

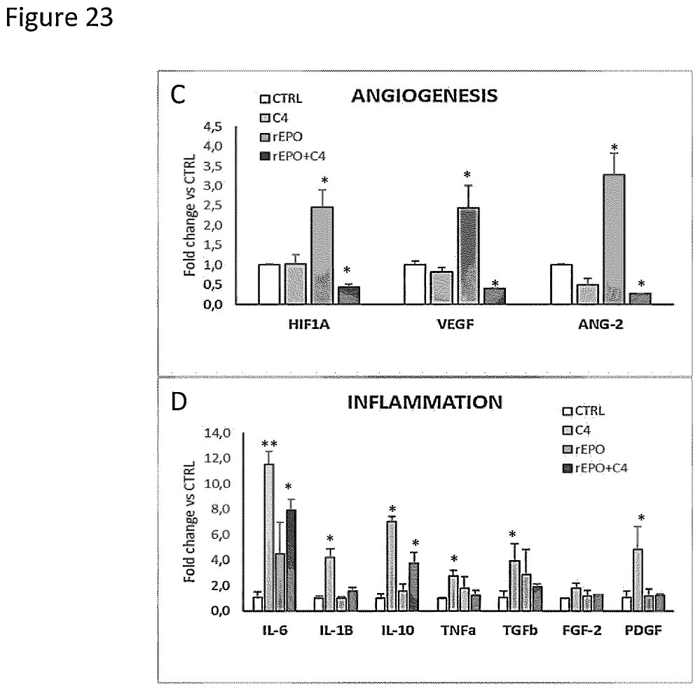

The analysis was performed by the assessment of gene expression by RealTime PCR on genes related to apoptosis (FIG. 23A), proliferation (FIG. 23B), angiogenesis (FIG. 23C), and inflammation (FIG. 23D). HOC84 stem cells (2×10⁵) were seeded into 25 cm2 collagen-coated culture flasks. When 90% confluence was reached, cell cultures were added with anti-EPO(C4) and/or recombinant human EPO (rEPO), for 72 h. At the end, total RNA was extracted following TRI-Reagent protocol and quantified with NanoDrop 1000 Spectro-photometer (Thermo Fisher Scientific). Reverse transcriptase reaction was executed using TranScriba Kit (A&A Biotechnology), loading 1 μg of RNA (A260/A280>1.8), according to manufacturer's instructions. qRT-PCR was performed using StepOnePlus™ (Thermo Fisher Scientific), 1 μg of cDNA, forward and reverse primers (250 nM each) Titan HotTaq EvaGreen® qPCR Mix (Bioatlas). Data were normalized to 18S expression, used as endogenous control. Relative gene expression was determined using the 2−ΔΔCt method (FIG. 23). Results demonstrated that the treatment with anti-EPO (C4) in co-administration with rEPO showed the most potent effect in inducing apoptosis (FIG. 23A). Genes related to proliferation were significant more expressed following rEPO treatment, and their expression was inhibited following administration of anti-EPO (C4) (FIG. 23B), as well as for genes related to angiogenesis (FIG. 23C). Interestingly, genes related to inflammation were higher expressed after anti-EPO (C4) treatment (FIG. 23D). All in all, gene expression on HOC84 following the above-mentioned treatments indicated that anti-EPO (C4) significantly inhibits proliferation and angiogenesis, and increases apotosis and inflammation, counteracting the effect of rEPO (FIG. 23).

### Example 17: Anti-EPO (C4) Treatment Sensitizes Chemo-Resistant Cancer Stem Cells to Anti-Tumor Treatment

To confirm the effect of anti-EPO (C4) on chemo-resistant cells, glioma cell line (GSC and U87) and ovarian cancer cells HOC84 were treated with anti-EPO (C4) alone or in combination with chemotherapeutics (TMZ or CPT). In detail, GSCs and U87 parental cell line, which are sensitive to TMZ, were first maintained in low doses of TMZ (25 μM) and then successively exposed for two months to incremental doses of 25 μM of TMZ each time (up to 500 μM). After the killing of a majority of the cells, the surviving cells were maintained until a normal rate of growth were obtained. HOC84, which are naturally resistant to carboplatin (CPT), were cultured for 96 h in basal condition supplemented with 40 μg/mL of CPT. Cell viability was assessed by 3-(4,5-Dimethylthiazol-2-yl)-2,5-Diphenyl-tetrazoliumBromide (MTT) assay, as a function of redox potential (FIG. 24). Cells (5×103/well) were seeded and cultured in 96-well plate for 24 h in Basal Medium (BM). Then, culture media were replaced with fresh media containing the specific treatments, or in BM as a control condition (CTR).

Test was performed in triplicate after 96 h of treatment, by replacing culture media with 100 μL of fresh media added with 10 μL of MTT 5 mg/ml in D-PBS. After 4 h of incubation, media were removed and cells were lysed with 100 μL of 2-propanol/formic acid (95:5, by vol) for 10 min. Then, absorbance was read at 570 nm in microplate reader. GSCs-R cells showed a resistance response following TMZ treatment (100 μM, which represents the clinically relevant concentration of the drug) and a significant decrease in percent survival from approximately 93% to 40% whereas in U373 cells the decrease was from 100% to 30% with anti-EPO (C4) and an increasing mortality following anti-EPO (C4) in combination with TMZ up to 19% of viability (FIG. 24A, **p<0.01). U87-R cells showed a decrease of viability from 100% to 28% in response to treatment with anti-EPO (C4), with an increase in mortality up to 18% following anti-EPO (C4) in combination with TMZ (FIG. 24B, **p<0.01). Interestingly, anti-EPO (C4) increase mortality in HOC84 stem cells both in single administration (66%) or in combination with cisplatin (CPT) up to 70% (FIG. 24C, **p<0.01). Surprisingly it was seen that the anti-EPO antibody can be used in the treatment of patients which are resistant to chemotherapeutic standard treatments with at least one antitumor agent or wherein the treatment with an antitumor agent should be avoided.

### Example 18: Mapping of Anti-EPO (C4) with Human EPO

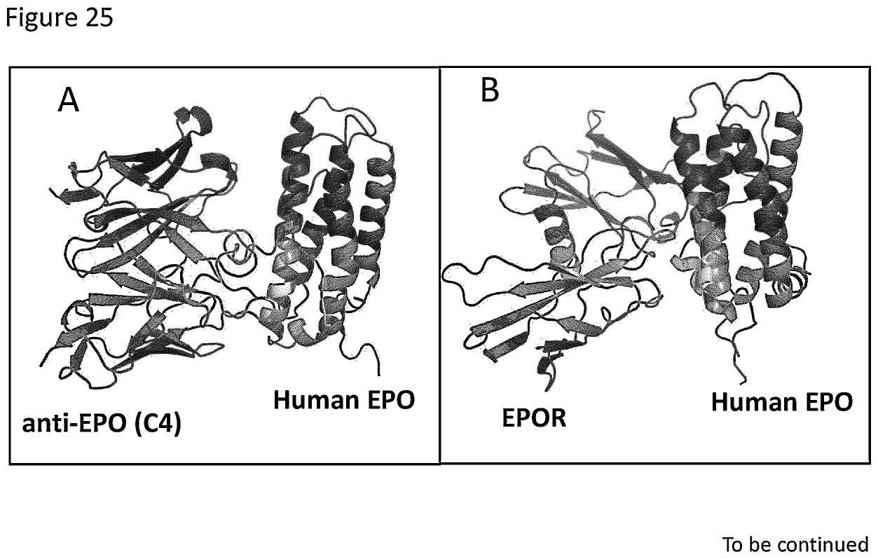

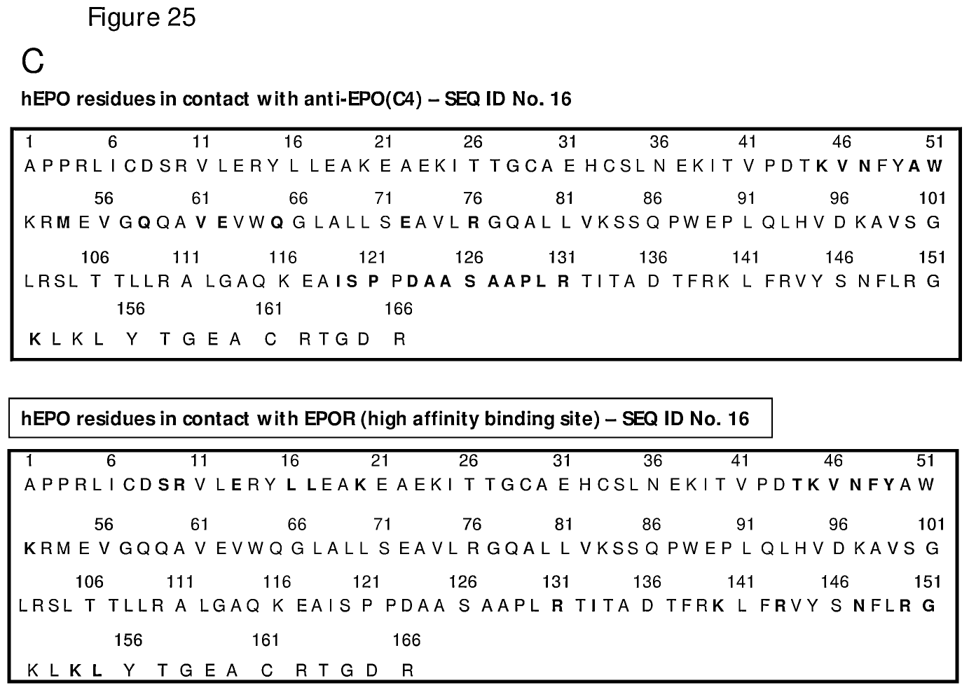

In order to investigate hEPO amino acid residues that participate in the antigen-antibody recognition, we used the experimentally resolved structure of hEPO (pdb: 1 buy) and the structure of anti-EPO-C4 predicted by IgFold, following the methodology described by Ruffolo et al., (Ruffolo, J. A., Chu, L.-S., Mahajan, S. P. & Gray, J. J. Fast, accurate antibody structure prediction from deep learning on massive set of natural antibodies. 2022.04.20.488972 (2022) doi:10.1101/2022.04.20.488972). After modeling, the ClusPro2.0 server (htts://cuspro.bu.edu/) was used to predict the interactions between modeled anti-EPO (C4) and hEPO. The antibody mode was selected with the non-CDR regions masked automatically. ClusPro selected the 1000 best scoring solutions, clustered them according to Root Mean Square Deviation (RMSD) considerations, and the lowest ClusPro score, representing the greatest probability of antigen-antibody interaction, was selected. The most probable binding complex based on docking is shown in FIG. 25A, where the residues at the interface (distance lower the 4.6 Angstroem) are colored in red. The anti-EPO (C4) binds in similar region like EPOR (FIG. 25B, pdb:1eer). Parts of the residues in contact with anti-EPO (C4) antibody overlap with residues of the high affinity binding site of the hEPO/EPOR complex (FIG. 25C), which suggests a neutralizing activity of anti-EPO (C4).

### Example 19: Pharmacokinetic Analysis of Anti-EPO (C4) by Surface Plasmonic

Resonance Technology

SPR is a technology widely used to study in real time the interaction between two unlabeled molecules, one (the “ligand”) immobilized on a sensor chip, and the other (the “analyte”) flowing through a microfluidic system over the chip surface. Binding is measured in real time as a change in the refractive index on the surface. The most common application of SPR is to determine the association/dissociation binding constants for biomolecular interactions, but its versatility allows many other uses, including label-free immunoassays and concentration determination of biologics.

The study was carried out using an up-to-date SPR apparatus, the ProteOn XPR36 Protein Interaction Array system (Bio-Rad Laboratories). This system is mainly characterized by the presence of six flow channels which can uniformly immobilize up to six ligands on parallel strips of the same sensor surface.

The flow channels can be rotated 90° so that up to six analytes, or six concentrations of the same analyte, or six plasma samples can be flowed in parallel, creating a 36-spot interaction array.

The ligands hEPO and BSA (Sigma-Aldrich, Italy, used as reference) were immobilized using amine-coupling chemistry on parallel channels of a CMD700L (Xantec GmbH) sensor chip. Briefly, surface was activated with sulfo NHS/EDC according to manufacturer's recommendation; hEPO and BSA were diluted at a concentration of 400 and 30 μg/mL in acetate buffer, pH 4.0 and 4.5, respectively. These solutions were flowed for 5 min at a rate of 30 μL/min over the activated chip surface. The remaining activated groups were blocked with ethanolamine, pH 8.0.

The analyte solutions (e.g. control plasma containing spiked anti-EPO (C4) antibody, or plasma/tissue samples from treated mice) were injected so that they flowed simultaneously on both immobilized hEPO and the reference surface. Dissociation was measured in the following 10 minutes. All of these assays were carried out at 25° C. The sensorgrams (time course of the SPR signal in RU) were normalized to a baseline value of 0. The signals observed in the surfaces immobilizing hEPO were corrected by subtracting the nonspecific response observed in the reference surfaces.

The following buffer was used to reduce the non-specific binding (NSB): 75 mM Tris buffer containing 450 mM NaCl, 1 mM EDTA and 0.005% Tween 20 (TBST pH 7.0). In this manner, we could use plasma samples diluted as low as 10-fold. We used 50 mM HCl to regenerate the chip surface.

We found that EPO could be well immobilized on SPR sensor chip and that the subsequent flow of plasma spiked with different concentrations of anti-EPO (C4) antibody results in concentration-dependent SPR binding signals at the end of the dissociation phase. This method can thus allow to determine the plasma and tissue concentrations of antibody, on the basis of an appropriate calibration curve (FIG. 26A). The Lower Limit of Detection calculated was ˜0.5 μg/mL (˜3.3 nM), which corresponding to a LLD of 5 μg/mL in undiluted plasma.

Tumor tissues were analyzed after their homogenization in TBST (1 g/2 mL) with an Precellys ysing Kit tubes, ultracentrifugation for 1 h at 110000 g and 1:2 dilution of the supernatant into optimized TBST buffer.

The pharmacokinetic (PK) profile of anti-EPO (C4) was assessed after single i.v. injection in mice models for glioblastoma. In particular, the antibody concentrations were measured at different time points after treatment (from 6 hours to 1 week) in:

1) plasma, to determine the main PK parameters (mainly half-time of elimination and volume of distribution); 2) tumor, to assess the antibody presence in the target tissue; 3) in kidney and liver to evaluate the elimination route.

When tumor masses reached about 500 mg, animals were treated i.v. with 10 mg/kg of anti-EPO (C4) antibody. Blood samples were collected from the retroorbital plexus under isoflurane anesthesia at 4, 24, 48, 72, 120, and 168 h from treatment (FIG. 26B). Mice were sacrificed by cervical dislocation. Tumors were collected. To obtain plasma, blood samples were centrifuged at 4000 rpm for 10 min at 4° C. All samples were immediately frozen in dry ice and then stored at −20° C. until analysis. Four mice were used at each time point.

The pharmacokinetic profile in plasma was studied, and the results showed that the anti-EPO (C4) was stable in circulation with a half-time of elimination of about 4.4 days after single administration (FIG. 26B). In addition, SPR analysis was performed for anti-EPO (C4) to evaluate the binding kinetics parameters. The sensorgrams obtained on the anti-EPO (C4) immobilized with amine coupling were analyzed and the fitting results indicated the following parameters of anti-EPO (C4) for human EPO (h-EPO) ka=3.15E+0.3(1/Ms), kd=1.57E-04(1/s), KD=4.97E-08(M). We than performed a comparative analysis to evaluate the affinity of anti-EPO (C4) for human or murine EPO. Notably, the representative sensorgrams (FIG. 26C) clearly revealed differences in their affinity. Indeed, h-Epo has an affinity 20 times higher than m-Epo (KD=1000 nM vs 50 nM; m-EPO: ka=6.63E+0.2(1/Ms), kd=6.69E-04(1/s), KD=1.01 E-06(M) (FIG. 26C).

Interestingly, the analysis of the tumor tissues revealed that anti-EPO (C4) levels were measurable, with a bell-shaped PK profile, similar to that of antibodies already used in the clinic (e.g. trastuzumab) (FIG. 26D). Evaluating binding kinetic parameters, we noted that the constant of dissociation KD, was in line with recent publications reporting that monoclonal antibodies with KD values in the order of nM have a greater efficiency of penetration into tumor tissue and can distribute more efficiently within tumor tissues. Indeed, as reported above, when we evaluated anti-EPO distribution, we measured higher levels of antibody in the tumoral tissues respect to kidney and liver (FIG. 26D), indicating a more specific uptake of anti-EPO (C4) in tumor tissues.

### Example 20: Analysis of the Hematological, Liver and Renal Toxicity In Vivo, Following Anti-EPO (C4) Administration

Blood samples (100 μL) were collected into K3EDTA coated tubes, which were placed on a rotary mixer for at least 30 min, then analyzed for hematological analysis.

Blood count was measured following 18 days after treatment starting, revealing no significant variations in all hematological parameters analyzed after anti-EPO (C4) administration. FIGS. 27 and 28 refer to blood count of mice treated intravenously (IV) for 18 days (T18) with anti-EPO at 10 mg/Kg. (A) RBC: red blood cells; (B) HGB: hemoglobin; (C) HCT: hematocrit; (D) MCV: mean corpuscular volume; (E) MCH: mean corpuscular hemoglobin; (F) MCHC: mean corpuscular hemoglobin concentration; (G) RDW: red cell distribution width; (H) RET: reticulocyte Count; (I) PLT: platelets. Data are means±SD (FIG. 27) and A) WBC: white blood cells; (B) NEUT: neutrophils; (C) LYMP: lymphocytes; (D) MONO: monocytes; (E) EOS: eosinophils; (F) BASO: basophils. Data are means±SD (FIG. 28).

Serum for Biochemistry were collected at least 400 μL of blood and putted it in an eppendorf tube with or without (plasma/serum) anticoagulant. The samples were left at environment temperature for at least 30′ minutes, then centrifuged at 500 g for 10 min, the supernatants were collected, being careful not to take the precipitate and analyzed for Biochemical tests. Biochemistry analysis was measured at two time points, 11 and 18 days after treatment starting, revealing no significant variations after anti-EPO administration at different doses and at different timepoint. (A) Urea; (B) Creatinine; (C) Albumine, (D) AST; aspartate aminotransferase; (E) ALT: alanine aminotransferase. (FIG. 29)

### Example 21: Gene Expression of Cell Derived Xenograft GSCs after In Vivo Treatment with Anti-EPO (C4)

The analysis was performed by the assessment of gene expression profile on human tumor GSC-derived xenograft. Gene expression analysis were conducted by Real-Time PCR, run in triplicate, using 18S as endogenous control. Samples have been normalized to untreated CTRL. The analysis was conducted on genes related to FIG. 30A apoptosis, FIG. 30B inflammation, FIG. 30C proliferation.

Tissues were processed and total RNA was extracted following TRI-Reagent protocol and quantified with NanoDrop 1000 Spectro-photometer (Thermo Fisher Scientific). Reverse transcriptase reaction was executed using TranScriba Kit (A&A Biotechnology), loading 1 μg of RNA (A260/A280>1.8), according to manufacturer's instructions. qRT-PCR was performed using StepOnePlus™ (Thermo Fisher Scientific), 1 μg of cDNA, forward and reverse human primers (250 nM each) Titan HotTaq EvaGreen® qPCR Mix (Bioatlas). Data were normalized to 18S expression, used as endogenous control. Relative gene expression was determined using the 2-ΔΔCt method (FIG. 30).

### Example 22: Diagnostic Panel Performed by Genetic and Protein Expression Analysis

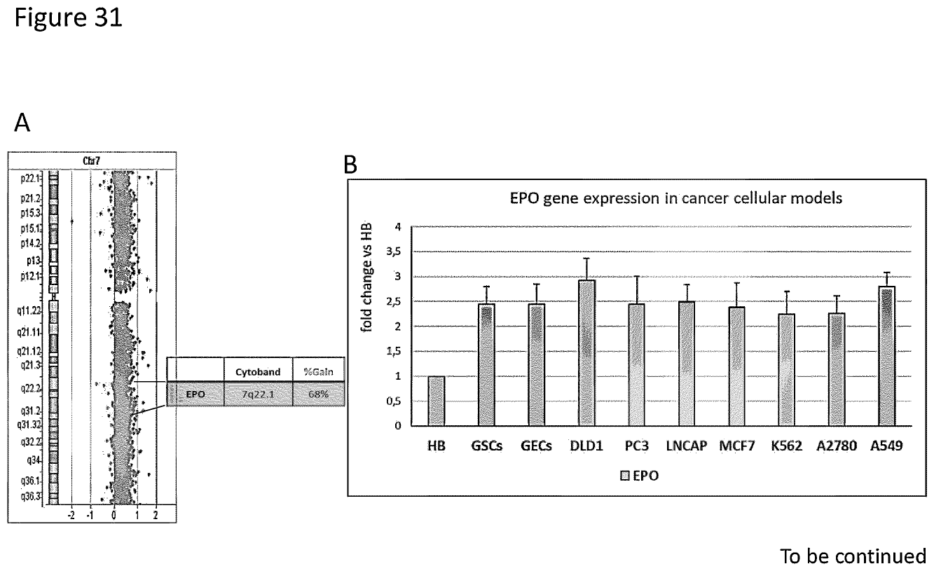

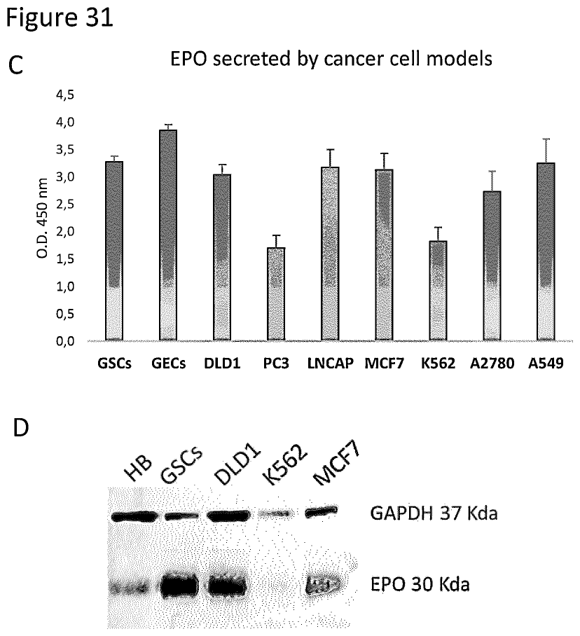

In order to provide a method of detecting the presence of human erythropoietin in a biological samples, we created a diagnostic panel based on genetic and molecular analysis, in which anti-EPO (C4) is the antibody used to perform the analysis. In detail the diagnostic panel foresees a multi-level analysis, from genetics, gene expression to protein expression analysis. The results of the analysis of chromosomal alterations such as Copy Number Variation (CNV) showed that in the tumor tissues of brain neoplasia there is a chromosomal imbalance in favor of the genes of the EPO signaling pathway. In particular, in tumor biopsies from patients affected by GBM, the EPO, gene (FIG. 31A) was amplified in 68% of the tumor biopsies. Genomic DNA was extracted from tissue samples using the QIAamp Fast DNA Tissue Kit according to the indicated protocol (Qiagen). The DNA was quantified using a NanoDrop ND-1000 spectrophotometer (ThermoFisher Scientific) and the integrity was assessed by microcapillary electrophoresis on 2100 Bioanalyzer 2100 (Agilent Technologies). High resolution CNV analysis was performed using an 8×60K array platform (Agilent Technologies). Interpretation of results was done using Feature Extraction software and Agilent CytoGenomics v. 4.0.3.12 and Genomic Workbench v. 7.0.4.0 (Agilent Technologies). The raw data were analyzed using the Cytogenomics software with the ADM-2 algorithm (the positions of the breakpoints were reported according to the reference genome GRch37/hg19). A minimum of three consecutive probes/regions was considered as a filter. Gene expression analysis revealed that EPO is overexpressed in all cancer cell models analyzed, such as glioblastomas, colon cancer, prostate cancer, myeloid leukemia, breast cancer, ovarian cancer, and lung cancer (FIG. 31B). anti-EPO (C4) was further used to set-up an ELISA test (FIG. 31C).

Briefly, 96-well microtiter plate were washed with buffer, than wells were pre-coated with capture anti-EPO antibody, then 100 μL of each standard and sample were added into appropriate wells overnight at 4° C. with gentle shaking. The following day solutions were discarded, wells were washed and coated with Biotinylated anti-EPO antibody. The wells were sealed with adhesive cover, incubate at room temperature for 1 hour on shaker, washed with wash buffer. Then, streptavidin-HRP Conjugate was added to each well and incubated at room temperature for 15 minutes on shaker. Plates were washed with wash buffer and then TMB Substrate Solution was added. After 15 minutes of incubation at room temperature, stop solution (0.5 N sulphuric acid) was added to each well and data were acquired by measuring light absorption at 450 nm. Results, obtained by plotting the measured values in a 5-parametric curve, were reported in FIG. 31C. Lastly, anti-EPO antibody was used for Western Blot analysis in order to evaluate protein expression. In detail, CSCs from Glioblastoma, colon cancer (DLD1), myelogenous leukemia (K562), and breast cancer (MCF7) (2×10⁵) were seeded into a 25 cm² culture flasks and cultured until they reached the appropriate confluence (about 80%-90%). Then, cells were lysed with M-PER Protein Extraction Reagent (Thermo Fisher Scientific) in presence of Halt Protease Inhibitor Cocktail (Thermo Fisher Scientific). Proteins were quantified by the Pierce Detergent Compatible Bradford Assay Kit (Thermo Fisher Scientific). Protein lysates (30 mg) were separated in Bolt 10% Bis-Tris Plus Gels (Thermo Fisher Scientific) in Mini Gel Tank (Thermo Fisher Scientific) and transferred onto nitrocellulose iBlot 2 Transfer Stacks using iBlot 2 Dry Blotting System (Thermo Fisher Scientific). After transfer, the membrane was blocked in Tris-buffered saline/Tween 20 b 5% milk solution and incubated separately with anti-GAPDH (SantaCruz Biotechnology), anti-EPO (C4) overnight at 4° C. After incubation with HRP-labeled secondary antibody (Invitrogen, Carlsbad, California, USA), protein bands were scanned with SuperSignal West Pico PLUS Chemiluminescent Substrate (Thermo Fisher Scientific) and detected by ChemiDoc XRSO (Bio-Rad, Hercules, California, USA). Densitometric analysis were performed using ImageJ. (FIG. 31D).

From the above description and the above-noted examples, the advantage attained by the anti-EPO antibody described and obtained according to the present invention are apparent.

1. An isolated anti-Erythropoietin (EPO) antibody, wherein said antibody comprises:
a. a variable domain of a light chain (VL) having the amino acid sequence of SEQ ID NO:6; and
b. a variable domain of a heavy chain (VH) having the amino acid sequence of SEQ ID NO:14.

2. The isolated anti-EPO antibody of claim 1, wherein said antibody comprises 6 CDR regions, said CDR regions being:
a. a VL-CDR1 having the amino acid sequence of SEQ ID NO:4;
b. a VL-CDR2 having the amino acid sequence of GAS (Gly-Ala-Ser);
c. a VL-CDR3 having the amino acid sequence of SEQ ID NO:5;
d. a VH-CDR1 having the amino acid sequence of SEQ ID NO:11;
e. a VH-CDR2 having the amino acid sequence of SEQ ID NO:12; and
f. a VH-CDR3 having the amino acid sequence of SEQ ID NO:13.

3. The isolated anti-EPO antibody of any one of claims 1 or 2, wherein the antibody is a monoclonal antibody, a chimeric antibody and/or is humanized or human.

4. The antibody of any one of claims 1-3, wherein the antibody further comprises a framework sequence and at least a portion of the framework sequence is a human consensus framework sequence.

5. The antibody of any one of claims 1-4, which is a full-length monoclonal antibody.

6. The antibody of any one of claims 1-4, wherein the antibody is bispecific.

7. A polynucleotide encoding the antibody of any one of claims 1-6.

8. A vector comprising the polynucleotide of claim 7, wherein the vector is optionally an expression vector.

9. A host cell comprising the vector of claim 8.

10. The host cell of claim 9, wherein the host cell is prokaryotic, eukaryotic, or mammalian.

11. An immunoconjugate comprising the antibody of any one of claims 1-6 conjugated to an agent, wherein said agent is chosen from the group consisting of a drug or cytotoxic agent. or co-administered in combination with a negative functional modulator of S1 P signaling, and/or anti-EPO receptors selected from the group comprising EPOR, EPHB4, CSFR2B, tissue protection factor (TPR, EPOR/CD131 heterodimer), and/or EPO mimetics.

12. A method for producing the anti-EPO antibody of any one of claims 1-6 or the immunoconjugate of claim 11, said method comprising (a) expressing the vector of claim 8 in a suitable host cell, and (b) recovering the antibody or immunoconjugate.

13. The method of claim 12, wherein the host cell is prokaryotic or eukaryotic.

14. A pharmaceutical composition comprising (i) the anti-EPO antibody of any one of claims 1-6 or (ii) the polynucleotide of claim 7, wherein the composition optionally further comprises a carrier.

15. The composition according to claim 14, for oral, or parenteral, topical, rectal, intravenous, subcutaneous, intramuscular, intranasal, intravaginal, intravitreal, through the oral mucosa, the lung mucosa, or for transocular administration.

16. The composition according to claim 14, wherein said pharmaceutical composition is administered incorporated into liposomes, microvescicles, bound to molecular carriers or combined with molecules selected from the group consisting of molecules that allow the temporary opening of the blood-brain barrier, anti-inflammatory molecules, monoclonal antibodies and drugs with immunosuppressive activity.

17. The antibody of any one of claims 1-6, the composition of claim 14 or the immunoconjugate of claim 11 for use as a medicament.

18. The antibody, composition, or immunoconjugate for use according to claim 17, wherein said antibody, composition, or immunoconjugate is for use in the treatment of a tumor, cancer, or cell proliferative disorder, and/or for inhibiting angiogenesis or vascular permeability, of autoimmune and non-autoimmune based chronic inflammatory diseases, in the treatment of patients undergoing organ or tissue transplant, in the treatment of haemophilic arthropathy, neurodegenerative diseases and neurological diseases in which neuro inflammation plays a role in pathogenesis, such as multiple sclerosis, Parkinson's disease, Alzheimer's disease, frontotemporal dementia, dementia with Lewy bodies, autoimmune disease with neurologic involvement, Amyotrophic Lateral Sclerosis, and Neuromuscular Diseases, ophthalmic pathologies such as neovascular age related (NVAMD), macular degeneration, retinal vein occlusion (RVO), metabolic syndromes, diabetes, and neuropathic pain disorders

19. The antibody, composition, or immunoconjugate for use according to claim 18, wherein said tumor, cancer, or cell proliferative disorder is chosen from the group consisting of cerebral astrocytoma, cerebellar astrocytoma, astrocytoma of the pineal gland, oligodendroglioma, pituitary adenoma, craniopharyngioma, sarcoma, glioblastoma grade II fibrillary astrocytoma, protoplasmic, grade III gemistocytic, anaplastic astrocytoma, including gliomatosis cerebri, pituitary adenoma, ependymoma, medulloblastoma, neural ectoderm tumor, neuroblastoma, hypothalamic glioma, breast cancer, lung cancer, colon cancer, cervical cancer, endometrial cancer, uterine cancer, ovarian cancer, esophageal cancer, basal cell carcinoma, cholangiocarcinoma, cancer of the spleen, osteosarcoma, intraocular melanoma, retinoblastoma, stomach cancer, heart cancer, liver cancer, hypopharyngeal cancer, laryngeal cancer, cancer of the oral cavity, nasal and paranasal cancer, cancer of the salivary glands, nasopharyngeal cancer, throat cancer, thyroid cancer, pancreatic cancer, kidney cancer, prostate cancer, bladder cancer, rectal cancer, testicular cancer, renal cell cancer melanoma, sarcoma, mesothelioma, pheochromocytoma, and hematological cancers.

20. The antibody, composition, or immunoconjugate for use according to any one of claims 17-19, wherein said tumor, cancer, or cell proliferative disorder is chosen said cancer is selected from the group consisting of: glioblastoma, anaplastic astrocytoma, colon cancer, prostate cancer, lung cancer, breast cancer, endometrial cancer, uterine cancer, ovarian cancer and said hematological cancer is leukemia.

21. The antibody, composition, or immunoconjugate for use according to any one of claims 17-20, wherein said treatment is of patients which are resistant or intolerant to previous treatment with at least one antitumor agent or wherein the treatment with an antitumor agent should be avoided.

22. A diagnostic method for measuring the amount of EPO protein in a sample previously obtained from a human or animal subject, comprising the step of using the antibody of any one of claims 1-6.

23. The diagnostic method according to claim 22, wherein said sample is chosen from the group consisting of cell, tissue, blood, saliva and cerebrospinal fluid.

24. The diagnostic method according to any one of claims 22 or 23, wherein said diagnostic method is carried out by one or more of: ELISA assay, Western blot analysis, RealTime PCR or PCR, functional angiogenesis assays and drug screening platform alone or in combination.

25. The diagnostic method according to any one of claims 22 to 24, comprising the further use of EPO receptors.

26. The diagnostic method according to claim 25, wherein said EPO receptors are chosen from the group consisting of EPOR, EPHB4, CSFR2B, tissue protection factor (TPR, EPOR/CD131 heterodimer).

27. A pharmaceutical kit comprising the antibody of any one of claims 1-6 and one or more compounds chosen from the group consisting of:
a negative functional modulator of S1 P signaling;
an anti-EPO receptor selected from the group comprising of EPOR, EPHB4, CSFR2B, tissue protection factor (TPR, EPOR/CD131 heterodimer); and/or
EPO mimetics,
for simultaneous, separate or sequential administration.

28. A hybridoma which is deposited under deposit Accession No. DSM ACC 3370 by the International Deposit Authority DSMZ.

29. An anti-Erythropoietin (EPO) monoclonal antibody produced by the hybridoma according to claim 28.

30. The anti-Erythropoietin (EPO) monoclonal antibody according to claim 29, wherein said antibody comprises:
a. a variable domain of a light chain (VL) having the amino acid sequence of SEQ ID NO:6;
and
b. a variable domain of a heavy chain (VH) having the amino acid sequence of SEQ ID NO:14.


SEQUENCE LISTING (17 sequences)

SEQ ID NO: 1 - DNA, 30 bp, Homo sapiens
  misc_feature 1..30: note = VL-CDR1
  source 1..30: mol_type = other DNA
gaaagtgttgactattatggcacaggttta

SEQ ID NO: 2 - DNA, 27 bp, Homo sapiens
  misc_feature 1..27: note = VL-CDR3
  source 1..27: mol_type = other DNA
cagcaaactaggaaggttccttcgacg

SEQ ID NO: 3 - DNA, 333 bp, Homo sapiens
  misc_feature 1..333: note = VL DNA sequence
  source 1..333: mol_type = other DNA
gatatcgttctcactcaatctccagcttctttggctgtgtctctagggcagagagccaccatctcctgcagagccagtgaaagtgttgactattatggcacaggtttaatgcagtggtaccaacagagaccaggacagccacccaaactcctcatctatggtgcatccaacgtaggatctggggtccctgccaggtttagcggcagtgggtctgggacagacttcagcctcaacatccatcctgtggagggggatgatattgcaatgtatttctgtcagcaaactaggaaggttccttcgacgttcggtggaggcaccaagttggaaatcaaa

SEQ ID NO: 4 - protein, 10 aa, Homo sapiens
  REGION 1..10: note = VL-CDR1
ESVDYYGTGL

SEQ ID NO: 5 - protein, 9 aa, Homo sapiens
  REGION 1..9: note = VL-CDR3
QQTRKVPST

SEQ ID NO: 6 - protein, 111 aa, Homo sapiens
  REGION 1..111: note = VL amino acid sequence
DIVLTQSPASLAVSLGQRATISCRASESVDYYGTGLMQWYQQRPGQPPKLLIYGASNVGSGVPARFSGSGSGTDFSLNIHPVEGDDIAMYFCQQTRKVPSTFGGGTKLEIK

SEQ ID NO: 7 - DNA, 24 bp, Homo sapiens
  misc_feature 1..24: note = VH-CDR1
  source 1..24: mol_type = other DNA
ggattcactttcagtacctatacc

SEQ ID NO: 8 - DNA, 24 bp, Homo sapiens
  misc_feature 1..24: note = VH-CDR2
  source 1..24: mol_type = other DNA
attagtaatggtggtgatagaacc

SEQ ID NO: 9 - DNA, 42 bp, Homo sapiens
  misc_feature 1..42: note = VH-CDR3
  source 1..42: mol_type = other DNA
gcaagacataatattactacggttccctttactatggactac

SEQ ID NO: 10 - DNA, 363 bp, Homo sapiens
  misc_feature 1..363: note = VH DNA Sequence
  source 1..363: mol_type = other DNA
gaggtgaagctgcaggagtctgggggaggtttagtgcagcctggagggtccctgaaactctcctgtgcagcctctggattcactttcagtacctataccatgtcttgggttcgccagactccagagaagaggctggagtgggtcgcatacattagtaatggtggtgatagaacctactatccagacactgtaaagggccgattcaccatctccagagacgatgccaagaacaccctgttcctgcaaatgagcagtctgaagtctgaggacacggccatgtattactgtgcaagacataatattactacggttccctttactatggactactggggtcaaggaacctcagtcaccgtctcctca

SEQ ID NO: 11 - protein, 8 aa, Homo sapiens
  REGION 1..8: note = VH-CDR1
GFTFSTYT

SEQ ID NO: 12 - protein, 8 aa, Homo sapiens
  REGION 1..8: note = VH-CDR2
ISNGGDRT

SEQ ID NO: 13 - protein, 14 aa, Homo sapiens
  REGION 1..14: note = VH-CDR3
ARHNITTVPFTMDY

SEQ ID NO: 14 - protein, 121 aa, Homo sapiens
  REGION 1..121: note = VH amino acid sequence
EVKLQESGGGLVQPGGSLKLSCAASGFTFSTYTMSWVRQTPEKRLEWVAYISNGGDRTYYPDTVKGRFTISRDDAKNTLFLQMSSLKSEDTAMYYCARHNITTVPFTMDYWGQGTSVTVSS

SEQ ID NO: 15 - protein, 193 aa, Homo sapiens
  REGION 1..193: note = EPO amino acid sequence (N-terminal signal peptide + proteinchain) aa 1-193
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 16 - protein, 166 aa, Homo sapiens
  REGION 1..166: note = EPO mature peptide amino acid sequence (aa 28-193)
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 17 - DNA, 2901 bp, Homo sapiens
  misc_feature 1..2901: note = EPO gene sequence
  source 1..2901: mol_type = other DNA
cccggagccggaccggggccaccgcgcccgctctgctccgacaccgcgccccctggacagccgccctctcctccaggcccgtggggctggccctgcaccgccgagcttcccgggatgagggcccccggtgtggtcacccggcgcgccccaggtcgctgagggaccccggccaggcgcggagatgggggtgcacggtgagtactcgcgggctgggcgctcccgcccgcccgggtccctgtttgagcggggatttagcgccccggctattggccaggaggtggctgggttcaaggaccggcgacttgtcaaggaccccggaagggggaggggggtggggcagcctccacgtgccagcggggacttgggggagtccttggggatggcaaaaacctgacctgtgaaggggacacagtttgggggttgaggggaagaaggtttgggggttctgctgtgccagtggagaggaagctgataagctgataacctgggcgctggagccaccacttatctgccagaggggaagcctctgtcacaccaggattgaagtttggccggagaagtggatgctggtagctgggggtggggtgtgcacacggcagcaggattgaatgaaggccagggaggcagcacctgagtgcttgcatggttggggacaggaaggacgagctggggcagagacgtggggatgaaggaagctgtccttccacagccacccttctccctccccgcctgactctcagcctggctatctgttctagaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtgagaccccttccccagcacattccacagaactcacgctcagggcttcagggaactcctcccagatccaggaacctggcacttggtttggggtggagttgggaagctagacactgcccccctacataagaataagtctggtggccccaaaccatacctggaaactaggcaaggagcaaagccagcagatcctacggcctgtgggccagggccagagccttcagggacccttgactccccgggctgtgtgcatttcagacgggctgtgctgaacactgcagcttgaatgagaatatcactgtcccagacaccaaagttaatttctatgcctggaagaggatggaggtgagttcctttttttttttttttcctttcttttggagaatctcatttgcgagcctgattttggatgaaagggagaatgatcgagggaaaggtaaaatggagcagcagagatgaggctgcctgggcgcagaggctcacgtctataatcccaggctgagatggccgagatgggagaattgcttgagccctggagtttcagaccaacctaggcagcatagtgagatcccccatctctacaaacatttaaaaaaattagtcaggtgaggtggtgcatggtggtagtcccagatatttggaaggctgaggcgggaggatcgcttgagcccaggaatttgaggctgcagtgagctgtgatcacaccactgcactccagcctcagtgacagagtgaggccctgtctcaaaaaagaaaagaaaaaagaaaaataatgagggctgtatggaatacattcattattcattcactcactcactcactcactcattcattcattcattcattcaacaagtcttattgcataccttctgtttgctcagcttggtgcttggggctgctgaggggcaggagggagagggtgacatgggtcagctgactcccagagtccactccctgtaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgtaagaaggggagaagggtcttgctaaggagtacaggaactgtccgtattccttccctttctgtggcactgcagcgacctcctgttttctccttggcagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatgaccaggtgtgtccacctgggcatatccaccacctccctcaccaacattgcttgtgccacaccctcccccgccactcctgaaccccgtcgaggggctctcagctcagcgccagcctgtcccatggacactccagtgccagcaatgacatctcaggggccagaggaactgtccagagagcaactctgagatctaaggatgtcacagggccaacttgagggcccagagcaggaagcattcagagagcagctttaaactcagggacagagccatgctgggaagacgcctgagctcactcggcaccctgcaaaatttgatgccaggacacgctttggaggcgatttacctgttttcgcacctaccatcagggacaggatgacctggataacttaggtggcaagctgtgacttctccaggtctcacgggcatgggcactcccttggtggcaagagcccccttgacaccggggtggtgggaaccatgaagacaggatgggggctggcctctggctctcatggggtccaagttttgtgtattcttcaacctcattgacaagaactgaaaccaccaa